(19) United States
(12) Patent Application Publication
(10) Pub. No.: US 2008/0070247 A1
(43) Pub. Date: Mar. 20, 2008

(54) PHYSIOGENOMIC METHOD FOR PREDICTING EFFECTS OF EXERCISE

(76) Inventors: Gualberto RUANO, Milford, CT (US); Andreas WINDEMUTH, Woodbridge, CT (US); Paul D. THOMPSON, Simsbury, CT (US)

Correspondence Address: MORGAN & FINNEGAN, L.L.P., 3 WORLD FINANCIAL CENTER, NEW YORK, NY, 10281-2101, US

(21) Appl. No.: 11/532,418

(22) Filed: Sep. 15, 2006

(51) Int. Cl. C12Q 1/68; C07H 21/04

(52) U.S. Cl. 435/6; 536/24.3

(57) ABSTRACT

The present invention relates to the use of genetic variants of associated marker genes to predict an individual's response to exercise. The present invention further relates to analytical assays and computational methods using the novel marker gene set. The present invention has utility for developing personalized fitness regimens to optimize physiological response.

### FIELD OF THE INVENTION

The invention is in the field of physiological genomics, hereafter referred to as “physiogenomics.” More specifically, the invention relates to the use of genetic variants of associated genes to predict the effects of an exercise treatment regimen on lipid metabolism and serum lipids and lipoproteins in patients.

### BACKGROUND OF THE INVENTION

The population-wide rise in obesity and obesity's ability to increase the risk of cardiovascular disease (CVD) threaten an alarming new epidemic. Because of this threat, physical fitness is now a major public health imperative. The beneficial effects of exercise for overall health and disease prevention are increasingly recognized. Yet, exercise regimens have been underutilized as a therapeutic strategy to prevent CVD. Several factors may contribute to this shortcoming. Exercise is frequently perceived as onerous by many patients, reducing compliance and its prescription by health professionals. Exercise may fail to mitigate a patient's risk factor, prompting the physician to use pharmacologic therapy and reducing the health professional's use of exercise as a treatment strategy in future patients. Conversely, accurate prediction of which patients will or will not benefit from either an exercise or pharmacologic intervention may minimize the reliance on costly, multiple drug regimens. Increased exercise compliance and adherence could arise from the patient's motivation to avoid drugs and their side effects thus increasing confidence in exercise treatments by physicians.

So far, pharmacological interventions to alter lipid and inflammatory profiles are presumed to be most powerful. Statins are widely prescribed to lower low-density lipoprotein (LDL) levels, fibrates to lower triglycerides (TGs), niacin to increase high density lipoprotein (HDL), and aspirin to decrease inflammation. All have side effects: statins, myalgias, muscle weakness, and rare life threatening rhabdomyolysis; niacin, flushing and hepatitis; fibrates, gall stones and increase in LDL; and aspirin, gastrointestinal complaints and bleeding. Simultaneously improving multiple risk factors such as LDL, TG, HDL, and C-reactive protein (CRP) and other lipid and inflammatory markers generally requires drug combinations, which produce more side effects than monotherapy.

In contrast, exercise, if therapeutically targeted and performed, achieves multi-system benefits including improved lipid and inflammatory profiles with few side effects. The best medical care will require multiple pharmacological and non-pharmacological strategies to treat and reduce CVD risk produced by complicated endocrine, lipid and inflammatory disorders including diabetes and the metabolic syndrome. Medications offer chemoprevention and pharmacological tools whereas physical activity could serve as a more primary “physiological” prevention and treatment approach.

However, changes in serum lipids with exercise training are often small and individually variable, limiting the role of exercise in treating lipid abnormalities. A meta-analysis of 59 exercise training studies reported an average increase in HDL-C of only 2 mg/dL (Tran Z V et al, JAMA 254:919 (1985)). Furthermore, exercise is less effective in increasing HDL and altering TG metabolism in individuals with initially elevated TGs and low HDL. Such observations suggest that individual differences contribute to the variability in the exercise response. It would therefore be desireable to provide a method for predicting whether exercise would have a beneficial effect on serum lipids and the clinical consequences thereof.

The field of physiogenomics offers an important approach for integrating genotype, phenotype, and population analysis of functional variability among individuals. In physiogenomics, genetic markers (e.g. single nucleotide polymorphisms or “SNPs”) are analyzed to discover statistical associations to physiological characteristics or outcomes in populations of individuals. It is therefore an object of the invention to provide physiogenomic markers for predicting physiological response to exercise by using an informatics platform to analyze data from exercise studies. It is a further object of the invention to provide an ensemble of SNP markers predictive of a variety of physiological responses to exercise to enable the identification of individuals that would respond most favorably to exercise on the basis of one or more physiological parameters.

### SUMMARY OF THE INVENTION

In accordance with the foregoing objectives and others, the principles of physiogenomics have been used to provide an ensemble of marker genes useful for predicting physiological response.

In one aspect of the invention, an ensemble of marker genes useful for predicting physiological response to exercise is provided. The ensemble comprising at least two single nucleotide polymorph (SNP) gene variants selected from the group consisting of: rs1041163; rs1042718; rs10460960; rs10508244; rs10513055; rs10515070; rs107540; rs10890819; rs131010; rs1143634; rs11503016; rs1171276; rs1255; rs1290443; rs1322783; rs1356413; rs1396862; rs1398176; rs1440451; rs167771; rs1799978; rs1800471; rs1800871; rs1801105; rs1801278; rs1801714; rs1805002; rs1891311; rs205590; rs2067477; rs2070424; rs2070586; rs2076672; rs2162189; rs2229126; rs2240403; rs2269935; rs2276307; rs2278718; rs2296189; rs2298122; rs2514869; rs2515449; rs322695; rs324651; rs334555; rs3756007; rs3760396; rs3822222; rs3917550; rs4121817; rs4149056; rs4520; rs4531; rs4675096; rs4726107; rs4792887; rs4917348; rs4933200; rs5049; rs5092; rs5361; rs563895; rs5896; rs600728; rs6078; rs6092; rs6131; rs659734; rs6700734; rs6967107; rs706713; rs707922; rs7200210; rs722341; rs7412; rs7556371; rs8178990; rs870995; rs885834; rs908867; and rs936960.

In another aspect of the invention, an ensemble of marker genes is provided, comprising:

at least two single nucleotide polymorphism (SNP) gene variants, the presence of which in a human correlates with at least one physiological response to exercise; wherein the physiological response is selected from the group consisting of log of blood triglyceride level; blood LDL cholesterol level; blood HDL cholesterol level; ratio of total cholesterol to HDL cholesterol; LDL cholesterol, small fraction level; HDL cholesterol, large fraction level; blood glucose level; systolic blood pressure; diastolic blood pressure; body mass; body mass index; fat percentage; weight normalized maximum oxygen uptake; maximum oxygen uptake; and combinations thereof; and wherein the at least two SNP gene variants comprise at least one SNP gene variant having a positive coefficient and at least one SNP gene variant having a negative coefficient in the phyiotype model, including:

(1) in the case where said physiological response is a change in blood LDL cholesterol level, the marker set comprises: (i) at least one SNP gene variant selected from the group consisting of rs334555, rs1799978, rs870995, rs1398176, and rs5092; and (ii) at least one SNP gene variant selected from the group consisting of rs3118536, rs2005590, rs1041163, rs1800471, and rs707922; and

(2) in the case where the physiological response is a change in blood HDL cholesterol level, the marker set comprises: (i) at least one SNP gene variant selected from the group consisting of rs660339, rs894251, rs3760396, rs10513055, rs10513055, rs1800871, rs3760396, and rs1891311; and (ii) at least one SNP gene variant selected from the group consisting of rs936960, rs1143634, rs5049, and rs1891311; and

(3) in the case where the physiological response is a change in log of blood triglyceride level, the marker set comprises: (i) at least one SNP gene variant selected from the group consisting of rs722341, rs7602, rs4121817, rs5880, rs908867, rs2278718, rs2240403, and rs1171276; and (ii) at least one SNP gene variant selected from the group consisting of rs563895, rs2070586, rs1800871, rs2070586, rs10460960, rs2276307, rs11503016, and rs563895; and

(4) in the case where the physiological response is a change in blood glucose level, the marker set comprises: (i) at least one SNP gene variant selected from the group consisting of rs737865, rs10082776, rs10508244, rs1322783, rs2070424, rs107540, rs1042718, rs5361, and rs322695; and (ii) at least one SNP gene variant selected from the group consisting of rs1398176, rs722341, rs3822222, and rs2229126; and

(5) in the case where the physiological response is a change in LDL cholesterol, small fraction level, the marker set comprises: (i) at least one SNP gene variant selected from the group consisting of rs2033447, rs1877394, rs4917348, rs1131010, rs706713, rs4675096, and rs4917348; and (ii) at least one SNP gene variant selected from the group consisting of rs1045642, rs6131, rs2076672, rs6092, rs6078, rs659734, and rs885834; and

(6) in the case where the physiological response is a change in HDL cholesterol, large fraction level, the marker set comprises: (i) at least one SNP gene variant selected from the group consisting of rs10513055, rs1800871, and rs3760396; and (ii) at least one SNP gene variant selected from the group consisting of rs1799978, rs8192708, rs521674, rs5049, rs1042718, and rs4520; and

(7) in the case where the physiological response is a change in systolic blood pressure, the marker set comprises: (i) at least one SNP gene variant selected from the group consisting of rs597316, rs10515070, rs4149056, rs2298122, and rs6967107; and (ii) at least one SNP gene variant selected from the group consisting of rs2070424, rs6586179, rs1064344, rs11100494, rs1800871, rs1801105, rs7200210, and rs4726107; and

(8) in the case where the physiological response is a change in diastolic blood pressure, the marker set comprises: (i) at least one SNP gene variant selected from the group consisting of rs722341, rs3762272, rs600728, rs7556371, rs4531, and rs2067477; and (ii) at least one SNP gene variant selected from the group consisting of rs660339, rs662, rs2162189, rs2702285, and rs324651.

(9) in the case where the physiological response is a change in body mass, the marker set comprises: (i) at least one SNP gene variant selected from the group consisting of rs870995, rs600728, rs676643, rs2070424, rs1801278, rs6700734, and rs4792887; and (ii) at least one SNP gene variant selected from the group consisting of rs6541017, rs1041163, rs722341, rs2162189, rs1255, rs1440451, and rs3756007; and

(10) in the case where the physiological response is a change in body mass index, the marker set comprises: (i) at least one SNP gene variant selected from the group consisting of rs5880, rs600728, rs676643, rs2070424, rs1801278, and rs4792887; and (ii) at least one SNP gene variant selected from the group consisting of rs132642, rs2162189, rs1440451, rs936960, and rs167771; and

(11) in the case where the physiological response is a change in percentage fat, the marker set comprises: (i) at least one SNP gene variant selected from the group consisting of rs676643, rs2070424, rs885834, rs8178990, and rs600728; and (ii) at least one SNP gene variant selected from the group consisting of rs8192708, rs6312, rs722341, and rs1290443; and

(12) in the case where the physiological response is a change in weight normalized maximum oxygen uptake, the marker set comprises: (i) at least one SNP gene variant selected from the group consisting of rs8178990, rs5447, rs1800871, rs4149056, rs7412, and rs1901714; and (ii) at least one SNP gene variant selected from the group consisting of rs2298122, rs26312, rs563895, rs5896, rs3917550, rs2296189, and rs1356413; and

(13) in the case where the physiological response is a change in maximum oxygen uptake, the marker set comprises: (i) at least one SNP gene variant selected from the group consisting of rs11503016, rs2515449, rs334555, rs722341, rs4149056, rs7412, rs1396862, rs2515449, and rs1805002; and (ii) at least one SNP gene variant selected from the group consisting of rs597316, rs26312, rs2020933, rs563895, and rs5896.

In yet another aspect of the invention arrays including any or all of the foregoing markers are also provided. The arrays may be provided on a solid support or the like.

In a further aspect of the invention, a method of predicting an individual's physiological response to exercise is also provided comprising (1) obtaining genetic material from the individual; and (2) assaying the genetic material for the presence of the at least two SNP gene variants of the foregoing ensemble.

These and other aspects of the present invention will be better understood upon a reading of the following detailed description when considered in connection with the accompanying figures.

### BRIEF DESCRIPTION OF THE DRAWINGS

FIG. 1. Distribution of the baseline physiological responses and percent change with the reference range indicated for the following responses: baseline LDL and % change in LDL; baseline HDL and % change in HDL; baseline triglycerides as log(tg) and % change in log(tg); baseline blood glucose (glu) and % change in blood glucose; baseline LDL, small fraction (ldlsm) and % change in LDL, small fraction; baseline HDL, large fraction (hdllg) and % change in HDL, large fraction; baseline systolic blood pressure (sbp) and % change in systolic blood pressure; baseline diastolic blood pressure (dbp) and % change in diastolic blood pressure; baseline body mass (bms) and % change in body mass; baseline body mass index (bmi) and % change in body mass index; baseline waist size and % change in waist size; baseline percent fat (pcfat) and % change in percent fat; baseline percent fat (pcfat) and % change in percent fat; baseline weight normalized maximum oxygen uptake (vmax) and % change in weight normalized maximum oxygen uptake; and baseline maximum oxygen uptake (vmaxl) and % change in maximum oxygen uptake.

FIG. 2. Individual genotypes (circles) of the indicated SNPs overlaid on the distribution of change in physiological response (thin line) for the physiological responses of change in LDL; change in HDL; change in log(tg); change in blood glucose (glu); change in LDL, small fraction (ldlsm); change in HDL, large fraction (hdllg); change in systolic blood pressure (sbp); change in diastolic blood pressure (dbp); change in body mass (bms); change in body mass index (bmi); change in waist size (waist); change in percent fat (pcfat); change in weight normalized maximum oxygen uptake (vmax); and change in maximum oxygen uptake (vmaxl). Each circle represents a subject, with the horizontal axis specifying the change in physiological response, and the vertical axis the genotype: bottom—homozygous for major allele, middle—heterozygous, top—homozygous for minor allele. A LOESS (LOcally wEighted Scatter plot Smooth) fit of the allele frequency as a function of change in body mass (thick line) is shown.

FIG. 3 shows the response distribution corresponding to change in body mass (bms) as the result of exercise for the individuals in a reference population whose genetic data was used to form a physiogenomic database. More specifically, FIG. 3 shows a 40 SNP ensemble (represented as one per row) for 40 individuals (represented as one per column) in a reference population. Each square is a genotype for a person for one of the SNPs in the ensemble. The color coding is as follows: Black-homozygous, Gray-heterozygous genotypes. The 20 individuals to the left of the figure are representative of the bottom quartile of response rankings. The 20 individuals on the right of the figure are representative of the upper quartile of response rankings.

FIG. 4 shows a representational display of an individual patient's predicted response to exercise.

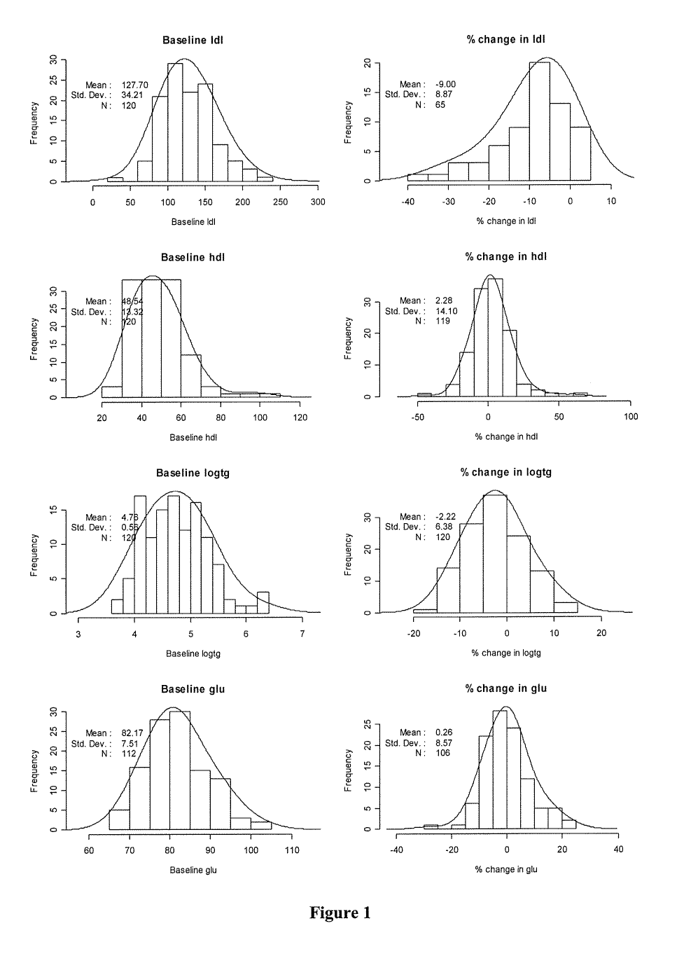

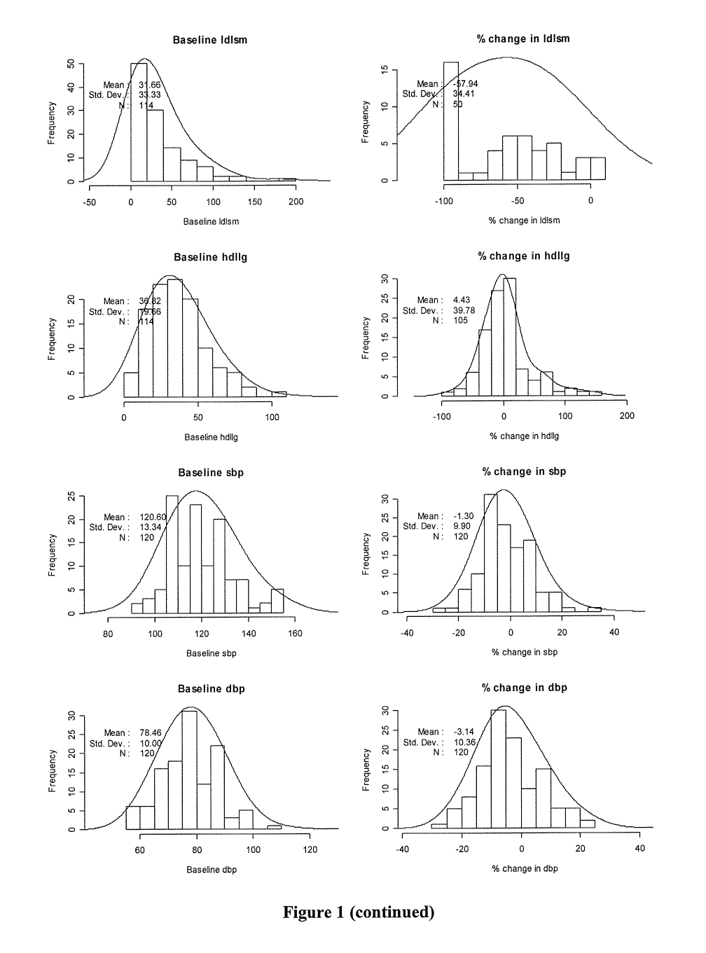

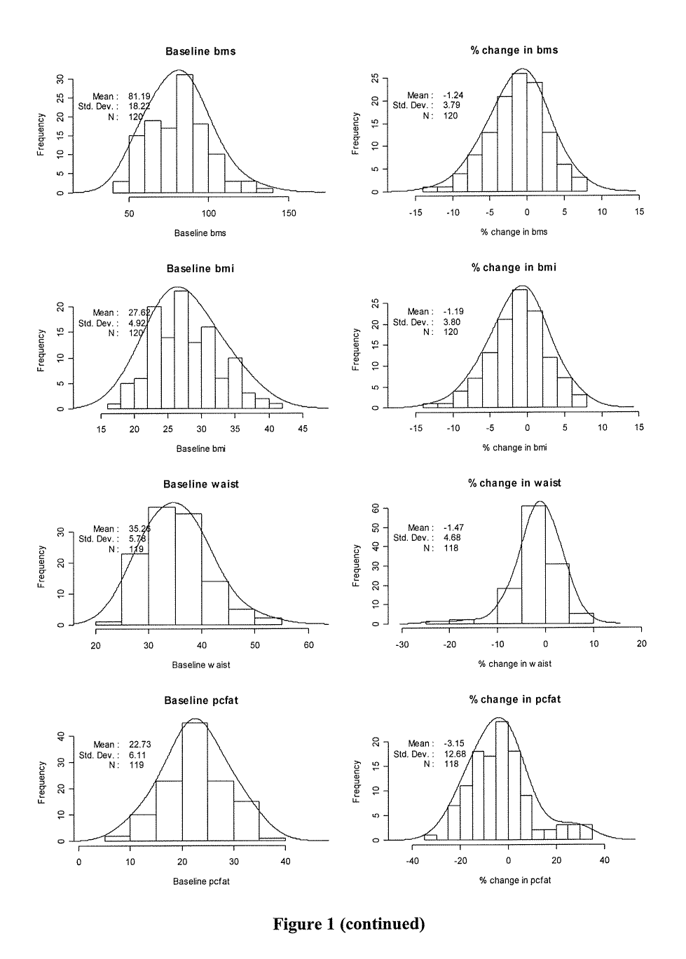

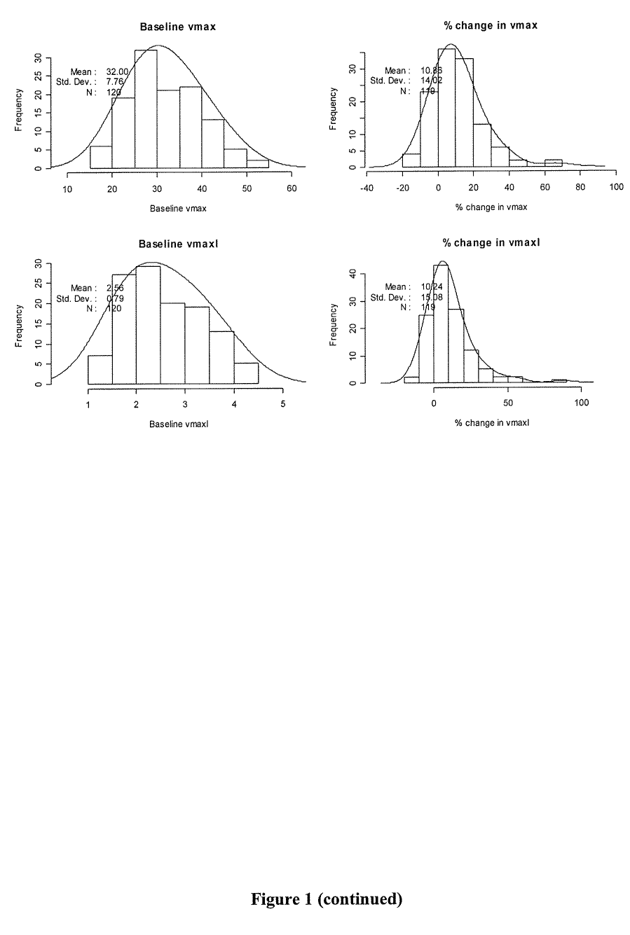

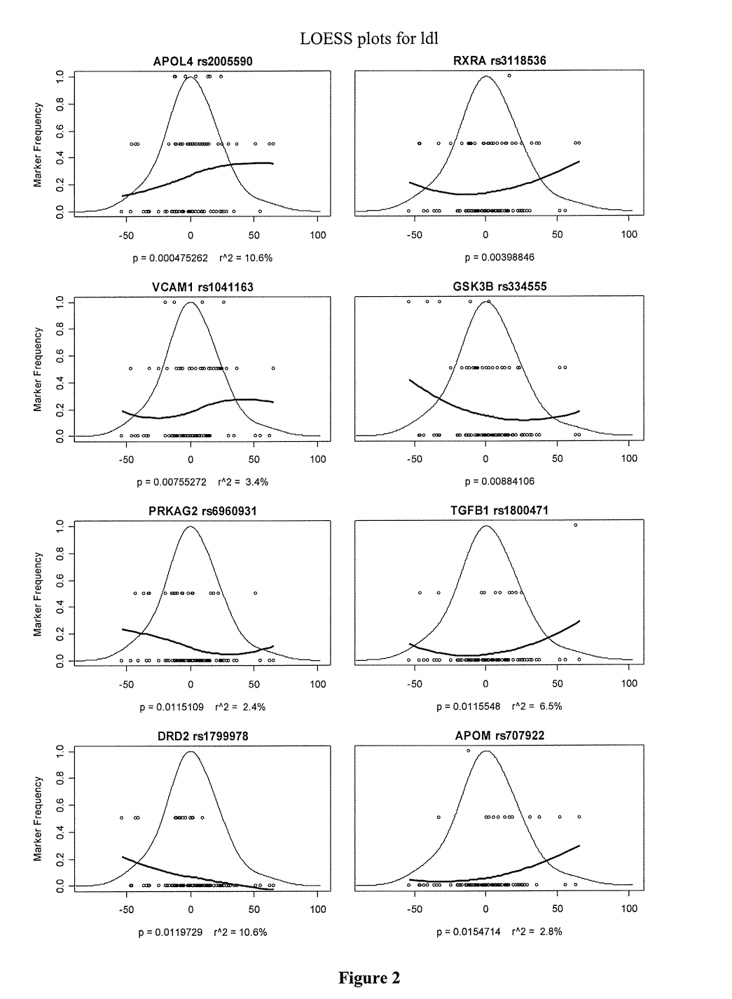

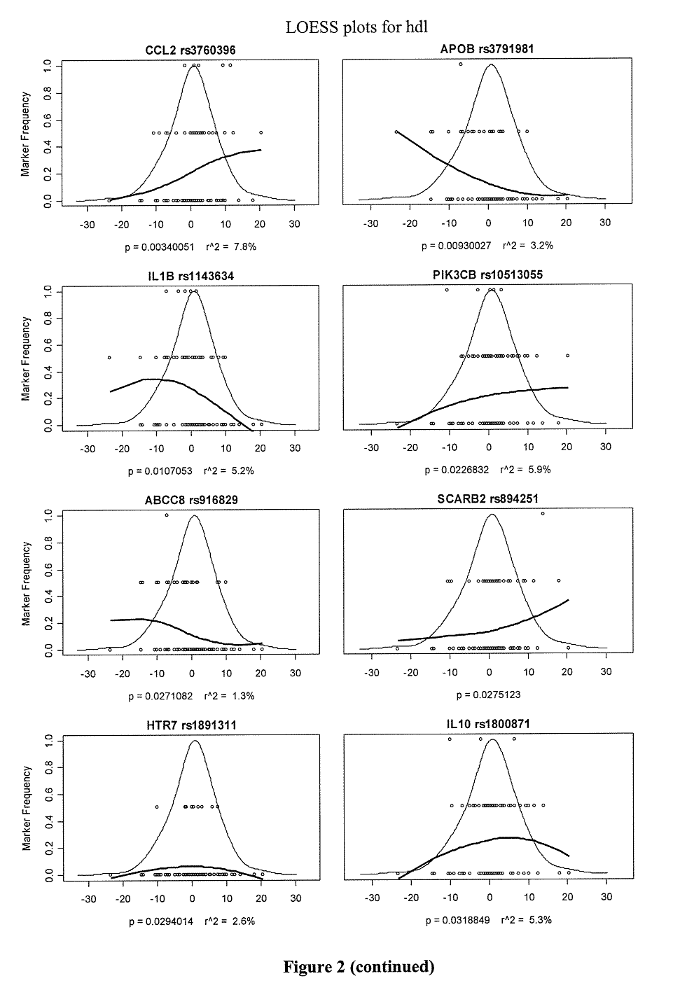

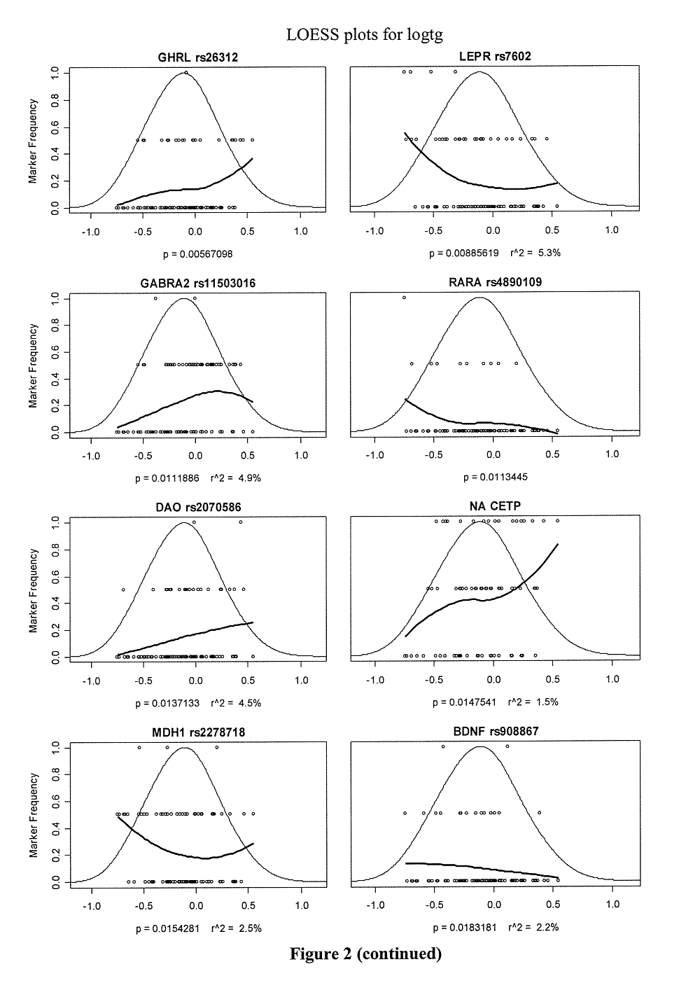

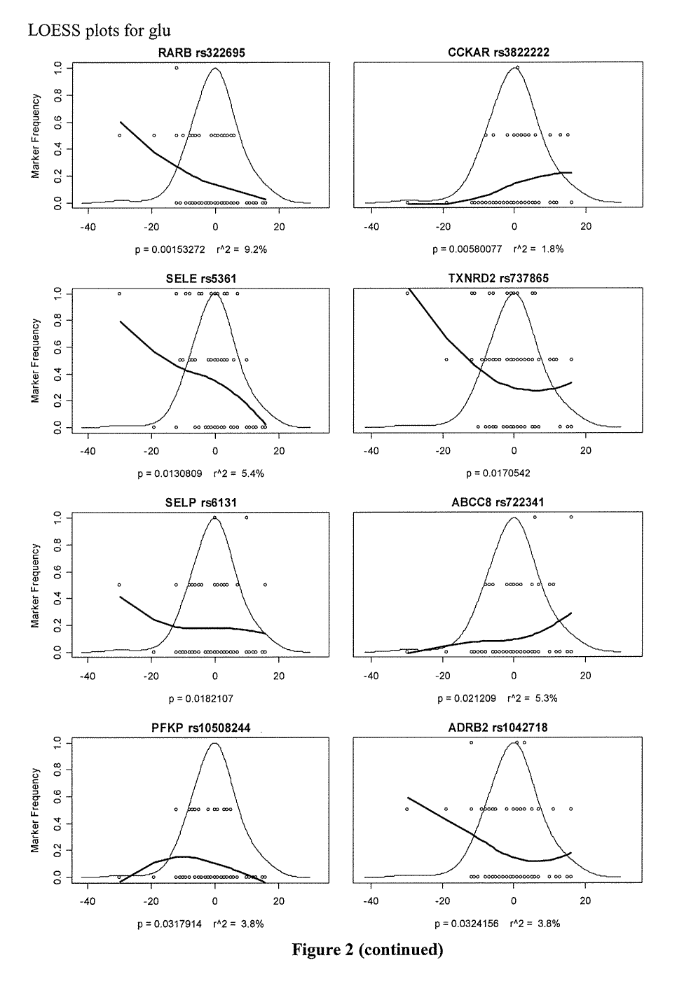

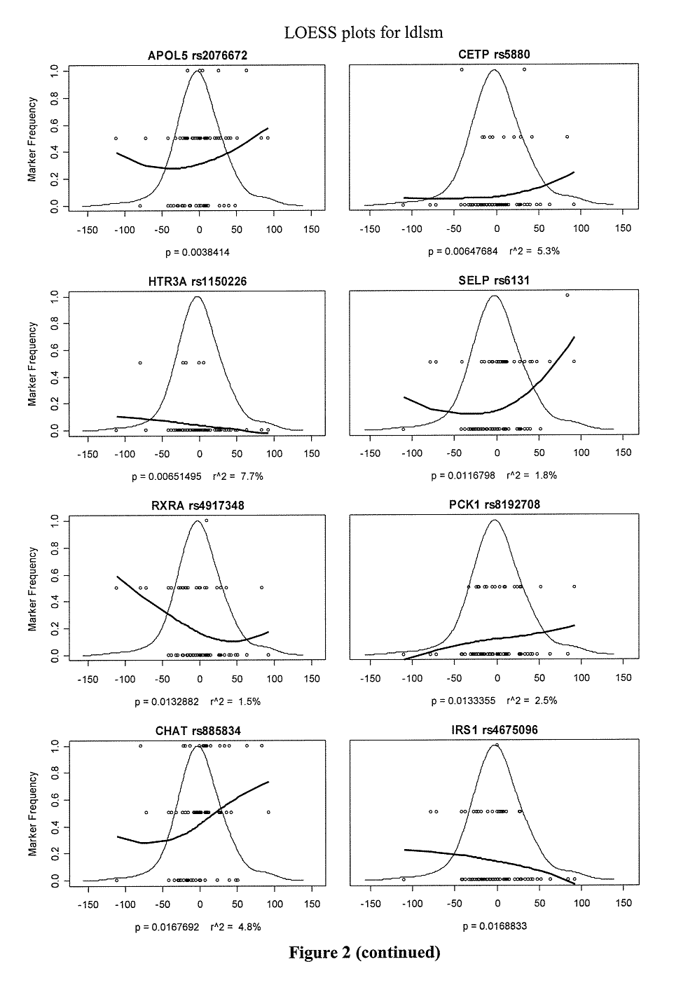

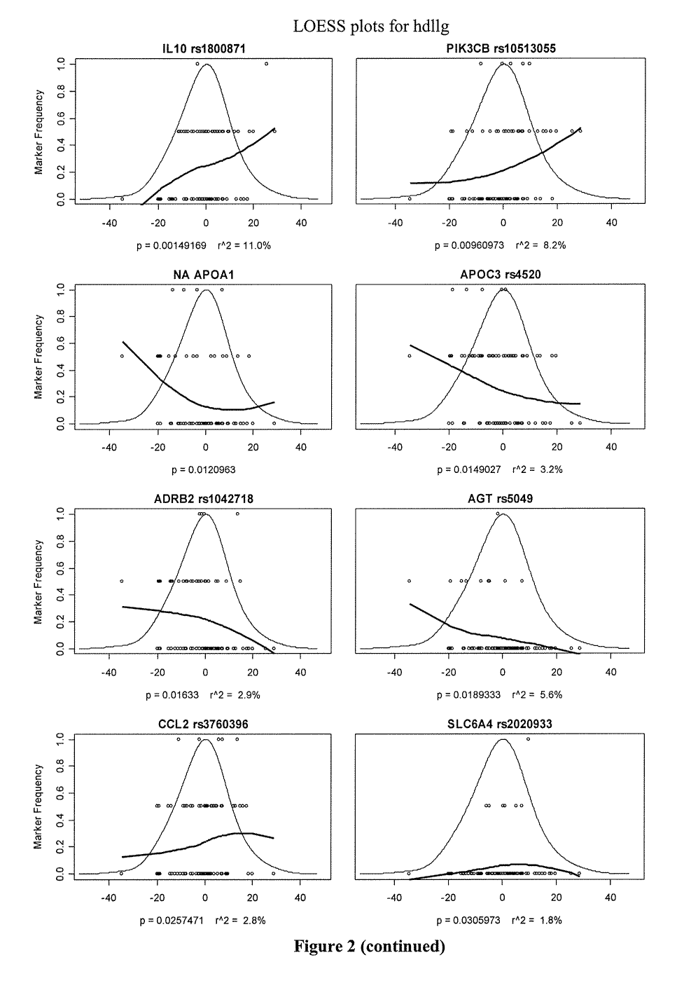

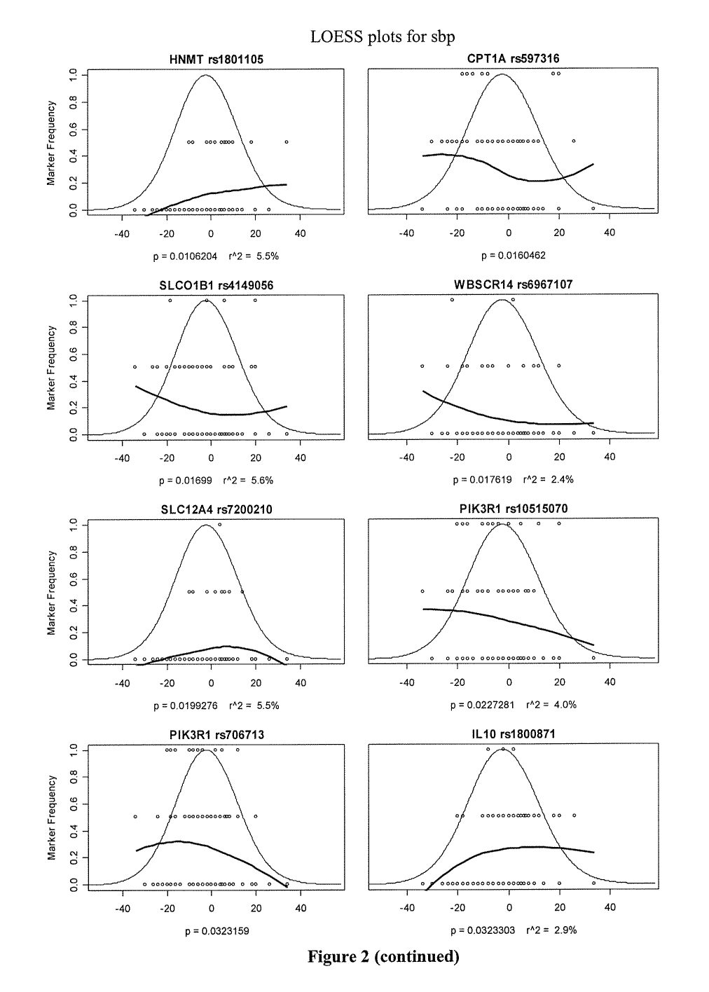

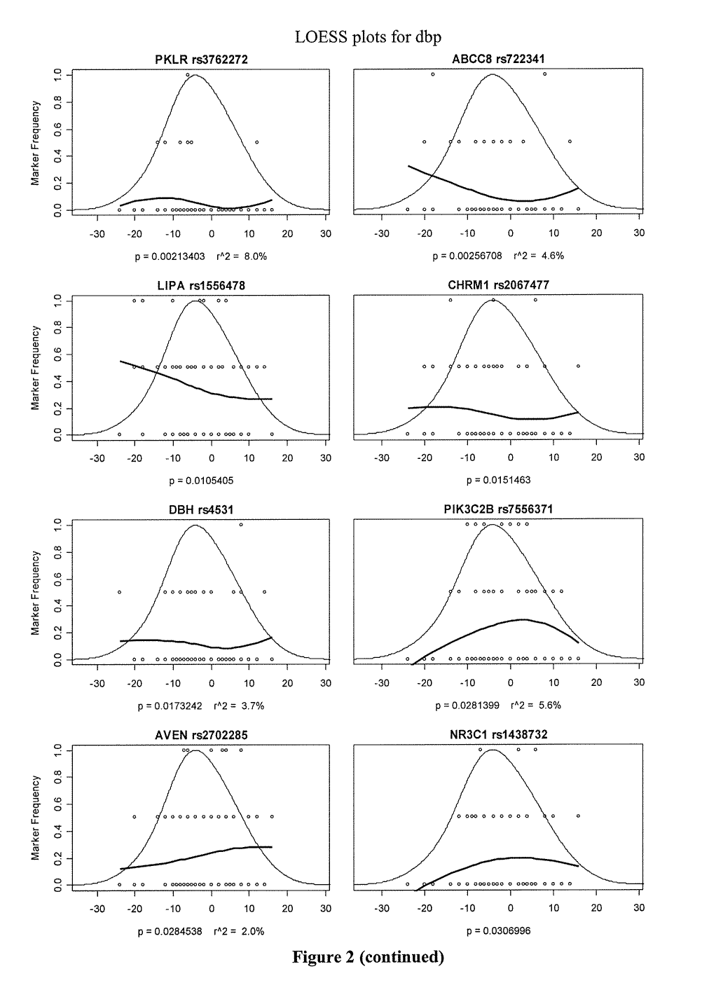

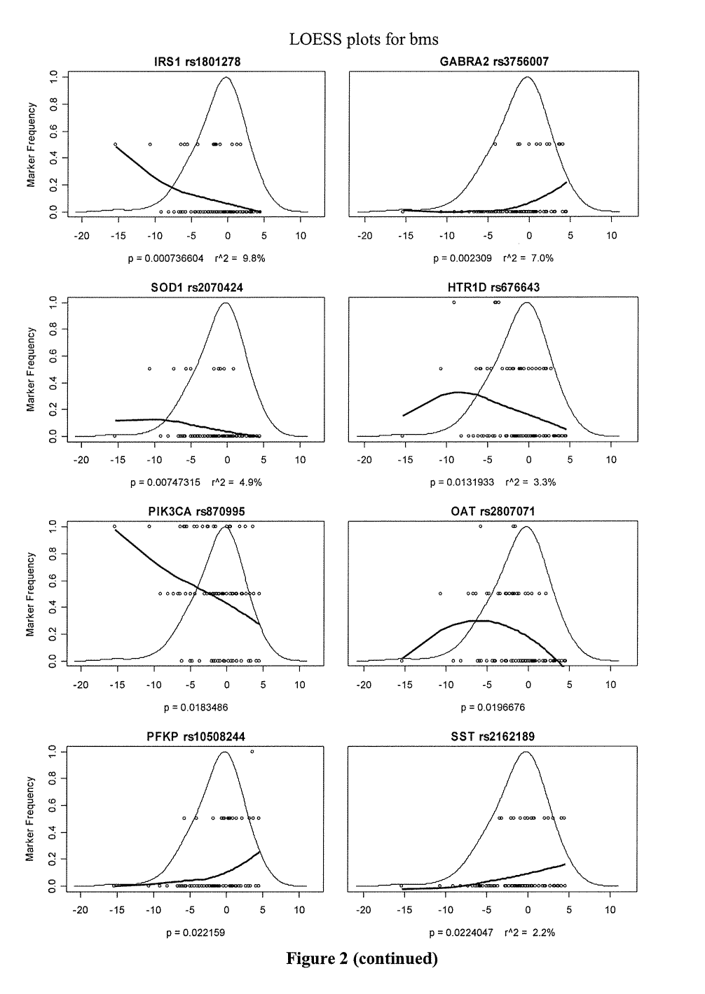

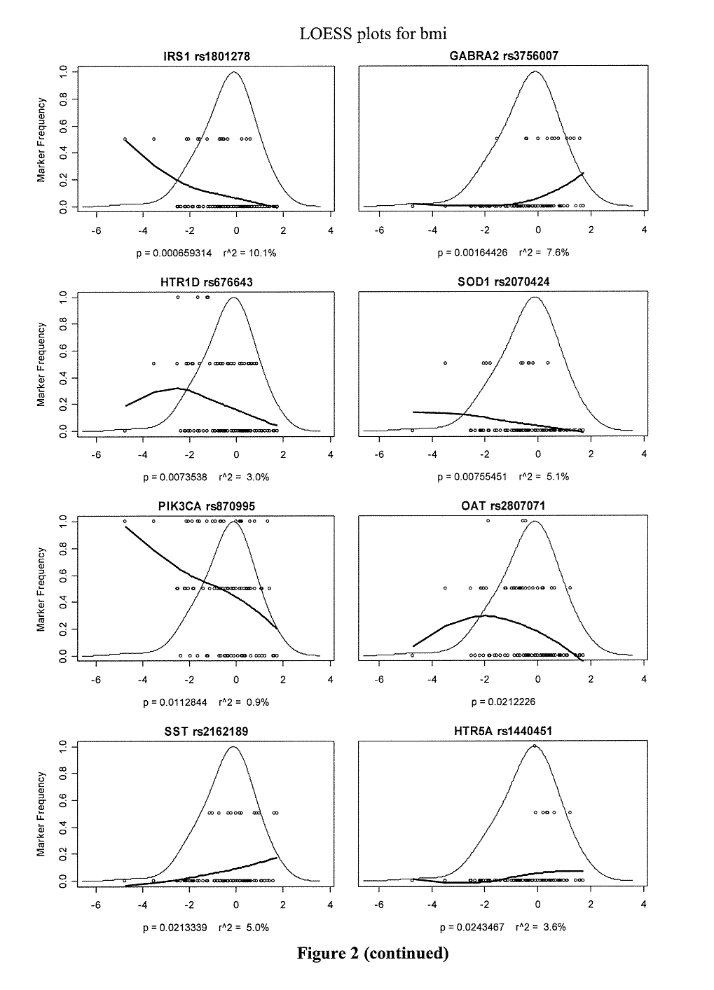

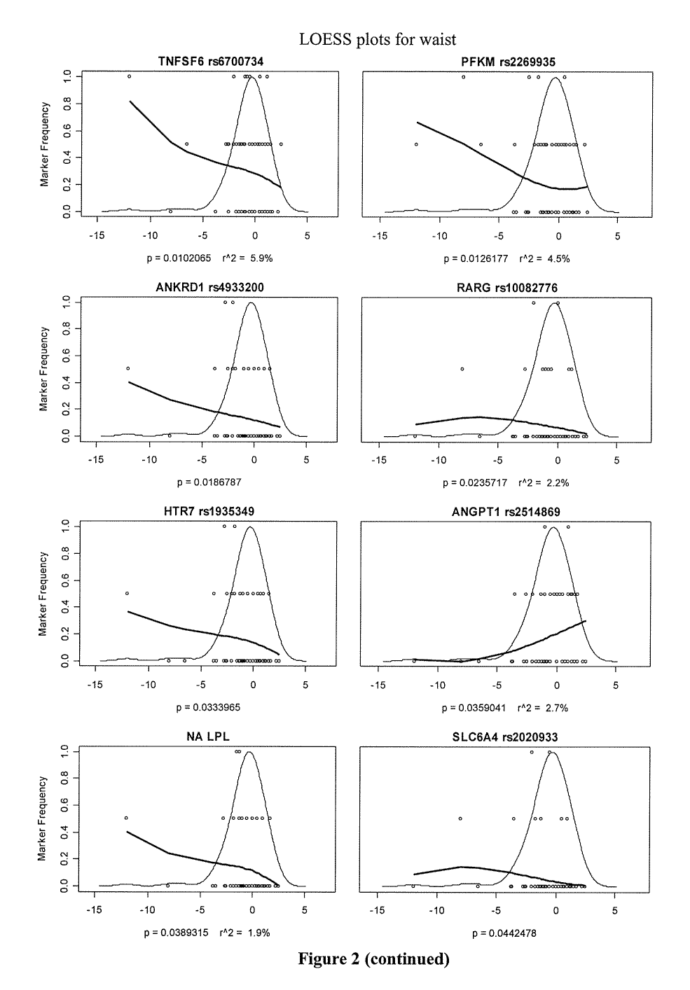

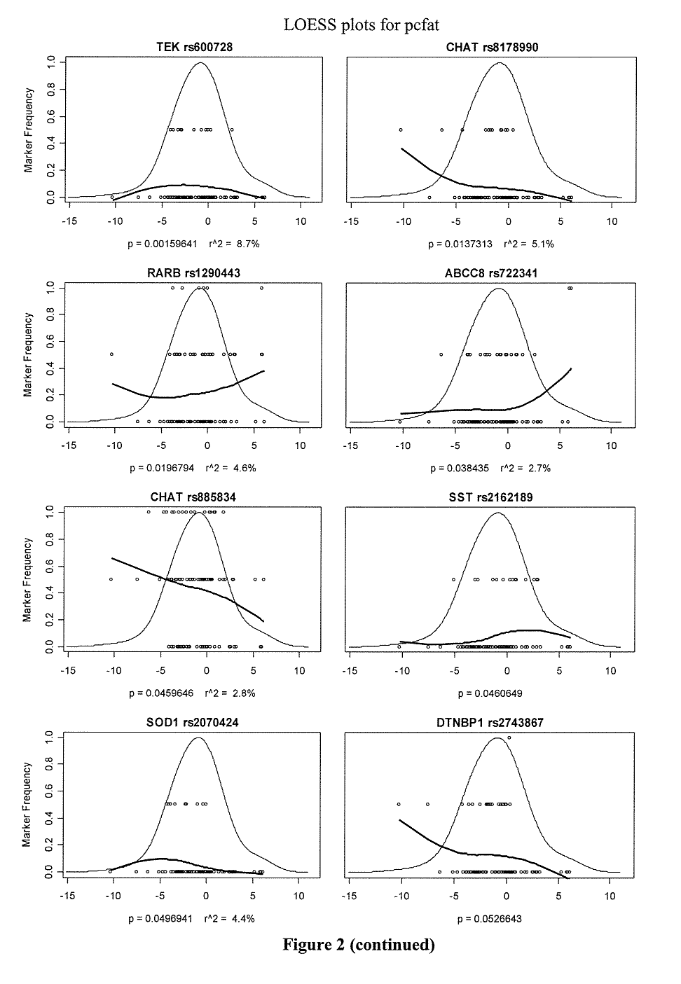

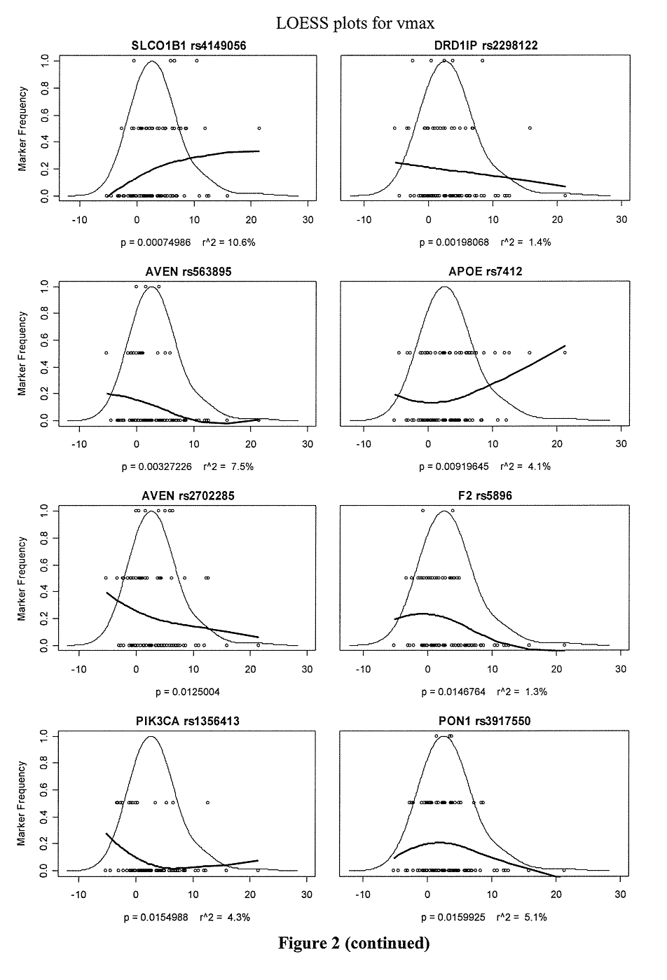

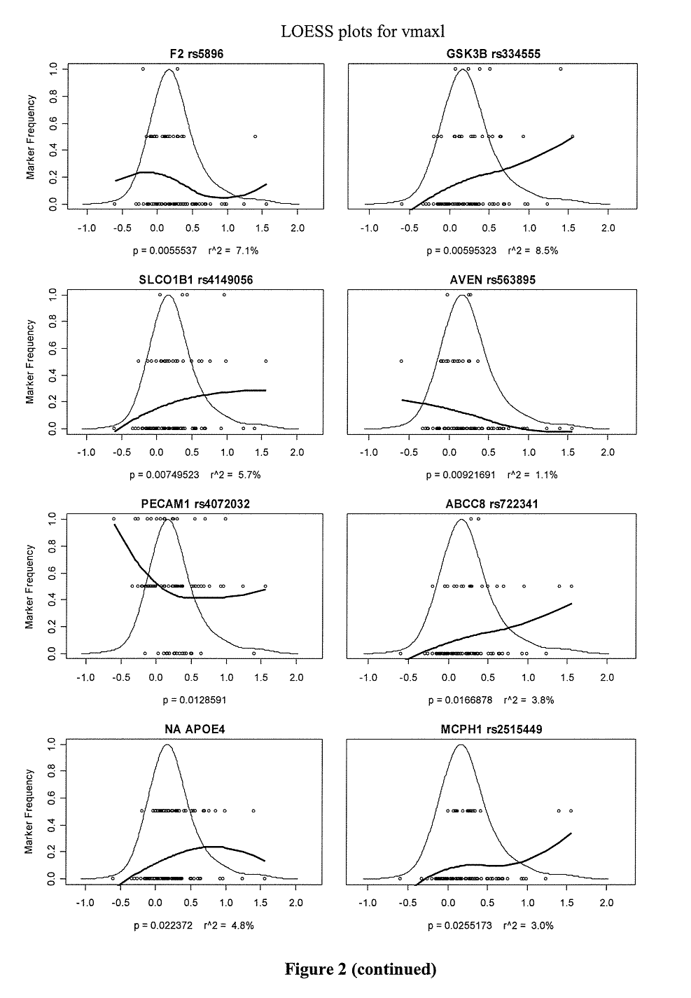

### DETAILED DESCRIPTION

We have invented a genotype-based method for predicting positive effects of exercise training on a clinical outcome, with the desired clinical outcome including, for example, increase in HDL-C at the expense of LDL-C in subjects. The predictive method is based on allelic variants of a set of marker biochemicals and is applicable to all humans, not only those with CVD (Thompson, P D et al., Metabolism 53:193 (2/2004)).

The following definitions will be used in the specification and claims:
1. Correlations or other statistical measures of relatedness between DNA marker ensembles and physiologic parameters are as used by one of ordinary skill in this art.
2 As use herein, “polymorphism” refers to DNA sequence variations in the cellular genomes of animals, preferably mammals. Such variations include mutations, single nucleotide changes, insertions and deletions. Single nucleotide polymorphism (“SNP”) refers to those differences among samples of DNA in which a single nucleotide pair has been substituted by another.
3. As used herein, “variants” or “variance” is synonymous with polymorphism.
4. As used herein, “phenotype” refers to any observable or otherwise measurable physiological, morphological, biological, biochemical or clinical characteristic of an organism. The point of genetic studies is to detect consistent relationships between phenotypes and DNA sequence variation (genotypes).
5. As used herein, “genotype” refers to the genetic composition of an organism. More specifically, “genotyping” as used herein refers to the analysis of DNA in a sample obtained from a subject to determine the DNA sequence in one or more specific regions of the genome, for example, at a gene that influences a disease or drug response.
6. As used herein, the term “associated with” in connection with a relationship between a genetic characteristic (e.g., a gene, allele, haplotype or polymorphism) and a disease or condition means that there is a statistically significant level or relatedness based on any accepted statistical measure of relatedness.
7. As used herein, a “gene” is a sequence of DNA present in a cell that directs the expression of biochemicals, i.e., proteins, through, most commonly, a complimentary RNA.

It has surprisingly been found that physiogenomic methods can be employed to identify genetic markers associated with physiological response to exercise. Thus, a patient can be assayed for the presence of one or more of genetic markers and a personalized predicted response profile developed based on the presence or absence of the marker, the specific allele (i.e., heterozygous or homozygous), and the predictive ability of the marker.

The physiogenomics methods employed in the present invention are described generally in U.S. patent application Ser. No. 11/371,511 and U.S. patent application Ser. No. 11/010,716, both of which are hereby incorporated by reference. Briefly, the physiogenomics method for predicting whether a particular exercise regimen will produce a beneficial effect on a patient typically comprises (a) selecting a plurality of genetic markers based on an analysis of the entire human genome or a fraction thereof; (b) identifying significant covariates among demographic data and the other phenotypes preferably by linear regression methods (e.g., R² analysis or principal component analysis); (c) performing for each selected genetic marker an unadjusted association test using genetic data; (d) optionally using permutation testing to obtain a non-parametric and marker complexity independent probability (“p”) value for identifying significant markers, wherein p denotes the probability of a false positive, and the significance is shown by p<0.10, more preferably p<0.05, and even more preferably p<0.01, and even more preferably p<0.001; (e) constructing a physiogenomic model by multivariate linear regression analyses and model parameterization for the dependence of the patient's response to exercise with respect to the markers, wherein the physiogenomic model has p<0.10, preferably p<0.05, and more preferably p<0.01, and even more preferably p<0.001; and (f) identifying one or more genes not associated with a particular outcome in the patient to serve as a physiogenomic control.

The physiogenomic method was used to identify an ensemble of markers which is predictive of a variety of physiological responses to exercise, including log of blood triglyceride level; blood LDL cholesterol level; blood HDL cholesterol level; ratio of total cholesterol to HDL cholesterol; LDL cholesterol, small fraction level; HDL cholesterol, large fraction level; blood glucose level; systolic blood pressure; diastolic blood pressure; body mass; body mass index; fat percentage; weight normalized maximum oxygen uptake; and maximum oxygen uptake.

The ensemble of marker genes will comprise one or more, preferably, two or more, and more preferred still, a plurality of gene variants. Preferred variants in accordance with the invention are single nucleotide polymorphisms (SNPs) which refers to a gene variant differing in the identity of one nucleotide pair from the normal gene. A variant is considered of a gene if it is within 100,000 base pairs of, preferably within 10,000 base pairs of, or more preferably contained in the transcribed sequence of the gene.

In a preferred embodiment, the ensemple of markers may comprise at least one, preferably at least two, and more preferably at least three SNP gene variants selected from the consisting of rs1041163 (VCAM1); rs1042718 (ADRB2); rs10460960 (CCK); rs10508244 (PFKP); rs10513055 (PIK3CB); rs10515070 (PIK3R1); rs107540 (CRHR2); rs10890819 (ACAT1); rs1131010 (PECAM1); rs1143634 (IL1B); rs11503016 (GABRA2); rs1171276 (LEPR); rs1255 (MDH1); rs1290443 (RARB); rs1322783 (DISC1); rs1356413 (PIK3CA); rs1396862 (CRHR1); rs1398176 (GABRA4); rs1440451 (HTR5A); rs167771 (DRD3); rs1799978 (DRD2); rs1800471 (TGFB1); rs1800871 (IL10); rs1801105 (HNMT); rs1801278 (IRS1); rs1801714 (ICAM1); rs1805002 (CCKBR); rs1891311 (HTR7); rs2005590 (APOL4); rs2067477 (CHRM1); rs2070424 (SOD1); rs2070586 (DAO); rs2076672 (APOL5); rs2162189 (SST); rs2229126 (ADRA1A); rs2240403 (CRHR2); rs2269935 (PFKM); rs2276307 (HTR3B); rs2278718 (MDH1); rs2296189 (FLT1); rs2298122 (DRD1IP); rs2514869 (ANGPT1); rs2515449 (MCPH1); rs322695 (RARB); rs324651 (CHRM2); rs334555 (GSK3B); rs3756007 (GABRA2); rs3760396 (CCL2); rs3822222 (CCKAR); rs3917550 (PON1); rs4121817 (PIK3C3); rs4149056 (SLCO1B1); rs4520 (APOC3); rs4531 (DBH); rs4675096 (IRS1); rs4726107 (PRKAG2); rs4792887 (CRHR1); rs4917348 (RXRA); rs4933200 (ANKRD1); rs5049 (AGT); rs5092 (APOA4); rs5361 (SELE); rs563895 (AVEN); rs5896 (F2); rs600728 (TEK); rs6078 (LIPC); rs6092 (SERPINE1); rs6131 (SELP); rs659734 (HTR2A); rs6700734 (TNFSF6); rs6967107 (WBSCR14); rs706713 (PIK3R1); rs707922 (APOM); rs7200210 (SLC12A4); rs722341 (ABCC8); rs7412 (APOE); rs7556371 (PIK3C2B); rs8178990 (CHAT); rs870995 (PIK3CA); rs885834 (CHAT); rs908867 (BDNF); rs936960 (LIPC); and combinations thereof.

In the foregoing list of SNPs, the abbreviation for the corresponding gene is provided in perentheses following each SNP. The specific variant will be selected from the foregoing SNPs or other variants of these or other genes determined to be associated with exercise response. Each individual gene variant is statistically associated to the respective physiological end point. The following table identifies exemplary SNPs, ranked based on the selection criteria of p≦0.05, for the physiological endpoints of change in blood LDL cholesterol level; change in blood HDL cholesterol level; change in log of blood triglyceride level; change in blood glucose level; change in LDL cholesterol, small fraction level; change in HDL cholesterol, large fraction level; change in systolic blood pressure; change in diastolic blood pressure; change in body mass; change in body mass index; change in waist size; change in fat percentage; change in weight normalized maximum oxygen uptake; and change in maximum oxygen uptake.

TABLE 1

| | | |
|---|---|---|
| SNP | Gene | p |

Change in LDL cholesterol (mg/dl)

| | | |
|---|---|---|
| rs2005590 | APOL4 | 0.000475 |
| rs3118536 | RXRA | 0.003988 |
| rs1041163 | VCAM1 | 0.007553 |
| rs334555 | GSK3B | 0.008841 |
| rs6960931 | PRKAG2 | 0.011511 |
| rs1800471 | TGFB1 | 0.011555 |
| rs1799978 | DRD2 | 0.011973 |
| rs707922 | APOM | 0.015471 |
| rs870995 | PIK3CA | 0.032487 |
| rs2162189 | SST | 0.042092 |
| rs5092 | APOA4 | 0.043301 |
| rs1398176 | GABRA4 | 0.046402 |
| rs2069827 | IL6 | 0.047419 |

Change in HDL cholesterol (mg/dl)

| | | |
|---|---|---|
| rs3760396 | CCL2 | 0.003401 |
| rs3791981 | APOB | 0.0093 |
| rs1143634 | IL1B | 0.010705 |
| rs10513055 | PIK3CB | 0.022683 |
| rs916829 | ABCC8 | 0.027108 |
| rs894251 | SCARB2 | 0.027512 |
| rs1891311 | HTR7 | 0.029401 |
| rs1800871 | IL10 | 0.031885 |
| rs521674 | ADRA2A | 0.039989 |
| rs5883 | CETP | 0.044562 |
| rs5049 | AGT | 0.046238 |

Change in Triglycerides (TG) (mg/dl) as log(TG)

| | | |
|---|---|---|
| rs26312 | GHRL | 0.005671 |
| rs7602 | LEPR | 0.008856 |
| rs11503016 | GABRA2 | 0.011189 |
| rs4890109 | RARA | 0.011345 |
| rs2070586 | DAO | 0.013713 |
| rs2278718 | MDH1 | 0.015428 |
| rs908867 | BDNF | 0.018318 |
| rs4121817 | PIK3C3 | 0.019589 |
| rs2240403 | CRHR2 | 0.020294 |
| rs722341 | ABCC8 | 0.021356 |
| rs4795180 | ACACA | 0.027138 |
| rs2276307 | HTR3B | 0.037126 |
| rs916829 | ABCC8 | 0.039972 |
| rs2162189 | SST | 0.042562 |
| rs563895 | AVEN | 0.045085 |
| rs1800871 | IL10 | 0.04633 |
| rs1171276 | LEPR | 0.047472 |
| rs10460960 | CCK | 0.049237 |

Change in blood glucose level (mg/dl)

| | | |
|---|---|---|
| rs322695 | RARB | 0.001533 |
| rs3822222 | CCKAR | 0.005801 |
| rs5361 | SELE | 0.013081 |
| rs737865 | TXNRD2 | 0.017054 |
| rs6131 | SELP | 0.018211 |
| rs722341 | ABCC8 | 0.021209 |
| rs10508244 | PFKP | 0.031791 |
| rs1042718 | ADRB2 | 0.032416 |
| rs2229126 | ADRA1A | 0.034765 |
| rs1800808 | SELP | 0.035979 |
| rs107540 | CRHR2 | 0.040179 |
| rs1322783 | DISC1 | 0.042759 |
| rs4531 | DBH | 0.043734 |
| rs2070424 | SOD1 | 0.044529 |
| rs10082776 | RARG | 0.044745 |
| rs2702285 | AVEN | 0.04787 |

Change in LDL cholesterol, small fraction (mg/dl)

| | | |
|---|---|---|
| rs2076672 | APOL5 | 0.003841 |
| rs5880 | CETP | 0.006477 |
| rs1150226 | HTR3A | 0.006515 |
| rs6131 | SELP | 0.01168 |
| rs4917348 | RXRA | 0.013288 |
| rs8192708 | PCK1 | 0.013336 |
| rs885834 | CHAT | 0.016769 |
| rs4675096 | IRS1 | 0.016883 |
| rs1131010 | PECAM1 | 0.018629 |
| rs6092 | SERPINE1 | 0.019999 |
| rs10515070 | PIK3R1 | 0.021004 |
| rs6078 | LIPC | 0.029452 |
| rs1805002 | CCKBR | 0.030311 |
| rs10890819 | ACAT1 | 0.030878 |
| rs659734 | HTR2A | 0.039957 |
| rs833060 | VEGF | 0.040981 |
| rs706713 | PIK3R1 | 0.04514 |
| rs2032582 | ABCB1 | 0.048449 |

Change in HDL cholesterol, large fraction (mg/dl)

| | | |
|---|---|---|
| rs1800871 | IL10 | 0.001492 |
| rs10513055 | PIK3CB | 0.00961 |
| rs4520 | APOC3 | 0.014903 |
| rs1042718 | ADRB2 | 0.01633 |
| rs5049 | AGT | 0.018933 |
| rs3760396 | CCL2 | 0.025747 |
| rs2020933 | SLC6A4 | 0.030597 |
| rs6586179 | LIPA | 0.037937 |
| rs3822222 | CCKAR | 0.046561 |

Change in Systolic Blood Pressure (SBP) (mmHg)

| | | |
|---|---|---|
| rs1801105 | HNMT | 0.01062 |
| rs597316 | CPT1A | 0.016046 |
| rs4149056 | SLCO1B1 | 0.01699 |
| rs6967107 | WBSCR14 | 0.017619 |
| rs7200210 | SLC12A4 | 0.019928 |
| rs10515070 | PIK3R1 | 0.022728 |
| rs706713 | PIK3R1 | 0.032316 |
| rs1800871 | IL10 | 0.03233 |
| rs4726107 | PRKAG2 | 0.034068 |
| rs2298122 | DRD1IP | 0.035164 |
| rs5896 | F2 | 0.039114 |
| rs2070424 | SOD1 | 0.041442 |
| rs8178990 | CHAT | 0.041897 |
| rs1805002 | CCKBR | 0.049848 |

Change in Diastolic Blood Pressure (DBP) (mmHg)

| | | |
|---|---|---|
| rs3762272 | PKLR | 0.002134 |
| rs722341 | ABCC8 | 0.002567 |
| rs1556478 | LIPA | 0.01054 |
| rs2067477 | CHRM1 | 0.015146 |
| rs4531 | DBH | 0.017324 |
| rs7556371 | PIK3C2B | 0.02814 |
| rs2702285 | AVEN | 0.028454 |
| rs1438732 | NR3C1 | 0.0307 |
| rs2228502 | CPT1A | 0.033767 |
| rs3853188 | SCARB2 | 0.038147 |
| rs6837793 | NPY5R | 0.038438 |
| rs324651 | CHRM2 | 0.044854 |

Change in Body Mass (BMS) (Kg)

| | | |
|---|---|---|
| rs1801278 | IRS1 | 0.000737 |
| rs3756007 | GABRA2 | 0.002309 |
| rs2070424 | SOD1 | 0.007473 |
| rs676643 | HTR1D | 0.013193 |
| rs870995 | PIK3CA | 0.018349 |
| rs2807071 | OAT | 0.019668 |
| rs10508244 | PFKP | 0.022159 |
| rs2162189 | SST | 0.022405 |
| rs4792887 | CRHR1 | 0.027628 |
| rs2296189 | FLT1 | 0.035579 |
| rs6700734 | TNFSF6 | 0.038472 |
| rs1255 | MDH1 | 0.039926 |
| rs1440451 | HTR5A | 0.04227 |
| rs3769671 | POMC | 0.047085 |
| rs722341 | ABCC8 | 0.048351 |
| rs1041163 | VCAM1 | 0.048917 |
| rs2742115 | OLR1 | 0.049709 |

Change in Body Mass Index (BMI) (kg/m²)

| | | |
|---|---|---|
| rs1801278 | IRS1 | 0.000659 |
| rs3756007 | GABRA2 | 0.001644 |
| rs676643 | HTR1D | 0.007354 |
| rs2070424 | SOD1 | 0.007555 |
| rs870995 | PIK3CA | 0.011284 |
| rs2807071 | OAT | 0.021223 |
| rs2162189 | SST | 0.021334 |
| rs1440451 | HTR5A | 0.024347 |
| rs10508244 | PFKP | 0.027131 |
| rs4792887 | CRHR1 | 0.036982 |
| rs3769671 | POMC | 0.039796 |
| rs167771 | DRD3 | 0.039883 |
| rs936960 | LIPC | 0.045164 |
| rs2296189 | FLT1 | 0.046396 |

Change in Waist Size

| | | |
|---|---|---|
| rs6700734 | TNFSF6 | 0.010206 |
| rs2269935 | PFKM | 0.012618 |
| rs4933200 | ANKRD1 | 0.018679 |
| rs10082776 | RARG | 0.023572 |
| rs1935349 | HTR7 | 0.033396 |
| rs2514869 | ANGPT1 | 0.035904 |
| rs2020933 | SLC6A4 | 0.044248 |

Change in Percent Fat

| | | |
|---|---|---|
| rs600728 | TEK | 0.001596 |
| rs8178990 | CHAT | 0.013731 |
| rs1290443 | RARB | 0.019679 |
| rs722341 | ABCC8 | 0.038435 |
| rs885834 | CHAT | 0.045965 |
| rs2162189 | SST | 0.046065 |
| rs2070424 | SOD1 | 0.049694 |

Change in maximum oxygen uptake, weight normalized
(mL/kg/min) (Vmax)

| | | |
|---|---|---|
| rs4149056 | SLCO1B1 | 0.00075 |
| rs2298122 | DRD1IP | 0.001981 |
| rs563895 | AVEN | 0.003272 |
| rs7412 | APOE | 0.009196 |
| rs2702285 | AVEN | 0.0125 |
| rs5896 | F2 | 0.014676 |
| rs1356413 | PIK3CA | 0.015499 |
| rs3917550 | PON1 | 0.015993 |
| rs662 | PON1 | 0.01665 |
| rs10460960 | CCK | 0.023304 |
| rs7520974 | CHRM3 | 0.02476 |
| rs1396862 | CRHR1 | 0.029987 |
| rs1801714 | ICAM1 | 0.035731 |
| rs8178990 | CHAT | 0.040374 |
| rs1800871 | IL10 | 0.042156 |
| rs334555 | GSK3B | 0.042954 |
| rs2296189 | FLT1 | 0.04367 |
| rs6809631 | PPARG | 0.046208 |

Change in maximum oxygen uptake (L/min) (Vmaxl)

| | | |
|---|---|---|
| rs5896 | F2 | 0.005554 |
| rs334555 | GSK3B | 0.005953 |
| rs4149056 | SLCO1B1 | 0.007495 |
| rs563895 | AVEN | 0.009217 |
| rs4072032 | PECAM1 | 0.012859 |
| rs722341 | ABCC8 | 0.016688 |
| rs2515449 | MCPH1 | 0.025517 |
| rs1805002 | CCKBR | 0.03223 |
| rs2298122 | DRD1IP | 0.044082 |
| rs7412 | APOE | 0.045224 |
| rs1396862 | CRHR1 | 0.049309 |

The SNPs and genes in Table 1 are provided in the nomenclature adopted by the National Center for Biotechnology Information (NCBI) of the National Institute of Health. The sequence data for the SNPs and genes listed in Table 1 is known in the art and is readily available from the NCBI dbSNP and GenBank databases. The sequence information for these and other representative SNPs is provided below in Table 2.

TABLE 2

| | | |
|---|---|---|
| | SEQ | |
| SNP | ID | Sequence |

| | | |
|---|---|---|
| rs2005590 | 1 | CACCACCTGGAAAAATCATGCTCAT[C/ |
| | | T]GTTCAGTGACAAAATCAGGCATTGC |
| rs10082776 | 2 | GAGGTCCCAAGGTGAATGATGGTCT[A/ |
| | | G]AGGACTTCTGGTGGAGAGAACTCCT |
| rs1041163 | 3 | AAGCTAGTATTTCCTGAATCAATTT[C/ |
| | | T]TCTGATCCCTAGATATTTGGTAGGT |
| rs1042718 | 4 | CTTGCCCATTCAGATGCACTGGTAC[A/ |
| | | C]GGGCCACCCACCAGGAAGCCATCAA |
| rs1045642 | 5 | GCCGGGTGGTGTCACAGGAAGAGAT[A/C/G/ |
| | | T]GTGAGGGCAGCAAAGGAGGCCAACA |
| rs10460960 | 6 | CAGGCCATACTGAAAATGCTAGTCC[A/ |
| | | G]CCAAGCACACTTTGAGATCATTTCT |
| rs10508244 | 7 | GTGTACATTTGAGTGTGAGGTAGTA[C/ |
| | | T]GTTTCTGCATGTTAGTGTGTGCATG |
| rs10513055 | 8 | TGCTGGGTAGGAAATTAAGTGAATA[A/ |
| | | C]TTTTTGTGATCCAAGAAAGAGATTT |
| rs10515070 | 9 | TGAGAGATTCCTCCCTGTACGATAG[A/ |
| | | T]GTCTTACTTTTCCACTTTGCTTGTA |
| rs1064344 | 10 | TAGGTGTGGTATCTTTACTGGAACC[A/ |
| | | G]ATAAATGCACCTCTGGCTCTTGATA |
| rs107540 | 11 | GGTTAGGGACTGGAGCCTGCTGCCC[A/ |
| | | G]GCACGGTGGTCACACCCTGGCCAGC |
| rs10890819 | 12 | GGTGAGAACAAAGTGAGGGGCGATA[C/ |
| | | T]TCCATTATGCTAGCTTCTGGTTTGC |
| rs11100494 | 13 | GTCACAGAAAGATGTCATCATCCAG[A/ |
| | | C]ATTGCGTCCACACAGTCAACAGTAG |
| rs1131010 | 14 | GTGTTGCAGATAATTGCCATTCCCA[C/ |
| | | T]GCCAAAATGTTAAGTGAGGTTCTGA |
| rs1143634 | 15 | TCCACATTTCAGAACCTATCTTCTT[C/ |
| | | T]GACACATGGGATAACGAGGCTTATG |
| rs1150226 | 16 | CAGGCAGGAGCAGGAAGACCATTCT[C/ |
| | | T]TTACTCCCCAGGGTGACATAACCAA |
| rs11503016 | 17 | TTAGTCTACTCAAATACATGGATAG[A/ |
| | | T]TAAAGATGTTTGGATCTATGGTTTC |
| rs1171276 | 18 | TAAAAGTTTCATGTACATTAAATAT[A/ |
| | | G]AATTTCTTTTGGCTGGAAATGGCAT |
| rs1255 | 19 | CTCACGAACAAGGACGCTTTGAAGA[A/ |
| | | G]GTGGAATTACTGTGCAAGGAGTACT |
| rs1290443 | 20 | GTAGAGAAGCTCTTTCATGTTGTCA[A/ |
| | | G]TTTTAGAAATCCAAATCATTAGAGA |
| rs1322783 | 21 | CTGCTAGAAATGCCAGAAAATGTAA[C/ |
| | | T]AGATGCTAGAAGAGGAGTGATTACT |
| rs132642 | 22 | CAGCCAGGTCACTGAGAGACTTTCC[A/ |
| | | T]TGGAGCTCTCCAGTCACTGACCTGA |
| rs1355920 | 23 | GGATATCAACTGAGGAAGATAATAA[A/ |
| | | G]CTATAAAAAGATGAAAAGGAAAGGC |
| rs1356413 | 24 | AGTGAACTATTAATAATTATAGAAG[C/ |
| | | G]ATATAGAGGCATATGTCTAAAAAGA |
| rs1396862 | 25 | AGCTTGGTTTTAGGAAAAAGCACCT[C/ |
| | | T]TGCAGTTCAGAAGCCCTGGTCCAAC |
| rs1398176 | 26 | ACTGCATCCTTTTACTTACCCCACA[C/ |
| | | T]TGGGCTGCATTCTTTTTATTTTACT |
| rs1440451 | 27 | AGCCCTTGTTCATGATGAGATTATA[C/ |
| | | G]CTGATCTGACGTGAGAATGCCTACA |
| rs1468271 | 28 | AAATGACCCTGTAATTTTCAGAAAC[A/ |
| | | GICACATAGGAGTGGGTGTCTGTGGTG |
| rs1478290 | 29 | CCTCCAGGCTTCCCCTCATTCATTA[G/ |
| | | T]GCTTTTGGCTTCAGCCACATTGGTC |
| rs1556478 | 30 | GAGTCACGGAGACTTATGCACCAGA[A/ |
| | | G]TGAAATGCTGAGATGTTCTTGGGCT |
| rs167771 | 31 | CTCATGCTCCAAAGTCTATCACAAT[A/ |
| | | G]ATCCTCTTTTCCATAAAGCCCTTTC |
| rs1799978 | 32 | AGGACCCAGCCTGCAATCACAGCTT[A/ |
| | | G]TTACTCTGGGTGTGGGTGGGAGCGC |
| rs1800471 | 33 | TGGCTACTGGTGCTGACGCCTGGCC[C/ |
| | | G]GCCGGCCGCGGGACTATCCACCTGC |
| rs800545 | 34 | TATTAGGAGCTCGGAGCAAGAAGGC[A/ |
| | | G]CCCACCGAGAGCGTCTGAAGCGCGA |
| rs1800871 | 35 | GTGTACCCTTGTACAGGTGATGTAA[C/ |
| | | T]ATCTCTGTGCCTCAGTTTGCTCACT |
| rs1801105 | 36 | AAATACAAAGAGCTTGTAGCCAAGA[C/ |
| | | T]ATCGAACCTCGAGAACGTAAAGTTT |
| rs1801278 | 37 | GGGCAGACTGGGCCCTGCACCTCCC[A/ |
| | | G]GGGCTGCTAGCATTTGCAGGCCTAC |
| rs1801714 | 38 | GGGGTTCCAGCCCAGCCACTGGGCC[C/ |
| | | T]GAGGGCCCAGCTCCTGCTGAAGGCC |
| rs1805002 | 39 | CATGGGCACATTCATCTTTGGCACC[A/ |
| | | G]TCATCTGCAAGGCGGTTTCCTACCT |
| rs1877394 | 40 | ACCGAGTTTGAGACGTGGGTGAAAC[A/ |
| | | G]TAGGTGGAAAAGTCCAGCAAGAAGG |
| rs1891311 | 41 | AAGAAATGACCGGTTATACTCTTCT[A/ |
| | | G]TAAAGGAATCCTGGAGGTGTATGTT |
| rs1951795 | 42 | GTTGACTTATTTCAGTGGTTCAAAA[A/ |
| | | C]ATTTCTTCAACGCTTAACCATGACT |
| rs2005590 | 43 | CACCACCTGGAAAAATCATGCTCAT[C/ |
| | | T]GTTCAGTGACAAAATCAGGCATTGC |
| rs2020933 | 44 | TCAGTTTTGTCCAGAAAAGTGAACC[A/ |
| | | T]GGTCAATGGATTATTTATGAGCCTG |
| rs2033447 | 45 | ATGAGGAACTTTGTCATGTTCACTG[C/ |
| | | T]TGTATCTCTAGCACCCGGCATAGGG |
| rs2049045 | 46 | ACCAAAATCTCTCTTCTTCGATAAA[C/ |
| | | G]TTCCCAGGAGGTAACCCAATTTCTA |
| rs2058112 | 47 | GCTGTAGGATTTCTCCAAGGGCTTT[C/ |
| | | T]GAAGTATGTAGGGCAAGAAGAAACA |
| rs2067477 | 48 | TCTATACCACGTACCTGCTCATGGG[A/ |
| | | C]CACTGGGCTCTGGGCACGCTGGCTT |
| rs2070424 | 49 | GGGACATAGCTTTGTTAGCTATGCC[A/ |
| | | G]GTAATTAACAGGCATAACTCAGTAA |
| rs2070586 | 50 | CGAGTTGCCAGGAGCTGAGGTCTGC[A/ |
| | | G]GGAGGAGAGTTGTGAGTGAAGATGA |
| rs2076672 | 51 | AAGCACCTGGAGGATGGGGCAAGGA[C/ |
| | | T]GGAGACAGCAGAGGAACTGAGAGCA |
| rs2162189 | 52 | CACCTCTAGAAGGCATCCAGGCCTC[A/ |
| | | G]CCTCTTTCATGTGCAGCTTTTTCTG |
| rs2229126 | 53 | TCTCCCTCAGTGAGAACGGGGAGGA[A/ |
| | | T]GTCTAGGACAGGAAAGATGCAGAGG |
| rs2240403 | 54 | ATCTGGTCACAGGCCCCACCTGGAA[C/ |
| | | T]GACTGCAGGAAGGAGTTGAAATAGA |
| rs2276307 | 55 | TTGGCCTTCTCTCTTGGGCCAAGGA[A/ |
| | | G]TTTCTGCTCTATTGCATGTTCTCAT |
| rs2278718 | 56 | TCCCCTCCCTAGAGTTACACACGCT[A/ |
| | | C]TCTCTCCCGCCAATTGCCGGGCTCC |
| rs2296189 | 57 | TGTAGATTTTGTCAAAGATAGATTC[A/ |
| | | G]GGAGCCATCCATTTCAGAGGAAGTC |
| rs2298122 | 58 | GTAGGCAGCTGGCAGGGACCCAAGA[G/ |
| | | T]AGCCCTGAACTGAGAGGGGAGGGAG |
| rs2430683 | 59 | TTGGATTTTGGCATCTTTGGGATCC[G/ |
| | | T]TGGTAGCCTGGTGTTTGCTGGTTAC |
| rs2471857 | 60 | TTTTCTTCCCAGTTGCACTAACAGA[A/ |
| | | G]CCTTTGATTCAGTTCAGCAAACATC |
| rs2515449 | 61 | TCCTAATTTCAACTTATAAACATAC[A/ |
| | | G]TTGCTATAAATATGTTCAATGAAGA |
| rs26312 | 62 | ATGTGCTGTTGCTGCTCTGGCCTCT[A/ |
| | | G]TGAGCCCCGGGAGTCCGCAGGGAGC |
| rs2702285 | 63 | AAACAGCTTTCAAATGTCATGCATT[A/ |
| | | G]TGTGGCAGGAGTAGGTTTTAAATAT |
| rs2740574 | 64 | GAGGACAGCCATAGAGACAAGGGCA[A/ |
| | | C/G/T]GAGAGAGGCGATTTAATAGATTTTA |
| rs2807071 | 65 | CAACAGTCAAACTACATCTTCTCAA[C/ |
| | | T]TAATTGCTAGTCTCCCTAACCAAAA |
| rs3024492 | 66 | GCTGTAAATGAGGAAAGACTCCTGG[A/ |
| | | T]GTCAGATCTCTTGCTCATTTCTCTT |
| rs3118536 | 67 | GGGTCTGCAGGTGCACGGTTTCCTG[A/ |
| | | C]TTGCCCAGGTGTCTCTGAGCCTGTC |
| rs322695 | 68 | CTGCCCTGTAGGATTGTGTTCCTCT[A/ |
| | | G]AAACTGTCCCCTAAATTATGGTGCC |
| rs324651 | 69 | ATTTAATTCAATTTATCAGTATTAT[G/ |
| | | T]CTAAGTTTCATGGATTGATGAGATA |
| rs334555 | 70 | ATGTAATTATATCTTATTATTAAAA[C/ |
| | | G]TCTACCAACTCAAAGCTTCCCCCTT |
| rs3750546 | 71 | GGCTCCTGAGGATGAAGGGGCGTCC[A/ |
| | | G]TGGCCAGGCAGCAGTGAGAACTCCA |
| rs3756007 | 72 | ACACTGTTTTGCGCACACGTAATAA[C/ |
| | | T]AACACCCTGGACTTTAAACTGGCAT |
| rs3760396 | 73 | GTGTACAAGTCCTCCAACTAGTTGC[C/ |
| | | G]TGCTTGGGTCCTCTCTCTGTCCTCA |
| rs3762272 | 74 | CTGGAACAAAGATTCTCCTTTCCTC[A/ |
| | | G]TTCACCACTTTCTTGCTGTTCTGGG |
| rs3822222 | 75 | ACGTTCCCCACAAGTCGGTCCCCAT[C/ |
| | | T]ATCCATGTTGGAGGTCAGTTTCTAA |
| rs3917550 | 76 | CCCTAAGAAAGCAGCCCTCTACCTC[C/ |
| | | T]GAAAAACAGCAAGACGTTGCTTTCC |
| rs4072032 | 77 | CCCTAAGAAAGCAGCCCTCTACCTC[C/ |
| | | T]GAAAAACAGCAAGACGTTGCTTTCC |
| rs4121817 | 78 | TGAGCAGCACTCCGAATGAAGGCTG[A/ |
| | | G]CAGTGAAACTGAATGACTTATACCT |
| rs4149056 | 79 | TCTGGGTCATACATGTGGATATATG[C/ |
| | | T]GTTCATGGGTAATATGCTTCGTGGA |
| rs4244285 | 80 | TTCCCACTATCATTGATTATTTCCC[A/ |
| | | G]GGAACCCATAACAAATTACTTAAAA |
| rs4520 | 81 | CCTCCCTTCTCAGCTTCATGCAGGG[C/ |
| | | T]TACATGAAGCACGCCACCAAGACCG |
| rs4531 | 82 | TTACTACCCAGAGGAAGCCGGCCTT[G/ |
| | | T]CCTTCGGGGGTCCAGGGTCCTCCAG |
| rs4675096 | 83 | TGTTAGTGTTTTCCAAGGTGTGATT[A/ |
| | | G]AAAATGGAGATTTCTTACCTCATCC |
| rs4680 | 84 | CCAGCGGATGGTGGATTTCGCTGGC[A/ |
| | | G]TGAAGGACAAGGTGTGCATGCCTGA |
| rs4726107 | 85 | GTTAGAAGTAGAAAAGGGGAGGGGG[C/ |
| | | T]AGTATTTAGCCTCTGTCCCCACTAA |
| rs4792887 | 86 | CCTCTGGGGTCACCAGGTACATCTT[C/ |
| | | T]GATCTTGGCCACACTGGAGAGTCAA |
| rs4890109 | 87 | CTGGCAGCTCTCTGTCAGGCTGGGG[G/ |
| | | T]TGGACGAGGCCCTGAGCAGCCTGCA |
| rs4917348 | 88 | CCGGGGTGGGGTTAGAGGGGATGGT[A/ |
| | | G]CCTGGCAGTGTGCAGCAGACTGGCA |
| rs5049 | 89 | TAAATGTGTAACTCGACCCTGCACC[A/ |
| | | G]GCTCACTCTGTTCAGCAGTGAAACT |
| rs5092 | 90 | AGGTCAGTGCTGACCAGGTGGCCAC[A/ |
| | | G]GTGATGTGGGACTACTTCAGCCAGC |
| rs521674 | 91 | AATATTCTACTCCCTCTTCCCCTTA[A/ |
| | | T]TGAAGGATGCTGTGTGTACATCTGA |
| rs5361 | 92 | AGCTGCCTGTACCAATACATCCTGC[A/ |
| | | C]GTGGCCACGGTGAATGTGTAGAGAC |
| rs5447 | 93 | CTTACTGGTTGGGAGCCTTCCCGAC[A/ |
| | | G]TGAACAAGATGCTGGATAAGGAAGA |
| rs563895 | 94 | TAGGGTAGAACAGGTTGGAGAAGGG[C/ |
| | | T]GGAGGATAAATCTGCATTGGCACAT |
| rs5880 | 95 | CCAGGATATCGTGACTACCGTCCAG[C/ |
| | | G]CCTCCTATTCTAAGAAAAGCTCTTC |
| rs5896 | 96 | TGCCGCAACCCCGACAGCAGCACCA[C/ |
| | | T]GGGACCCTGGTGCTACACTACAGAC |
| rs597316 | 97 | TGATCCATTTACGCGGCCCCCATTG[C/ |
| | | G]ACAATTAGGGCCTCCTCCCCGCCCC |
| rs600728 | 98 | CAGAGGCTCCACGACAATGAGTACA[A/ |
| | | G]CTGTGGTCCGTGGCTTCTTGAAAGA |
| rs6032470 | 99 | CTGCAAATGTTTGTTAAGCCTCTAC[C/ |
| | | T]GTTCCGGTAAGGACTGGGGCTAGAG |
| rs6078 | 100 | TCTGTCCCCTCCTCAGGTGGACGGC[A/ |
| | | G]TGCTAGAAAACTGGATCTGGCAGAT |
| rs6092 | 101 | CCTCACCTGCCTAGTCCTGGGCCTG[A/ |
| | | G]CCCTTGTCTTTGGTGAAGGGTCTGC |
| rs6131 | 102 | CAGTGTCAGCACCTGGAAGCCCCCA[A/ |
| | | G]TGAAGGAACCATGGACTGTGTTCAT |
| rs619698 | 103 | TGCTTGGGACAGGTGCGCTCCCAGA[A/ |
| | | C]GGGATCCTGTCGCCAGTTCTGGGGG |
| rs6312 | 104 | GAATAACAAATGTATCTCATGTGTG[A/ |
| | | G]ACCCTGAAGACAAATGTAAGTTCTC |
| rs6541017 | 105 | TATGTTTCCCTCTACTCAGTTATCC[A/ |
| | | G]ATTATTCATGACTAGATGAGATTAG |
| rs6586179 | 106 | GGATCCACAGCTGTCAGTTTCCCTC[C/ |
| | | T]AGACCCCTCAGAATGCAGGGTCCAG |
| rs659734 | 107 | GAATCTAGCTGCTTTCCGTTTATGA[C/ |
| | | T]TTCAGTTCAATTTCCTACCAGCTAT |
| rs660339 | 108 | AGTCAGGGGCCAGTGCGCGCTACAG[C/ |
| | | T]CAGCGCCCAGTACCGCGGTGTGATG |
| rs662 | 109 | CACTATTTTCTTGACCCCTACTTAC[A/ |
| | | G]ATCCTGGGAGATGTATTTGGGTTTA |
| rs6700734 | 110 | ACCCAAATAAACCAGAAATTGGTAA[A/ |
| | | G]TCATCACATGGAAATCAAATCAGTA |
| rs676643 | 111 | TCCCAGGTTCATCTTGACGCATCCT[A/ |
| | | G]AGCTACTTAACTTCGGTTCCTATCC |
| rs6837793 | 112 | TACCATGAATTGTCACTCAGAAGAA[A/ |
| | | G]CTTAATAGGCATTAATACTACACGA |
| rs6960931 | 113 | CCCCACTACCCCCACCACACTTGGC[C/ |
| | | T]GTGTGCCTTGCATTTCCCAGAAGTG |
| rs6967107 | 114 | CCCCACTACCCCCACCACACTTGGC[C/ |
| | | T]GTGTGCCTTGCATTTCCCAGAAGTG |
| rs706713 | 115 | AAAGGGGGGACTTTCCGGGAACTTA[C/ |
| | | T]GTAGAATATATTGGAAGGAAAAAAA |
| rs707922 | 116 | TAATCCTGTTTTATGAGATTTTAAC[A/ |
| | | C]CCTTACCTTGATTCCTAGGAGTCAA |
| rs7200210 | 117 | GTTTCAAGAGCTCCCTACCCAGGAA[A/ |
| | | G]CCCAAGCCTCACCCAGAATGAGGCT |
| rs722341 | 118 | TCATTAACATTAGTCATGTGGGAGA[C/ |
| | | T]AGGAGAAGAAGCTCTGCAGAAAAGG |
| rs737865 | 119 | AATAAAAAGCAACAGGACACAAAAA[C/ |
| | | T]CCCTGGCTGGAAAAATCCAAAAAGC |
| rs7412 | 120 | CCGCGATGCCGATGACCTGCAGAAG[C/ |
| | | T]GCCTGGCAGTGTACCAGGCCGGGGC |
| rs7556371 | 121 | AAAGCCGTGCTCTTAACCATCTGCC[A/ |
| | | G]AACTTGCACTGCCAGTCATTTGATA |
| rs7602 | 122 | TGTGCTTGGAGAGGCAGATAACGCT[A/ |
| | | G]AAGCAGGCCTCTCATGACCCAGGAA |
| rs8178990 | 123 | TGCAGCCAGCCTCATCTCTGGTGTA[C/ |
| | | T]TCAGCTACAAGGCCCTGCTGGACAG |
| rs8190586 | 124 | CCCCCACCCGCCATCAATCCTGCCG[A/ |
| | | G]CTCTGGCCGCTCTGCCTCATTCTCT |
| rs8192708 | 125 | CAATAAAGAATCTTGTCCCCAACAG[A/ |
| | | G]TTCTGGGTATAACCAACCCTGAGGG |
| rs870995 | 126 | ACCTTCAGGTATTAGCACTTGAAAT[A/ |
| | | C]TAACTTCTTTATGAAGCTCCTTATT |
| rs885834 | 127 | GAGCACGACGCCGTGCCGGGAATAG[A/ |
| | | G]GAAGCAGTGTGAGGACCACAAGACA |
| rs894251 | 128 | CATAGAAATCAAAGGGCAAGAACCA[C/ |
| | | T]GGCACAGTAAGGCCTCCTGAGAGGA |
| rs908867 | 129 | TCAGGCACCTACACCAACAATTCAG[A/ |
| | | G]GTATCCCACTGTAAGATATAATTTT |
| rs936960 | 130 | GGTGCAGAGCACGAGGCTGATTTTC[A/ |
| | | C]ATCCCAGTGTGGGCCACACCCTATG |

By combining the effect of several SNPs the necessary sensitivity and specificity of prediction is achieved for the ensemble of alleles, since the association of an individual SNP with the outcome does not have sufficient predictive power. The physigenomics method mathematically assigns to each SNP a coefficient according to pre-established rules and covariates. The generation of the coefficients is discussed in detail in the examples and in U.S. patent application Ser. No. 11/371,511 and U.S. patent application Ser. No. 11/010,716, both of which are incorporated by reference herein. The coefficient for each SNP may be either positive, indicating that the presence of that marker contributes to physiological response, or negative (i.e., a torpid marker). The most powerful predictions are achieved for a particular physiological endpoint by using SNPs having positive coefficients and SNPS having negative coefficients.

In accordance with this embodiment of the invention, the ensemble of marker genes comprises at least two SNPs, the presence of which in a human correlates with at least one physiological response to exercise; wherein the physiological response is selected from the group consisting of log of blood triglyceride level; blood LDL cholesterol level; blood HDL cholesterol level; ratio of total cholesterol to HDL cholesterol; LDL cholesterol, small fraction level; HDL cholesterol, large fraction level; blood glucose level; systolic blood pressure; diastolic blood pressure; body mass; body mass index; fat percentage; weight normalized maximum oxygen uptake; maximum oxygen uptake; and combinations thereof; and wherein the at least two SNP gene variants comprise at least one SNP gene variant having a positive coefficient and at least one SNP gene variant having a negative coefficient in the phyiotype model, including:

(1) in the case where said physiological response is a change in blood LDL cholesterol level, the marker set comprises: (i) at least one SNP gene variant having a positive coefficient selected from the group consisting of rs334555, rs1799978, rs870995, rs1398176, and rs5092; and (ii) at least one SNP gene variant having a negative coefficient selected from the group consisting of rs3118536, rs2005590, rs1041163, rs1800471, and rs707922; and

(2) in the case where the physiological response is a change in blood HDL cholesterol level, the marker set comprises: (i) at least one SNP gene variant having a positive coefficient selected from the group consisting of rs660339, rs894251, rs3760396, rs10513055, rs10513055, rs1800871, rs3760396, and rs1891311; and (ii) at least one SNP gene variant having a negative coefficient selected from the group consisting of rs936960, rs1143634, rs5049, and rs1891311; and

(3) in the case where the physiological response is a change in log of blood triglyceride level, the marker set comprises: (i) at least one SNP gene variant having a positive coefficient selected from the group consisting of rs722341, rs7602, rs4121817, rs5880, rs908867, rs2278718, rs2240403, and rs1171276; and (ii) at least one SNP gene variant having a negative coefficient selected from the group consisting of rs563895, rs2070586, rs1800871, rs2070586, rs10460960, rs2276307, rs11503016, and rs563895; and

(4) in the case where the physiological response is a change in blood glucose level, the marker set comprises: (i) at least one SNP gene variant having a positive coefficient selected from the group consisting of rs737865, rs10082776, rs10508244, rs1322783, rs2070424, rs107540, rs1042718, rs5361, and rs322695; and (ii) at least one SNP gene variant having a negative coefficient selected from the group consisting of rs1398176, rs722341, rs3822222, and rs2229126; and

(5) in the case where the physiological response is a change in LDL cholesterol, small fraction level, the marker set comprises: (i) at least one SNP gene variant having a positive coefficient selected from the group consisting of rs2033447, rs1877394, rs4917348, rs1131010, rs706713, rs4675096, and rs4917348; and (ii) at least one SNP gene variant having a negative coefficient selected from the group consisting of rs1045642, rs6131, rs2076672, rs6092, rs6078, rs659734, and rs885834; and

(6) in the case where the physiological response is a change in HDL cholesterol, large fraction level, the marker set comprises: (i) at least one SNP gene variant having a positive coefficient selected from the group consisting of rs10513055, rs1800871, and rs3760396; and (ii) at least one SNP gene variant having a negative coefficient selected from the group consisting of rs1799978, rs8192708, rs521674, rs5049, rs1042718, and rs4520; and

(7) in the case where the physiological response is a change in systolic blood pressure, the marker set comprises: (i) at least one SNP gene variant having a positive coefficient selected from the group consisting of rs597316, rs10515070, rs4149056, rs2298122, and rs6967107; and (ii) at least one SNP gene variant having a negative coefficient selected from the group consisting of rs2070424, rs6586179, rs1064344, rs1100494, rs1800871, rs1801105, rs7200210, and rs4726107; and

(8) in the case where the physiological response is a change in diastolic blood pressure, the marker set comprises: (i) at least one SNP gene variant having a positive coefficient selected from the group consisting of rs722341, rs3762272, rs600728, rs7556371, rs4531, and rs2067477; and (ii) at least one SNP gene variant having a negative coefficient selected from the group consisting of rs660339, rs662, rs2162189, rs2702285, and rs324651.

(9) in the case where the physiological response is a change in body mass, the marker set comprises: (i) at least one SNP gene variant having a positive coefficient selected from the group consisting of rs870995, rs600728, rs676643, rs2070424, rs1801278, rs6700734, and rs4792887; and (ii) at least one SNP gene variant having a negative coefficient selected from the group consisting of rs6541017, rs1041163, rs722341, rs2162189, rs1255, rs1440451, and rs3756007; and

(10) in the case where the physiological response is a change in body mass index, the marker set comprises: (i) at least one SNP gene variant having a positive coefficient selected from the group consisting of rs5880, rs600728, rs676643, rs2070424, rs1801278, and rs4792887; and (ii) at least one SNP gene variant having a negative coefficient selected from the group consisting of rs132642, rs2162189, rs1440451, rs936960, and rs167771; and

(11) in the case where the physiological response is a change in percentage fat, the marker set comprises: (i) at least one SNP gene variant having a positive coefficient selected from the group consisting of rs676643, rs2070424, rs885834, rs8178990, and rs600728; and (ii) at least one SNP gene variant having a negative coefficient selected from the group consisting of rs8192708, rs6312, rs722341, and rs1290443; and

(12) in the case where the physiological response is a change in weight normalized maximum oxygen uptake, the marker set comprises: (i) at least one SNP gene variant having a positive coefficient selected from the group consisting of rs8178990, rs5447, rs1800871, rs4149056, rs7412, and rs1901714; and (ii) at least one SNP gene variant having a negative coefficient selected from the group consisting of rs2298122, rs26312, rs563895, rs5896, rs3917550, rs2296189, and rs1356413; and

(13) in the case where the physiological response is a change in maximum oxygen uptake, the marker set comprises: (i) at least one SNP gene variant having a positive coefficient selected from the group consisting of rs11503016, rs2515449, rs334555, rs722341, rs4149056, rs7412, rs1396862, rs2515449, and rs1805002; and (ii) at least one SNP gene variant having a negative coefficient selected from the group consisting of rs597316, rs26312, rs2020933, rs563895, and rs5896.

The SNPs may be provided as an array on a solid support or the like. The array may be a micro or nano array. These SNPS may be used in a method of predicting an individual's physiological response to exercise. The method generally comprises (1) obtaining genetic material from the individual; and (2) assaying the genetic material for the presence of the at least two SNP gene variants of the foregoing ensemble.

In other interesting embodiments of the invention, the marker gene set correlated with physiological response to exercise comprises the plurality of SNP gene variants listed below (a)-(m), each being a distinct embodiment of the invention:

(a) The physiological response is a change in blood LDL cholesterol level and the plurality of SNP gene variants comprise at least one single SNP gene variant selected from the group consisting of rs334555, rs1799978, rs870995, rs1398176, rs5092, rs3118536, rs2005590, rs1041163, rs1800471, rs707922, and combinations thereof.

(b) The physiological response is a change in blood HDL cholesterol level and the plurality of SNP gene variants comprise at least one SNP gene variant selected from the group consisting of rs660339, rs894251, rs3760396, rs10513055, rs10513055, rs1800871, rs3760396, rs1891311, rs936960, rs1143634, rs5049, rs1891311, and combinations thereof.

(c) The physiological response is a change in log of blood triglyceride level and the plurality of SNP gene variants comprise at least one SNP gene variant selected from the group consisting of rs722341, rs7602, rs4121817, rs5880, rs908867, rs2278718, rs2240403, rs1171276, rs563895, rs2070586, rs1800871, rs2070586, rs10460960, rs2276307, rs11503016, and rs563895, and combinations thereof.

(d) The physiological response is a change in blood glucose level and the plurality of SNP gene variants comprise at least one SNP gene variant selected from the group consisting of rs737865, rs10082776, rs10508244, rs1322783, rs2070424, rs107540, rs1042718, rs5361, rs322695, rs1398176, rs722341, rs3822222, rs2229126, and combinations thereof.

(e) The physiological response is a change in LDL cholesterol, small fraction level and the plurality of SNP gene variants comprise at least one SNP gene variant selected from the group consisting of rs2033447, rs1877394, rs4917348, rs1131010, rs706713, rs4675096, rs4917348, rs1045642, rs6131, rs2076672, rs6092, rs6078, rs659734, rs885834, and combinations thereof.

(f) The physiological response is a change in LDL cholesterol, large fraction level and the plurality of SNP gene variants comprise at least one SNP gene variant selected from the group consisting of rs10513055, rs1800871, rs3760396, rs1799978, rs8192708, rs521674, rs5049, rs1042718, rs4520, and combinations thereof.

(g) The physiological response is a change in systolic blood pressure and the plurality of SNP gene variants comprise at least one SNP gene variant selected from the group consisting of rs597316, rs10515070, rs4149056, rs2298122, rs6967107, rs2070424, rs6586179, rs1064344, rs1100494, rs1800871, rs1801105, rs7200210, rs4726107, and combinations thereof.

(h) The physiological response is a change in diastolic blood pressure and the plurality of SNP gene variants comprise at least one SNP gene variant selected from the group consisting of rs722341, rs3762272, rs600728, rs7556371, rs4531, rs2067477, rs660339, rs662, rs2162189, rs2702285, rs324651, and combinations thereof.

(i) The physiological response is a change in body mass and the plurality of SNP gene variants comprise at least one SNP gene variant selected from the group consisting of rs870995, rs600728, rs676643, rs2070424, rs1801278, rs6700734, rs4792887, rs6541017, rs1041163, rs722341, rs2162189, rs1255, rs1440451, rs3756007, and combinations thereof.

(j) The physiological response is a change in body mass index and the plurality of SNP gene variants comprise at least one SNP gene variant selected from the group consisting of rs5880, rs600728, rs676643, rs2070424, rs1801278, rs4792887, rs132642, rs2162189, rs1440451, rs936960, rs167771, and combinations thereof.

(k) The physiological response is a change in percentage fat and the plurality of SNP gene variants comprise at least one SNP gene variant selected from the group consisting of rs676643, rs2070424, rs885834, rs8178990, rs600728, rs8192708, rs6312, rs722341, rs1290443, and combinations thereof.

(l) The physiological response is a change in weight normalized maximum oxygen uptake and the plurality of SNP gene variants comprise at least one SNP gene variant selected from the group consisting of rs8178990, rs5447, rs1800871, rs4149056, rs7412, rs1901714, rs2298122, rs26312, rs563895, rs5896, rs3917550, rs2296189, rs1356413, and combinations thereof.

(m) The physiological response is a change in maximum oxygen uptake and the plurality of SNP gene variants comprise at least one SNP gene variant selected from the group consisting of rs11503016, rs2515449, rs334555, rs722341, rs4149056, rs7412, rs1396862, rs2515449, rs1805002, rs597316, rs26312, rs2020933, rs563895, rs5896, and combinations thereof.

One embodiment of the present invention involves obtaining nucleic acid, e.g. DNA, from a blood sample of a subject, and assaying the DNA to determine the individuals' genotype of one or a combination of the marker genes associated with physiological response to exercise. Other sampling procedures include but are not limited to buccal swabs, saliva, or hair root. In a preferred embodiment, genotyping is performed using a gene array methodology, which can be readily and reliably employed in the screening and evaluation methods according to this invention. A number of gene arrays are commercially available for use by the practitioner, including, but not limited to, static (e.g. photolithographically set), suspended beads (e.g. soluble arrays), and self assembling bead arrays (e.g. matrix ordered and deconvoluted). More specifically, the nucleic acid array analysis allows the establishment of a pattern of genetic variability from multiple genes and facilitates an understanding of the complex interactions that are elicited in an individual in response to exercise.

In a specific embodiment, the array consists of several hundred genes and is capable of genotyping hundreds of DNA polymorphisms simultaneously. Candidate genes for use in the arrays of the present invention are identified by various means including, but not limited to, pre-existing clinical databases and DNA repositories, review of the literature, and consultation with clinicians, differential gene expression models, physiological pathways in metabolism, cholesterol and lipid homeostasis, and from previously discovered genetic associations.

Another specific aspect of the method involves obtaining DNA from a subject, and assaying the genetic material to determine if any of the SNP gene variants belonging to the marker gene set are present, wherein the presence of the one or more SNP gene variants is predictive of physiological response to exercise. Micro- and nano-array analysis of the subject's DNA is preferred in this specific aspect of the invention.

In another aspect, the present invention provides methods for the identification of a population of individuals that will respond favorably to exercise based on the physiological responses of change in blood triglyceride level; blood LDL cholesterol level; blood HDL cholesterol level; ratio of total cholesterol to HDL cholesterol; LDL cholesterol, small fraction level; HDL cholesterol, large fraction level; blood glucose level; systolic blood pressure; diastolic blood pressure; body mass; body mass index; fat percentage; weight normalized maximum oxygen uptake; maximum oxygen uptake, or any combination of these responses. These individuals, who are identified through screening using the methods of the present invention, are especially likely to benefit from exercise.

In another aspect, the present invention further provides a method for the development of novel diagnostic systems, termed “physiotypes”, which are developed from combinations of gene polymorphisms and baseline characteristics, to provide practitioners with individualized patient response profiles for physiological response to exercise.

Yet another aspect of the present invention provides a system containing a support or support material, e.g. a micro- or nano-array, comprising a novel set of marker genes and/or gene variants associated with physiological response to exercise in a form suitable for the practitioner to employ in a screening assay for determining an individual's genotype. In addition to the marker genes and gene variants, the system comprises an algorithm for predicting the physiological response to exercise based on a predetermined set of mathematical equations providing specific coefficients to each of the components of the array.

The ensembles, arrays, methods, and systems of the invention are contemplated to be useful to practitioners as a tool to promote exercise compliance. Beyond the standard life modification advice of “exercise and be physically active”, the physician can now be precise and scientific in suggesting a fitness regimen and can provide additional motivational factors including improving cholesterol profiles prior to utilization of drugs, reducing body fat and lowering weight and having a general positive effect on several physiological outcomes. These capabilities point out the emergence of exercise as a medical fitness prescription. Further, there is contemplated to be utility in the management of metabolic syndrome and its individual components, dyslipidemias, obesity, diabetes, and hypertension. The possibility of a physiological treatment, as opposed to drugs, introduces an entire new dimension and scientific empowerment to “life style modification.” Conversely, for individuals where the exercise response tends more toward body weight and fat, exercise becomes a true complement to diet. Also, there are expected to be benefits in healthcare integration with the possibility of the doctor supporting the exercise prescription with a supervised fitness program or referring a patient to an exercise physiologist, physical therapist or fitness trainer.

### EXAMPLE 1

The recruitment of subjects, exercise training protocol, and physiological measurements used in this study are generally described in Thompson P D et al, Metabolism Vol. 53, No. 2, pp. 193-202 (2004), the contents of which is hereby incorporated by reference. Subjects were recruited at eight locations. Subjects initiated exercise training and completed a six month program. Subjects were recruited if they were: healthy and without orthopedic problems, non-smokers, physically inactive, ages from 18 to 70 years, and consumed two or fewer alcoholic beverages daily. Subjects were considered physically inactive if they participated in vigorous activity four or fewer times per month for the prior 6 months. Individuals were not recruited if their body mass index (BMI) exceeded 31, as caloric restriction reduces HDL-C. Subjects were avoided who might restrict their caloric intake during lipid measurement. Subjects underwent a medical history evaluation, physical exam, and a maximal exercise test to detect unreported abnormalities and occult coronary artery disease.

DNA was extracted from blood leukocytes for each subject. Genotyping was performed using the Illumina BeadArray™ platform and the GoldenGate™ assay (Oliphant et al, Biotechniques 32: S56-S61 (2002). For serum lipid and lipoprotein measurements, serum samples (preferably in duplicate) were obtained after a 12 hour fast before the start and after six months of exercise training. Post-training samples were obtained within 24 hours of the penultimate and final exercise training session. Lipid levels in women before and after training were obtained within ten days of the onset of menses to avoid variations in lipoprotein values (Culliname E M et al, Metabolism 44:565 (1995)). Serum was separated from plasma and frozen at −70 degrees Celsius until analyzed by the Lipid Research Laboratory, Lifespan Health System, Brown University, Providence (RI). All samples from an individual subject were analyzed in the same analysis run at the end of the study to minimize the effect of laboratory variation. Total cholesterol, TGs, LDL-C, HDL-C, and subfractions were determined using standard techniques (Thompson P D et al, Metabolism 46:217 (1997)).

For anthropometric measurements, body weight and height were measured using balance beam scales and wall mounted tape measures. Skinfold thickness was measured on the right side of the body using calipers to estimate percent body fat in men and women.

To determine maximal exercise capacity, subjects underwent two pre- and one post-training maximal treadmill exercise tests using the modified Astrand protocol (Pollack M L et al, Exercise in Health and Disease, Saunders, Philadelphia, Pa., 1984). The first pre-training test was designed to detect occult ischemia and to familiarize subjects with the measurement protocol, but was not used in data analysis. Blood pressure and 12-lead ECG, as well as expired oxygen, carbon dioxide, and ventilatory volume were measured. Maximal oxygen uptake was defined as the average of the two highest consecutive 30-second values at peak exercise.

Subjects were requested to maintain their usual dietary composition throughout the study. Dietary calories and composition were assessed by random, 24-hour dietary recalls. Trained dieticians called the subjects by telephone on one weekday and one weekend day before the start and during the last month of exercise training. Results from the two calls were averaged to estimate dietary intake.

Subjects underwent a progressive, supervised exercise training program. The duration of each exercise session was increased from 15 to 40 minutes during the first four weeks. Subjects exercised between 60 and 85% of their maximal exercise capacity based on their pre-determined maximal heart rate. Once subjects could perform 40 minutes of exercise, they continued this duration of exercise 4 days a week for an additional 5 months for a total of 6 months of participation. Subjects also participated in 5 minutes of warm-up and cool-down so that each workout required 50 minutes. Treadmill exercise was the primary mode of training but subjects were able to use a variety of training modalities including treadmills, stationary cycles, cross-country ski machines, stair steppers, and rowing machines for variety and to minimize orthopedic injury.

Weekly exercise energy expenditure expressed as kilocalories per week was estimated from the average heart rates recorded for exercise sessions of that week. From individual plots of VO₂ vs. heart rate created from pre-training maximal exercise test data, we estimated the VO₂ corresponding to the training exercise heart rate intensity and multiplied that VO₂ by training session duration to obtain total oxygen consumption for each bout. Each liter of oxygen was assumed to represent 5 kilocalories of energy expenditure.

We tested the inventive method by examining the effects of exercise on blood triglyceride level (log transformed); blood LDL cholesterol level; blood HDL cholesterol level; ratio of total cholesterol to HDL cholesterol; LDL cholesterol, small fraction level; HDL cholesterol, large fraction level; blood glucose level; systolic blood pressure; diastolic blood pressure; body mass; body mass index; fat percentage; weight normalized maximum oxygen uptake; and maximum oxygen uptake, as a function of various SNP markers. We correlated the exercise responses as measured by various outcomes with the variability of selected candidate genes using physiogenomics. Physiogenomics was used as a technique to explore the variability in patient response to exercise. Physiogenomics is a medical application of sensitivity analysis [Ruaño, et al., Physiogenomics: Integrating systems engineering and nanotechnology for personalized health. In: Joseph. D. Bronzino, ed. The Biomedical Engineering Handbook, 3rd edition, 2006.]. Sensitivity analysis is the study of the relationship between the input and the output of a model and the analysis, utilizing systems theory, of how variation of the input leads to changes in output quantities. Physiogenomics utilizes as input the variability in genes, measured by single nucleotide polymorphisms (SNP) and determines how the SNP frequency among individuals relates to the variability in physiological characteristics, the output.

The goal of the investigation was to develop physiogenomic markers for predicting physiological response to exercise by using an informatics platform to analyze data from exercise studies.

Potential associations of marker genes to exercise. Various SNPs associated with, for example, the observation of lipid level and BMI changes in patients undergoing exercise treatment were screened. The endpoints analyzed were log of blood triglyceride level; blood LDL cholesterol level; blood HDL cholesterol level; ratio of total cholesterol to HDL cholesterol; LDL cholesterol, small fraction level; HDL cholesterol, large fraction level; blood glucose level; systolic blood pressure; diastolic blood pressure; body mass; body mass index; fat percentage; weight normalized maximum oxygen uptake; and maximum oxygen uptake. The physiogenomic model was developed using the following procedure: 1) Establish a baseline model using only the demographic and clinical variables, 2) Screen for associated genetic markers by testing each SNP against the unexplained residual of the baseline model, and 3) Establish a revised model incorporating the significant associations from the SNP screen. All models are simple linear regression models, but other well-known statistical methods are contemplated to be useful.

Tables 6-19 list the SNPs that have been found to be associated with each outcome with only SNPs with a statistical significance level of 0.05 being shown. The baseline variables (covariates) broken down by demographic factors are shown in Tables 20 and 21, where the variables indicated as “pre” represent the initial value of the indicated response.

TABLE 6
SNPs with statistical significance level of 0.05 for change in LDL

| var | snp | gene | pval | adj | mpv | mr2 | degf | coeff | apv | ar2 | SNP type | Gene Name |
|---|---|---|---|---|---|---|---|---|---|---|---|---|
| ldl.chg | Total | 0 | 0.381 | 0.38 | 3E−10 | 56.1% | 71 | 0.51 | 1E−09 | 48.6% | | |
| ldl.chg | rs2005590 | APOL4 | 5E−04 | 0.13 | 3E−05 | 12.2% | 89 | 10.12 | 2E−05 | 10.6% | ~1 kb upstream | apolipoprotien L, 4 |
| ldl.chg | rs3118536 | RXRA | 0.004 | 0.68 | NA | NA | 92 | 10.68 | 2E−04 | 7.6% | intron 3 | retinoid X receptor, alpha |
| ldl.chg | rs1041163 | VCAM1 | 0.008 | 0.88 | 0.02 | 3.5% | 87 | 8.858 | 0.001 | 5.6% | ~150 bp upstream | vascular cell adhesion |
| | | | | | | | | | | | | molecule 1 |
| ldl.chg | rs334555 | GSK3B | 0.009 | 0.92 | NA | NA | 89 | −8.41 | 0.013 | 3.3% | intron 1 | glycogen synthase |
| | | | | | | | | | | | | kinase 3 beta |
| ldl.chg | rs6960931 | PRKAG2 | 0.012 | 0.96 | NA | NA | 88 | −11.4 | 0.092 | 1.5% | intron 1 | protein kinase, |
| | | | | | | | | | | | | AMP-activated, |
| | | | | | | | | | | | | gamma 2 non- |
| | | | | | | | | | | | | catalytic subunit |
| ldl.chg | rs1800471 | TGFB1 | 0.012 | 0.96 | 2E−04 | 9.9% | 92 | 12.52 | 0.043 | 2.2% | exon 1, R25P | transforming growth |
| | | | | | | | | | | | | factor, beta 1 |
| | | | | | | | | | | | | (Camurati-Engelmann |
| | | | | | | | | | | | | disease) |
| ldl.chg | rs1799978 | DRD2 | 0.012 | 0.97 | 5E−06 | 15.0% | 92 | −13.1 | 4E−04 | 7.0% | ~500 bp upstream | dopmine receptor D2 |
| ldl.chg | rs707922 | APOM | 0.015 | 0.99 | 0.063 | 2.2% | 90 | 11.52 | 0.012 | 3.4% | intron 5 | apolipoprotein M |
| ldl.chg | rs870995 | PIK3CA | 0.032 | 1.00 | 2E−04 | 9.3% | 88 | −5.35 | 0.007 | 3.9% | ~3.3 kb upstream | phosphoinositide-3- |
| | | | | | | | | | | | | kinase, catalytic, |
| | | | | | | | | | | | | alpha polypeptide |
| ldl.chg | rs2162189 | SST | 0.042 | 1.00 | NA | NA | 92 | 10.17 | 0.724 | 0.1% | ~2.5 kbp | somatostatin |
| | | | | | | | | | | | | upstream |
| ldl.chg | rs5092 | APOA4 | 0.043 | 1.00 | 0.043 | 2.6% | 89 | −6.75 | 0.286 | 0.6% | exon 2, T29T | apolipoprotein A-IV |
| ldl.chg | rs1398176 | GABRA4 | 0.046 | 1.00 | 0.152 | 1.3% | 84 | −7.73 | 0.113 | 1.3% | intron 8 | gamma-aminobutyric |
| | | | | | | | | | | | | acid (GABA) A |
| | | | | | | | | | | | | receptor, alpha 4 |
| ldl.chg | rs2069827 | IL6 | 0.047 | 1.00 | NA | NA | 91 | −8.63 | 0.08 | 1.6% | ~1.5 kb upstream | interleukin 6 |
| | | | | | | | | | | | | (interferon, beta 2) |

TABLE 7
SNPs with statistical significance level of 0.05 for change in HDL

| var | snp | gene | pval | adj | mpv | mr2 | degf | coeff | apv | ar2 | SNP type | Gene Name |
|---|---|---|---|---|---|---|---|---|---|---|---|---|
| hdl.chg | Total | 0 | 0.316 | 0.32 | 1E−04 | 31.5% | 71 | −0.35 | 3E−06 | 35.7% | | |
| hdl.chg | rs376096 | CCL2 | 0.003 | 0.62 | 0.035 | 4.5% | 90 | 2.655 | 7E−04 | 7.8% | ~500 bp upstream | chemokine (C—C motif) |
| | | | | | | | | | | | | ligand 2 |
| hdl.chg | rs3791981 | APOB | 0.009 | 0.93 | NA | NA | 92 | −3.04 | 0.007 | 4.8% | intron 18 | apolipoprotein B (including |
| | | | | | | | | | | | | Ag(x) antigen) |
| hdl.chg | rs1143634 | IL1B | 0.011 | 0.95 | 0.007 | 7.6% | 91 | −2.31 | 0.011 | 4.3% | exon 4, F105F | interleukin 1, beta |
| hdl.chg | rs10513055 | PIK3CB | 0.023 | 1.00 | 0.038 | 4.3% | 92 | 2.072 | 0.007 | 4.8% | intron 6 | phosphoinositide-3-kinase, |
| | | | | | | | | | | | | catalytic, beta polypeptide |
| hdl.chg | rs916829 | ABCC8 | 0.027 | 1.00 | NA | NA | 92 | −2.71 | 0.271 | 0.8% | intron 16 | ATP-binding cassette, |
| | | | | | | | | | | | | sub-family C (CFTR/MRP), |
| | | | | | | | | | | | | member 8 |
| hdl.chg | rs894251 | SCARB2 | 0.028 | 1.00 | NA | NA | 92 | 2.512 | 0.191 | 1.1% | intron 1 | scavenger receptor |
| | | | | | | | | | | | | class B, member 2 |
| hdl.chg | rs1891311 | HTR7 | 0.029 | 1.00 | 0.135 | 2.2% | 88 | −4.04 | 0.044 | 2.7% | ~700 bp upstream | 5-hydroxytryptamine |
| | | | | | | | | | | | | (serotonin) receptor 7 |
| | | | | | | | | | | | | (adenylate cyclase-coupled) |
| hdl.chg | rs1800871 | IL10 | 0.032 | 1.00 | 0.01 | 6.8% | 93 | 2.138 | 0.004 | 5.7% | ~700 bp upstream | interleukin 10 |
| hdl.chg | rs521674 | ADRA2A | 0.04 | 1.00 | NA | NA | 82 | −1.75 | 0.094 | 1.8% | ~1.5 kb upstream | adrenergic, alpha-2A-, |
| | | | | | | | | | | | | receptor |
| hdl.chg | rs5883 | CETP | 0.045 | 1.00 | NA | NA | 91 | 3.338 | 0.565 | 0.2% | exon 9, F287F | cholesteryl ester transfer |
| | | | | | | | | | | | | protein, plasma |
| hdl.chg | rs5049 | AGT | 0.046 | 1.00 | 0.014 | 6.1% | 84 | −2.92 | 0.09 | 1.9% | ~150 bp upstream | angiotensinogen (serine |
| | | | | | | | | | | | | (or cysteine) proteinase |
| | | | | | | | | | | | | inhibitor, clade A |
| | | | | | | | | | | | | (alpha-1 antiproteinase, |
| | | | | | | | | | | | | antitrypsin), member 8) |

TABLE 8
SNPs with statistical significance level of 0.05 for change in log(tg)

| var | snp | gene | pval | adj | mpv | mr2 | degf | coeff | apv | ar2 | SNP type | Gene Name |
|---|---|---|---|---|---|---|---|---|---|---|---|---|
| logtg.chg | Total | 0 | 0.76 | 0.76 | 3E−07 | 58.5% | 55 | −0.09 | 6E−06 | 41.9% | | |
| logtg.chg | rs26312 | GHRL | 0.006 | 0.80 | NA | NA | 93 | 0.153 | 0.002 | 5.7% | ~1 kb upstream | ghrelin precursor |
| logtg.chg | rs7602 | LEPR | 0.009 | 0.92 | NA | NA | 97 | −0.12 | 0.012 | 3.8% | intron 1 (3′ | leptin receptor |
| | | | | | | | | | | | UTR on | |
| | | | | | | | | | | | another gene) | |
| logtg.chg | rs11503016 | GABRA2 | 0.011 | 0.96 | 0.053 | 2.9% | 94 | 0.123 | 0.011 | 3.9% | intron 3 | gamma-aminobutyric acid |
| | | | | | | | | | | | | (GABA) A receptor alpha 2 |
| logtg.chg | rs4890109 | RARA | 0.011 | 0.96 | NA | NA | 95 | −0.18 | 0.07 | 2.0% | intron 3 | retinoic acid receptor, alpha |
| logtg.chg | rs2070586 | DAO | 0.014 | 0.98 | 0.008 | 5.7% | 97 | 0.127 | 0.007 | 4.4% | intron 1 | D-amino-acid oxidase |
| | | | | | | | | | | | (untranslated?) | |
| logtg.chg | CETP | NA | 0.015 | 0.98 | 0.002 | 8.1% | 75 | 0.092 | 0.037 | 2.6% | | |
| logtg.chg | rs2278718 | MDH1 | 0.015 | 0.99 | 0.172 | 1.4% | 96 | −0.11 | 0.17 | 1.1% | ~550 bp | malate dehydrogenase 1, |
| | | | | | | | | | | | upstream | NAD (soluble) |
| logtg.chg | rs908867 | BDNF | 0.018 | 0.99 | 7E−04 | 9.7% | 94 | −0.14 | 0.023 | 3.1% | ~2 kb upstream | brain-derived |
| | | | | | | | | | | | | neurotrophic factor |
| logtg.chg | rs4121817 | PIK3C3 | 0.02 | 1.00 | 0.021 | 4.3% | 96 | −0.14 | 0.103 | 1.6% | intro 10 | phosphoinositide.-3- |
| | | | | | | | | | | | | kinase, class 3 |
| logtg.chg | rs2240403 | CRHR2 | 0.02 | 1.00 | 0.006 | 6.1% | 93 | −0.16 | 0.009 | 4.2% | exon 10, S349S | corticotropin releasing |
| | | | | | | | | | | | | hormone receptor 2 |
| logtg.chg | rs722341 | ABCC8 | 0.021 | 1.00 | NA | NA | 95 | −0.13 | 0.179 | 1.1% | intron 7 | ATP-binding cassette, |
| | | | | | | | | | | | | sub-family C (CFTR/MRP), |
| | | | | | | | | | | | | member 8 |
| logtg.chg | rs4795180 | ACACA | 0.027 | 1.00 | NA | NA | 95 | 0.113 | 0.168 | 1.1% | intron 31 | acetyl-Coenzyme A |
| | | | | | | | | | | | | carboxylase alpha |
| logtg.chg | rs2276307 | HTR3B | 0.037 | 1.00 | 0.015 | 4.8% | 94 | 0.087 | 0.099 | 1.6% | intron 6 | 5-hydroxytryptamine |
| | | | | | | | | | | | | (serotonin) receptor 3B |
| logtg.chg | rs916829 | ABCC8 | 0.04 | 1.00 | NA | NA | 97 | 0.118 | 0.133 | 1.3% | intron 16 | ATP-binding cassette, |
| | | | | | | | | | | | | sub-family C (CFTR/MRP), |
| | | | | | | | | | | | | member 8 |
| logtg.chg | rs2162189 | SST | 0.043 | 1.00 | NA | NA | 97 | 0.141 | 0.201 | 1.0% | ~2.5 kbp | somatostatin |
| | | | | | | | | | | | upstream | |
| logtg.chg | rs563895 | AVEN | 0.045 | 1.00 | 0.02 | 4.3% | 98 | 0.106 | 0.161 | 1.2% | intron 2 | apoptosis, caspase |
| | | | | | | | | | | | | activation inhibitor |
| logtg.chg | rs1800871 | IL10 | 0.046 | 1.00 | NA | NA | 97 | 0.092 | 0.384 | 0.4% | ~700 bp | interleukin 10 |
| | | | | | | | | | | | upstream | |
| logtg.chg | rs1171276 | LEPR | 0.047 | 1.00 | 0.003 | 7.5% | 87 | −0.09 | 0.239 | 0.8% | intron 1 | leptin receptor |
| | | | | | | | | | | | (untranslated) | |
| logtg.chg | rs10460960 | CCK | 0.049 | 1.00 | 0.031 | 3.7% | 96 | 0.101 | 0.175 | 1.1% | ~2.5 kb | cholecystokinin |
| | | | | | | | | | | | upstream | |

TABLE 9
SNPs with statistical significance level of 0.05 for change in blood glucose (glu)

| var | snp | gene | pval | adj | mpv | mr2 | degf | coeff | apv | ar2 | SNP type | Gene Name |
|---|---|---|---|---|---|---|---|---|---|---|---|---|
| glu.chg | Total | 0 | 0.615 | 0.61 | 1E−08 | 55.4% | 67 | 4.412 | 6E−08 | 49.6% | | |
| glu.chg | rs322695 | RARB | 0.002 | 0.35 | 7E−05 | 12.0% | 85 | −3.58 | 1E−04 | 9.2% | ~100 kb | retinoic acid receptor, beta |
| | | | | | | | | | | | upstream | |
| glu.chg | rs3822222 | CCKAR | 0.006 | 0.81 | 0.001 | 7.5% | 86 | 3.25 | 0.015 | 3.5% | intron 2 | cholecystokinin A receptor |
| glu.chg | rs5361 | SELE | 0.013 | 0.98 | 0.019 | 3.8% | 85 | −1.91 | 0.011 | 3.8% | exon 3, R149S | seleotin E (endothelial |
| | | | | | | | | | | | | adhesion molecule 1) |
| glu.chg | rs737865 | TXNRD2 | 0.017 | 0.99 | NA | NA | 83 | −1.92 | 0.067 | 2.0% | ~800 bp | thioredoxin reductase 2 |
| | | | | | | | | | | | upstream in | |
| | | | | | | | | | | | intron 1 of | |
| | | | | | | | | | | | COMT | |
| glu.chg | rs6131 | SELP | 0.018 | 0.99 | NA | NA | 85 | −2.49 | 0.011 | 3.9% | exon 7, N331S | selectin P (granule membrane |
| | | | | | | | | | | | | protein 140 kDa, |
| | | | | | | | | | | | | antigen CD62) |
| glu.chg | rs722341 | ABCC8 | 0.021 | 1.00 | 4E−04 | 9.1% | 85 | 2.751 | 5E−04 | 7.4% | intron 7 | ATP-binding cassette, |
| | | | | | | | | | | | | sub-family C (CFTR/MRP), |
| | | | | | | | | | | | | member 8 |
| glu.chg | rs10508244 | PFKP | 0.032 | 1.00 | 0.031 | 3.2% | 84 | −3.04 | 0.041 | 24% | intron 10 | phosphofructokinase, platelet |
| glu.chg | rs1042718 | ADRB2 | 0.032 | 1.00 | 0.033 | 3.2% | 83 | −2.2 | 0.044 | 2.4% | exon 1, R175R | adrenergic, beta-2-, |
| | | | | | | | | | | | | receptor, surface |
| glu.chg | rs2229126 | ADRA1A | 0.035 | 1.00 | 0.019 | 3.9% | 86 | 6.455 | 0.048 | 2.3% | intron 1, | adrenergic, alpha-1A-,receptor |
| | | | | | | | | | | | alternative | |
| | | | | | | | | | | | transcript: | |
| | | | | | | | | | | | D465E, exon 1 | |
| glu.chg | rs1800808 | SELP | 0.036 | 1.00 | NA | NA | 82 | −2.63 | 0.468 | 0.3% | ~250 bp | selectin P (granule membrane |
| | | | | | | | | | | | upstream | protein 140 kDa, |
| | | | | | | | | | | | | antigen CD62) |
| glu.chg | rs107540 | CRHR2 | 0.04 | 1.00 | 8E−04 | 8.2% | 86 | −1.61 | 0.018 | 3.3% | ~18 kb | Corticotropin-releasing |
| | | | | | | | | | | | upstream | hormone receptor 2 |
| glu.chg | rs1322783 | DISC1 | 0.043 | 1.00 | 0.055 | 2.5% | 87 | −2.33 | 0.002 | 5.7% | intron 6 | disrupted in schizophrenia 1 |
| glu.chg | rs4531 | DBH | 0.044 | 1.00 | NA | NA | 86 | 2.605 | 0.712 | 0.1% | exon 5, S304A | dopamine beta-hydroxylase |
| | | | | | | | | | | | | (dopamine beta- |
| | | | | | | | | | | | | monooxygenase) |
| glu.chg | rs2070424 | SOD1 | 0.045 | 1.00 | 0.087 | 2.0% | 85 | −3.7 | 0.019 | 3.2% | intron 3 | superoxide dismutase 1, |
| | | | | | | | | | | | | soluble (amyotrophic |
| | | | | | | | | | | | | lateral sclerosis 1 (adult)) |
| glu.chg | rs10082776 | RARG | 0.045 | 1.00 | NA | NA | 85 | −2.58 | 0.549 | 0.2% | intron 2 | retinoic acid receptor, gamma |
| | | | | | | | | | | | (untranslated) | |
| glu.chg | rs2702285 | AVEN | 0.048 | 1.00 | NA | NA | 84 | −1.7 | 0.897 | 0.0% | intron 1 (MT) | apoptosis, caspase activation |
| | | | | | | | | | | | | inhibitor |

TABLE 10
SNPs with statistical significance level of 0.05 for change in LDL, small fraction (ldlsm)

| var | snp | gene | pval | adj | mpv | mr2 | degf | coeff | apv | ar2 | SNP type | Gene Name |
|---|---|---|---|---|---|---|---|---|---|---|---|---|
| ldlsm.chg | Total | 0 | 0.036 | 0.04 | 3E−09 | 62.9% | 59 | −13.7 | 5E−06 | 45.8% | | |
| ldlsm.chg | rs2076672 | APOL5 | 0.004 | 0.66 | 0.011 | 4.3% | 83 | 11.47 | 0.002 | 6.4% | exon 3, M323T | apolipoprotein L, 5 |
| ldlsm.chg | rs5880 | CETP | 0.006 | 0.84 | NA | NA | 85 | 16.43 | 0.013 | 4.0% | nonsynonymous, | cholesteryl ester |
| | | | | | | | | | | | P390A | transfer protein, |
| | | | | | | | | | | | | plasma |
| ldlsm.chg | rs1150226 | HTR3A | 0.007 | 0.84 | NA | NA | 87 | −25.1 | 0.013 | 4.0% | ~500 bp upstream | 5- |
| | | | | | | | | | | | | hydroxytryptamine |
| | | | | | | | | | | | | (serotonin) |
| | | | | | | | | | | | | receptor 3A |
| ldlsm.chg | rs6131 | SELP | 0.012 | 0.9 | 1E−06 | 18.8% | 85 | 12.61 | 0.003 | 5.9% | exon 7, N331S | selectin p (granule |
| | | | | | | | | | | | | membrane protein |
| | | | | | | | | | | | | 140 kDa, |
| | | | | | | | | | | | | antigen CD62) |
| ldlsm.chg | rs4917348 | RXRA | 0.013 | 0.98 | 0.112 | 1.6% | 75 | −13.3 | 0.042 | 2.7% | ~100 kbp | retinoid X receptor, |
| | | | | | | | | | | | upstream | alpha |
| ldlsm.chg | rs8192708 | PCK1 | 0.013 | 0.98 | NA | NA | 83 | 15.22 | 0.016 | 3.7% | exon 5, V2671 | phospho- |
| | | | | | | | | | | | | enolpyruvate |
| | | | | | | | | | | | | carboxykniase 1 |
| | | | | | | | | | | | | (soluble) |
| ldlsm.chg | rs885834 | CHAT | 0.017 | 0.99 | 0.197 | 1.1% | 85 | 8.012 | 0.029 | 3.1% | ~450 bp upstream | choline |
| | | | | | | | | | | | | acetyltransferase |
| ldlsm.chg | rs4675096 | IRSI | 0.017 | 0.99 | 0.071 | 2.1% | 86 | −12.8 | 0.188 | 1.1% | ~4 kb upstream | insulin receptor |
| | | | | | | | | | | | | substrate-1 |
| ldlsm.chg | rs1131010 | PECAM1 | 0.019 | 1.00 | 2E−06 | 17.1% | 83 | −21.9 | 0.311 | 0.6% | intron 10 | platelet/endothelial |
| | | | | | | | | | | | | cell adhesion |
| | | | | | | | | | | | | molecule (CD31 |
| | | | | | | | | | | | | antigen) |
| ldlsm.chg | rs6092 | SERPINE1 | 0.02 | 1.00 | 0.159 | 1.3% | 85 | 14.12 | 0.078 | 2.0% | exon 1, T15A | serine (or cysteine) |
| | | | | | | | | | | | | proteinase inhibitor, |
| | | | | | | | | | | | | clade E (nexin, |
| | | | | | | | | | | | | plasminogen |
| | | | | | | | | | | | | activator |
| | | | | | | | | | | | | inhibitor type 1) |
| | | | | | | | | | | | | member 1 |
| ldlsm.chg | rs10515070 | PIK3R1 | 0.021 | 1.00 | NA | NA | 78 | −9.29 | 0.011 | 4.2% | intron 1 | phosphoinositide-3- |
| | | | | | | | | | | | | kinase, regulatory |
| | | | | | | | | | | | | subunite 1 |
| | | | | | | | | | | | | (p85 alpha) |
| ldlsm.chg | rs6078 | LIPC | 0.029 | 1.00 | 0.064 | 2.2% | 87 | 17.29 | 0.424 | 0.4% | exon3, M95V | lipase. hepatic |
| ldlsm.chg | rs1805002 | CCKBR | 0.03 | 1.00 | NA | NA | 86 | 17.7 | 0.086 | 1.9% | I125V, exon2 | cholecystokinin |
| | | | | | | | | | | | | B receptor |
| ldlsm.chg | rs10890819 | ACAT1 | 0.031 | 1.00 | 0.013 | 4.1% | 85 | 8.09 | 0.171 | 1.2% | intron 10 | acetyl-Coenzyme A |
| | | | | | | | | | | | | acetyltransferase |
| | | | | | | | | | | | | 1 (acetoacetyl |
| | | | | | | | | | | | | Coenzyme A |
| | | | | | | | | | | | | thiolase) |
| ldlsm.chg | rs659734 | HTR2A | 0.04 | 1.00 | 0.016 | 3.9% | 85 | 15.25 | 0.082 | 1.9% | intron | 5-hydroxy- |
| | | | | | | | | | | | | tryptamine |
| | | | | | | | | | | | | (serotonin) |
| | | | | | | | | | | | | receptor 2A |
| ldlsm.chg | rs83060 | VEGF | 0.041 | 1.00 | NA | NA | 81 | 7.698 | 0.441 | 0.4% | ~2.5 kb upstream | vascular endothelial |
| | | | | | | | | | | | | growth factor |
| ldlsm.chg | rs706713 | PIK3R1 | 0.045 | 1.00 | 0.003 | 6.3% | 83 | −8.3 | 0.45 | 0.4% | exon 1, Y73Y | phosphoinositide- |
| | | | | | | | | | | | | 3-kinase, |
| | | | | | | | | | | | | regulatory subunit 1 |
| | | | | | | | | | | | | (p85 alpha) |
| ldlsm.chg | rs2032582 | ABCB1 | 0.048 | 1.00 | NA | NA | 81 | 7.557 | 0.079 | 2.0% | exon 20, | ATP-binding |
| | | | | | | | | | | | TPAS 893 | cessette, sub-family |
| | | | | | | | | | | | | B (MDR/TAP), |
| | | | | | | | | | | | | member 1 |

TABLE 11
SNPs with statistical significance level of 0.05 for change in HDL, large fraction (hdllg)

| var | snp | gene | pval | adj | mpv | mr2 | degf | coeff | apv | ar2 | SNP type | Gene Name |
|---|---|---|---|---|---|---|---|---|---|---|---|---|
| hdllg.chg | Total | 0 | 0.96 | 0.96 | 6E−05 | 36.6% | 62 | −1.99 | 8E−06 | 36.5% | | |
| hdllg.chg | rs1800871 | IL10 | 0.001 | 0.34 | 0.001 | 11.4% | 81 | 5.856 | 2E−04 | 11.0% | ~700 bp upstream | interleukin 10 |
| hdllg.chg | rs10513055 | PIK3CB | 0.01 | 0.93 | 0.008 | 7.8% | 80 | 4.176 | 0.001 | 8.2% | intron 6 | phosphoinositide-3- |
| | | | | | | | | | | | | kinase, catalytic, |
| | | | | | | | | | | | | beta polypeptide |
| hdllg.chg | APOA1 | NA | 0.012 | 0.97 | NA | NA | 71 | −4.4 | 0.027 | 3.6% | | |
| hdllg.chg | rs4520 | APOC3 | 0.015 | 0.99 | 0.048 | 4.2% | 79 | −4.08 | 0.122 | 1.8% | G34G | apolipoprotein C-III |
| hdllg.chg | rs1042718 | ADRB2 | 0.016 | 0.99 | 0.055 | 3.9% | 79 | −3.95 | 0.013 | 4.6% | exon 1, R175R | adrenergic, beta-2-, |
| | | | | | | | | | | | | receptor, surface |
| hdllg.chg | rs5049 | AGT | 0.019 | 1.00 | 0.014 | 6.5% | 73 | −6.18 | 0.103 | 2.0% | ~150 bp upstream | angiotensinogen (serine |
| | | | | | | | | | | | | (or cysteine) proteinase |
| | | | | | | | | | | | | inhibitor, clade A |
| | | | | | | | | | | | | (alpha-1 antiproteinase, |
| | | | | | | | | | | | | antitrypsin), member 8) |
| hdllg.chg | rs3760396 | CCL2 | 0.026 | 1.00 | 0.105 | 2.8% | 79 | 3.487 | 0.055 | 2.7% | ~500 bp upstream | chemokine (C—C motif) |
| | | | | | | | | | | | | ligand 2 |
| hdllg.chg | rs2020933 | SLC6A4 | 0.031 | 1.00 | NA | NA | 80 | 6.664 | 0.131 | 1.7% | intron 1 | solute carrier family 6 |
| | | | | | | | | | | | | (neurotransmitter |
| | | | | | | | | | | | | transporter, |
| | | | | | | | | | | | | serotonin), member 4 |
| hdllg.chg | rs6586179 | LIPA | 0.038 | 1.00 | NA | NA | 80 | 5.173 | 0.395 | 0.5% | exon 1, R23G | lipase A lysosomal acid, |
| | | | | | | | | | | | | cholesterol esterase |
| | | | | | | | | | | | | (Wolman disease) |
| hdllg.chg | rs3822222 | CCKAR | 0.047 | 1.00 | NA | NA | 80 | 4.245 | 0.492 | 0.3% | intron 2 | cholecystokinin A |
| | | | | | | | | | | | | receptor |

TABLE 12
SNPs with statistical significance level of 0.05 for change in systolic blood pressure (sbp)

| var | snp | gene | pval | adj | mpv | mr2 | degf | coeff | apv | ar2 | SNP type | Gene Name |
|---|---|---|---|---|---|---|---|---|---|---|---|---|
| sbp.chg | Total | 0 | 0.865 | 0.86 | 7E−08 | 46.6% | 75 | 0.518 | 2E−06 | 38.3% | | |
| sbp.chg | rs1801105 | HNMT | 0.011 | 0.95 | 1E−04 | 11.7% | 96 | 5.936 | 0.003 | 5.5% | exon 4, I105T | histamine |
| | | | | | | | | | | | | N-methyltransferase |
| sbg.chg | rs597316 | CPT1A | 0.016 | 0.99 | NA | NA | 95 | −3.27 | 0.015 | 3.6% | ~28 kb upstream | carnitine |
| | | | | | | | | | | | | palmitoyltransferase 1A |
| sbp.chg | rs4149056 | SLCO1B1 | 0.017 | 0.99 | 0.068 | 2.4% | 93 | −4 | 0.013 | 3.8% | exon 5, A174V | solute carrier organic |
| | | | | | | | | | | | | anion transporter family, |
| | | | | | | | | | | | | member 1B1 |
| sbp.chg | rs697107 | WBSCR14 | 0.018 | 0.99 | 0.048 | 2.9% | 96 | −4.57 | 0.03 | 2.9% | intron 6 | Williams Beuren |
| | | | | | | | | | | | | syndrome chromosome |
| | | | | | | | | | | | | region 14 |
| sbp.chg | rs7200210 | SLC12A4 | 0.02 | 1.00 | 6E−04 | 9.2% | 97 | 6.155 | 0.008 | 4.3% | intron 14 | solute carrier family |
| | | | | | | | | | | | | 12 (potassium/chloride |
| | | | | | | | | | | | | transporters), member 4 |
| sbp.chg | rs10515070 | PIK3R1 | 0.023 | 1.00 | 0.001 | 7.9% | 88 | −2.98 | 0.041 | 2.5% | intron 1 | phosphoinositide-3- |
| | | | | | | | | | | | | kinase, regulalory |
| | | | | | | | | | | | | subunit 1 (p85 alpha) |
| sbp.chg | rs706713 | PIK3R1 | 0.032 | 1.00 | NA | NA | 93 | −2.94 | 0.934 | 0.0% | exon 1, Y73Y | phosphoinositide-3- |
| | | | | | | | | | | | | kinase, regulatory |
| | | | | | | | | | | | | subunit 1 (p85 alpha) |
| sbp.chg | rs1800871 | IL10 | 0.032 | 1.00 | 0.07 | 2.4% | 96 | 3.505 | 0.001 | 6.5% | ~700 bp upstream | interleukin 10 |
| sbp.chg | rs4726107 | PRKAG2 | 0.034 | 1.00 | 0.002 | 7.3% | 95 | 4.793 | 0.186 | 1.1% | ~2 kb upstream | protein kinase, AMP- |
| | | | | | | | | | | | | activated, gamma 2 non- |
| | | | | | | | | | | | | catalytic |
| sbp.chg | rs2298122 | DRD1IP | 0.035 | 1.00 | 0.056 | 2.7% | 92 | −3.42 | 0.004 | 5.1% | intron 1 | dopamine receptor D1 |
| | | | | | | | | | | | | interacting protein |
| sbp.chg | rs5896 | F2 | 0.039 | 1.00 | NA | NA | 91 | −3.72 | 0.232 | 0.9% | exon 6, M165T | coagulation factor |
| | | | | | | | | | | | | II (thrombin) |
| sbp.chg | rs207024 | SOD1 | 0.041 | 1.00 | NA | NA | 94 | 6.259 | 0.46 | 0.3% | intron 3 | superoxide dismutase 1, |
| | | | | | | | | | | | | soluble (amyotrophic |
| | | | | | | | | | | | | lateral sclerosis 1 (adult)) |
| sbp.chg | rs8178990 | CHAT | 0.042 | 1.00 | NA | NA | 96 | 5.506 | 0.09 | 1.7% | exon 4, F125L | choline acetyltransferase |
| | | | | | | | | | | | (MT) | |
| sbp.chg | rs1805002 | CCKBR | 0.05 | 1.00 | NA | NA | 96 | 5.851 | 0.745 | 0.1% | I125V, exon 2 | cholecystokinin B |
| | | | | | | | | | | | | receptor |

TABLE 13
SNPs with statistical significance level of 0.05 for change in diastolic blood pressure (dbp)

| var | snp | gene | pval | adj | mpv | mr2 | degf | coeff | apv | ar2 | SNP type | Gene Name |
|---|---|---|---|---|---|---|---|---|---|---|---|---|
| dbp.chg | Total | 0 | 0.186 | 0.19 | 2E−06 | 46.4% | 61 | 0.524 | 1E−05 | 39.7% | | |
| dbp.chg | rs3762272 | PKLR | 0.002 | 0.45 | NA | NA | 83 | −5.47 | 8E−04 | 8.0% | intron 2 | pyruvate kinase, liver and RBC |
| dbp.chg | rs722341 | ABCC8 | 0.003 | 0.52 | 3E−04 | 13.3% | 84 | −3.96 | 0.007 | 5.0% | intron 7 | ATP-binding cassette, |
| | | | | | | | | | | | | sub-family C (CFTR/MRP), |
| | | | | | | | | | | | | member 8 |
| dbp.chg | rs1556478 | LIPA | 0.011 | 0.95 | NA | NA | 83 | −2.48 | 0.072 | 2.2% | intron 5 | lipase A, lysosomal acid, |
| | | | | | | | | | | | | cholesterol esterase |
| | | | | | | | | | | | | (Wolman disease) |
| dbp.chg | rs2067477 | CHRM1 | 0.015 | 0.99 | 0.169 | 1.7% | 85 | −2.86 | 0.193 | 1.2% | exon 1, G89G | cholinergic receptor, |
| | | | | | | | | | | | | muscarinic 1 |
| dbp.chg | rs4531 | DBH | 0.017 | 0.99 | 0.022 | 4.8% | 84 | −3.41 | 0.021 | 3.7% | exon 5, S304A | dopamine beta- |
| | | | | | | | | | | | | hydroxylase(dopamine beta- |
| | | | | | | | | | | | | monooxygenase) |
| dbp.chg | rs7556371 | PIK3C2B | 0.028 | 1.00 | 0.001 | 10.4% | 83 | 2.097 | 0.019 | 3.8% | intron 1 | phosphoinositide-3-kinase, |
| | | | | | | | | | | | (untranslated?) | class 2, beta polypeptide |
| dbp.chg | rs2702285 | AVEN | 0.028 | 1.00 | NA | NA | 83 | 2.112 | 0.11 | 1.7% | intron 1 (MT) | apoptosis, caspase activation |
| | | | | | | | | | | | | inhibitor |
| dbp.chg | rs1438732 | NR3C1 | 0.031 | 1.00 | NA | NA | 82 | 2.513 | 0.229 | 1.0% | intron 1 | nuclear receptor subfamily 3, |
| | | | | | | | | | | | | group C, member 1 |
| | | | | | | | | | | | | (glucocorticoid receptor) |
| dbp.chg | rs2228502 | CPT1A | 0.034 | 1.00 | NA | NA | 86 | 3.812 | 0.06 | 2.4% | exon 10, F417F | carnitine palmitoyl |
| | | | | | | | | | | | | transferase 1A (liver) |
| dbp.chg | rs3853188 | SCARB2 | 0.038 | 1.00 | NA | NA | 79 | −3.32 | 0.099 | 1.9% | intron 2 | scavenger receptor class B, |
| | | | | | | | | | | | | member 2 |
| dbp.chg | rs6837793 | NPY5R | 0.038 | 1.00 | NA | NA | 83 | −3 | 0.322 | 0.7% | ~9 kb upstream | neuropeptide Y receptor Y5 |
| dbp.chg | PPARA | NA | 0.04 | 1.00 | 0.023 | 4.8% | 91 | −3.64 | 0.073 | 2.2% | | |
| dbp.chg | HL | NA | 0.041 | 1.00 | 0.01 | 6.2% | 80 | −2.15 | 0.039 | 2.9% | | |
| dbp.chg | rs324651 | CHRM2 | 0.045 | 1.00 | 0.018 | 5.2% | 79 | 2.816 | 0.042 | 2.9% | ~400 bp | cholinergic receptor, |
| | | | | | | | | | | | upstream | muscarinic 2 |

TABLE 14
SNPs with statistical significance level of 0.05 for change in body mass (bms)

| var | snp | gene | pval | adj | mpv | mr² | dgef | coeff | apv | ar² | SNP type | Gene Name |
|---|---|---|---|---|---|---|---|---|---|---|---|---|
| bms.chg | Total | 0 | 0.282 | 0.28 | 2E−11 | 72.0% | 54 | −0.24 | 6E−10 | 53.9% | | |
| bms.chg | rs1801278 | IRS1 | 7E−04 | 0.19 | 5E−07 | 16.8% | 90 | −2.96 | 1E−05 | 9.8% | exon 1, R97 1 G | insulin receptor |
| | | | | | | | | | | | | substrate 1 |
| bms.chg | rs375607 | GABRA2 | 0.002 | 0.48 | 0.003 | 2.5% | 95 | 3.022 | 2E−04 | 7.0% | 5′ UTR, (map | (gamma-aminobutyric |
| | | | | | | | | | | | shows intron 1) | acid (GABA) A receptor |
| | | | | | | | | | | | | alpha 2 |
| bms.chg | rs2070424 | SOD1 | 0.007 | 0.88 | 0.028 | 2.6% | 94 | −2.77 | 0.002 | 4.9% | intron 3 | superoxide dismnutase 1, |
| | | | | | | | | | | | | soluble (amyotrophic |
| | | | | | | | | | | | | lateral sclerosis 1 (adult)) |
| bms.chg | rs676643 | HTR1D | 0.013 | 0.98 | NA | NA | 96 | −4.41 | 0.003 | 4.4% | ~200 bp upstream | 5-hydroxytryptamine |
| | | | | | | | | | | | | (serotonin) receptor ID |
| bms.chg | rs870995 | PIK3CA | 0.018 | 0.99 | NA | NA | 92 | −1.04 | 0.038 | 2.1% | ~3.3 kb upstream | phosphoinositide-3- |
| | | | | | | | | | | | | kinase, catalytic, |
| | | | | | | | | | | | | alpha polypeptide |
| bms.chg | rs2807071 | OAT | 0.02 | 1.00 | NA | NA | 92 | −1.37 | 0.069 | 1.6% | inton 3 | ornithine aminotranferase |
| | | | | | | | | | | | | (gyrate atrophy) |
| bms.chg | rs10508244 | PFKP | 0.022 | 1.00 | NA | NA | 92 | 1.701 | 0.023 | 2.5% | intron 10 | phosphofructokinase, |
| | | | | | | | | | | | | platelet |
| bms.chg | rs2162189 | SST | 0.022 | 1.00 | 3E−04 | 7.8% | 96 | 1.964 | 0.022 | 2.5% | ~2.5 kbp | somatostatin |
| | | | | | | | | | | | upstream | |
| bms.chg | rs4792887 | CRHR1 | 0.028 | 1.00 | 0.007 | 4.1% | 97 | −1.51 | 0.009 | 3.3% | intron 1 | corticotropin releasing |
| | | | | | | | | | | | | hormone receptor 1 |
| bms.chg | HL | NA | 0.03 | 1.00 | NA | NA | 86 | −1.19 | 0.065 | 1.6% | | |
| bms.chg | LPL | NA | 0.031 | 1.00 | 0.003 | 5.0% | 80 | −1.57 | 0.042 | 2.0% | | |
| bms.chg | rs2296189 | FLT1 | 0.036 | 1.00 | NA | NA | 97 | 1.354 | 0.054 | 1.8% | exon 24 P1068P | fms-related tyrosine |
| | | | | | | | | | | | | kinase 1 (vascular |
| | | | | | | | | | | | | endothelial growth |
| | | | | | | | | | | | | factor/vascular |
| | | | | | | | | | | | | permeability factor |
| | | | | | | | | | | | | receptor |
| bms.chg | rs6700734 | TNFSF6 | 0.038 | 1.00 | 2E−05 | 11.3% | 92 | −1.06 | 0.099 | 1.3% | intron 2 | tumor necrosis factor |
| | | | | | | | | | | | | (ligand) superfamily, |
| | | | | | | | | | | | | member 6 |
| bms.chg | rs1255 | MDH1 | 0.04 | 1.00 | IE−04 | 9.0% | 95 | 1.053 | 9E−04 | 5.5% | intron 4 | malate dehydrogenase |
| | | | | | | | | | | | | 1, NAD (soluble) |
| bms.chg | rs1440451 | HTR5A | 0.042 | 1.00 | 0.002 | 5.2% | 92 | 2.116 | 0.051 | 1.8% | intron 1 | 5-hydroxytryptamine |
| | | | | | | | | | | | | (serotonin) receptor 5A |
| bms.chg | rs3769671 | POMC | 0.047 | 1.00 | NA | NA | 88 | 2.027 | 0.248 | 0/6% | intron 1 | proopimelanocortin |
| | | | | | | | | | | | | (adrenocotropin/beta- |
| | | | | | | | | | | | | lipotropin/alpha- |
| | | | | | | | | | | | | melanocyte |
| | | | | | | | | | | | | stimulating hormone/ |
| | | | | | | | | | | | | beta-melanocyte |
| | | | | | | | | | | | | stimulating hormone/ |
| | | | | | | | | | | | | beta-entrophin) |
| bms.chg | rs722341 | ABCC8 | 0.048 | 1.00 | 0.009 | 3.8% | 94 | 1.336 | 0.444 | 0.3% | intron 7 | ATP-binding cassette, |
| | | | | | | | | | | | | sub-family C |
| | | | | | | | | | | | | (CFTR/MRP), member 8 |
| bms.chg | rs1041163 | VCAM1 | 0.049 | 1.00 | 0.008 | 3.9% | 90 | 1.134 | 0.208 | 0.7% | ~150 bp upstream | vascular cell |
| | | | | | | | | | | | | adhesion molecule 1 |
| bms.chg | rs2742115 | OLR1 | 0.05 | 1.00 | NA | NA | 90 | 1.012 | 0.473 | 0.2% | intron 1 | oxidised low density |
| | | | | | | | | | | | | lipoprotein (lectin-like) |
| | | | | | | | | | | | | receptor 1 |

TABLE 15
SNPs with statistical significance level of 0.05 for change in body mass index (bmi)

| var | snp | gene | pval | adj | mpv | mr2 | degf | coeff | apv | ar2 | SNP type | Gene Name |
|---|---|---|---|---|---|---|---|---|---|---|---|---|
| bmi.chg | Total | 0 | 0.245 | 0.25 | 3E−06 | 50.2% | 55 | 0.102 | 4E−09 | 47.6% | | |
| bmi.chg | rs1801278 | IRS1 | 7E−04 | 0.17 | 2E−04 | 14.7% | 90 | −0.99 | 2E−05 | 10.1% | exon 1, R971G | insulin receptor |
| | | | | | | | | | | | | substrate 1 |
| bmi.chg | rs3756007 | GABRA2 | 0.002 | 0.37 | NA | NA | 95 | 1.036 | 2E−04 | 7.6% | 5′ UTR, (map | gamma-aminobutyric |
| | | | | | | | | | | | shows intron 1 | acid (GABA) A receptor, |
| | | | | | | | | | | | | alpha 2 |
| bmi.chg | rs676643 | HTR1D | 0.007 | 0.88 | NA | NA | 96 | −0.5 | 0.009 | 3.7% | ~200 bp upstream | 5-hydroxytryptamine |
| | | | | | | | | | | | | (serotonin) receptor 1D |
| bmi.chg | rs2070424 | SOD1 | 0.008 | 0.88 | 0.126 | 2.2% | 94 | −0.92 | 5E−04 | 6.6% | intron 3 | superoxide dismutase |
| | | | | | | | | | | | | 1, soluble (amyotrophic |
| | | | | | | | | | | | | lateral selerosis 1 (adult) |
| bmi.chg | rs870995 | PIK3CA | 0.011 | 0.96 | NA | NA | 92 | −0.37 | 0.029 | 2.5% | ~3.3 kb upstream | phosphoinositide-3- |
| | | | | | | | | | | | | kinase, catalytic, alpha |
| | | | | | | | | | | | | polypeptide |
| bmi.chg | rs2807071 | OAT | 0.021 | 1.00 | NA | NA | 92 | −0.45 | 0.093 | 1.5% | intron 3 | ornithine |
| | | | | | | | | | | | | aminotransferase |
| | | | | | | | | | | | | (gyrate atrophy) |
| bmi.chg | rs2162189 | SST | 0.021 | 1.00 | 0.006 | 7.4% | 96 | 0.658 | 0.059 | 1.9% | ~2.5 kbp | somatostatin |
| | | | | | | | | | | | upstream | |
| bmi.chg | rs1440451 | HTR5A | 0.024 | 1.00 | 0.025 | 4.8% | 92 | 0.772 | 0.048 | 2.0% | intron 1 | 5-hydroxytryptamine |
| | | | | | | | | | | | | (serotonin) receptor 5A |
| bmi.chg | LPL | NA | 0.024 | 1.00 | 0.03 | 4.5% | 80 | −0.54 | 0.017 | 3.0% | | |
| bmi.chg | rs10508244 | PFKP | 0.027 | 1.00 | NA | NA | 92 | 0.55 | 0.059 | 1.9% | intron 10 | phosphofructokinase, |
| | | | | | | | | | | | | platelet |
| bmi.chg | rs4792887 | CRHR1 | 0.037 | 1.00 | 0.025 | 4.8% | 97 | −0.48 | 0.007 | 3.9% | intron 1 | corticotropin releasing |
| | | | | | | | | | | | | hormone receptor 1 |
| bmi.chg | rs3769671 | POMC | 0.04 | 1.00 | NA | NA | 88 | 0.705 | 0.213 | 0.8% | intron 1 | proopiomelanocortin |
| | | | | | | | | | | | | (adrenocorticotropin/ |
| | | | | | | | | | | | | beta-lipotropin/alpha- |
| | | | | | | | | | | | | melanocyte stimulating |
| | | | | | | | | | | | | hormone/beta- |
| | | | | | | | | | | | | melanocyte stimulating |
| | | | | | | | | | | | | hormone/beta- |
| | | | | | | | | | | | | endorphin) |
| bmi.chg | rs167771 | DRD3 | 0.04 | 1.00 | 0.206 | 1.5% | 90 | 0.398 | 0.39 | 0.4% | intron 3 | dopamine receptor D3 |
| bmi.chg | rs936960 | LIPC | 0.045 | 1.00 | 0.001 | 10.3% | 92 | 0.494 | 0.853 | 0.0% | intron 1 | lipase, hepatic |
| bmi.chg | rs2296189 | FLT1 | 0.046 | 1.00 | NA | NA | 97 | 0.427 | 0.055 | 1.9% | exon 24, P1068P | fms-related tyrosine |
| | | | | | | | | | | | | kinase 1 (vascular |
| | | | | | | | | | | | | endothelial growth |
| | | | | | | | | | | | | factor/vascular |
| | | | | | | | | | | | | permeability |
| | | | | | | | | | | | | factor receptor) |

TABLE 16
SNPs with statistical significance level of 0.05 for change in waist size.

| var | snp | gene | pval | adj | mpv | mr2 | degf | coeff | apv | ar2 | SNP type | Gene Name |
|---|---|---|---|---|---|---|---|---|---|---|---|---|
| waist.chg | Total | 0 | 0.874 | 0.87 | 3E−04 | 22.8% | 82 | 0.424 | 0.003 | 19.0% | | |
| waist.chg | rs6700734 | TNFSF6 | 0.01 | 0.94 | 0.013 | 6.1% | 91 | −0.65 | 0.006 | 5.9% | intron 2 | tumor necrosis factor (ligand) |
| | | | | | | | | | | | | superfamily, member 6 |
| waist.chg | rs2269935 | PFKM | 0.013 | 0.97 | 0.034 | 4.4% | 95 | −0.68 | 0.016 | 4.5% | ~700 bp | phosphofructokinase, muscle |
| | | | | | | | | | | | upstream | |
| waist.chg | rs4933200 | ANKRD1 | 0.019 | 1.00 | 0.015 | 5.8% | 93 | −0.72 | 0.074 | 2.4% | intron 5 | ankyrin repeat domain 1 |
| | | | | | | | | | | | | (cardiac muscle) |
| waist.chg | rs10082776 | RARG | 0.024 | 1.00 | NA | NA | 93 | −0.84 | 0.146 | 1.6% | intron 2 | retinoic acid receptor, gamma |
| | | | | | | | | | | | (untranslated) | |
| waist.chg | rs1935349 | HTR7 | 0.033 | 1.00 | NA | NA | 95 | −0.65 | 0.484 | 0.4% | intron 1 (MT) | 5-hydroxytryptamine |
| | | | | | | | | | | | | (serotonin) receptor 7 |
| | | | | | | | | | | | | (adenylate cyclase-coupled) |
| waist.chg | rs2514869 | ANGPT1 | 0.036 | 1.00 | 0.01 | 6.5% | 90 | 0.628 | 0.088 | 2.2% | intron 8 | angiopoietin 1 |
| waist.chg | LPL | NA | 0.039 | 1.00 | NA | NA | 79 | −0.69 | 0.141 | 1.6% | | |
| waist.chg | rs2020933 | SLC6A4 | 0.044 | 1.00 | NA | NA | 94 | −0.83 | 0.454 | 0.4% | intron 1 | solute carrier family 6 |
| | | | | | | | | | | | | (neurotransmitter transporter, |
| | | | | | | | | | | | | serotonin), member 4 |

TABLE 17
SNPs with statistical significance level of 0.05 for change in percent fat (pcfat)

| var | snp | gene | pval | adj | mpv | mr2 | degf | coeff | apv | ar2 | SNP type | Gene Name |
|---|---|---|---|---|---|---|---|---|---|---|---|---|
| pcfat.chg | Total | 0 | 0.73 | 0.73 | 8E−06 | 33.6% | 80 | 0.37 | 2E−06 | 29.2% | | |
| pcfat.chg | rs600728 | TEK | 0.002 | 0.36 | 6E−04 | 10.5% | 92 | −1.91 | 4E−04 | 8.7% | intron 1 | TEK tyrosine kinase, |
| | | | | | | | | | | | | endothelial (venous |
| | | | | | | | | | | | | malformations, multiple |
| | | | | | | | | | | | | cutaneous and mucosal) |
| pcfat.chg | rs8178990 | CHAT | 0.014 | 0.98 | 0.03 | 4.1% | 95 | −1.5 | 0.006 | 5.1% | exon 4, F125L (MT) | choline acetyltransferase |
| pcfat.chg | rs1290443 | RARB | 0.02 | 1.00 | 0.013 | 5.4% | 85 | 0.846 | 0.02 | 3.6% | intron 3 (MT) | retinoic acid receptor, beta |
| pcfat.chg | rs722341 | ABCC8 | 0.038 | 1.00 | 0.04 | 3.6% | 93 | 0.934 | 0.015 | 3.9% | intron 7 | ATP-binding cassette, |
| | | | | | | | | | | | | sub-family C (CFTR/MRP), |
| | | | | | | | | | | | | member 8 |
| pcfat.chg | rs885834 | CHAT | 0.046 | 1.00 | 0.033 | 3.9% | 93 | −0.6 | 0.028 | 3.2% | ~450 bp upstream | choline acetyltransferase |
| pcfat.chg | rs2162189 | SST | 0.046 | 1.00 | NA | NA | 95 | 1.141 | 0.123 | 1.6% | ~2.5 kbp upstream | somatostatin |
| pcfat.chg | rs2070424 | SOD1 | 0.05 | 1.00 | 0.008 | 6.1% | 93 | −1.38 | 0.029 | 3.2% | intron 3 | superoxide dismutase 1, |
| | | | | | | | | | | | | soluble (amyotrophic |
| | | | | | | | | | | | | lateral sclerosis 1 (adult)) |

TABLE 18
SNPs with statistical significance level of 0.05 for change in weight normalized maximum oxygen uptake (vmax)

| var | snp | gene | pval | adj | mpv | mr2 | degf | coeff | apv | ar2 | SNP type | Gene Name |
|---|---|---|---|---|---|---|---|---|---|---|---|---|
| vmax.chg | Total | 0 | 0.092 | 0.09 | 1E−06 | 44.8% | 72 | −0.08 | 7E−10 | 52.8% | | |
| vmax.chg | rs4149056 | SLCO1B1 | 7E−04 | 0.19 | 8E−04 | 9.4% | 93 | 1.917 | 7E−06 | 10.6% | exon 5, A174V | solute carrier |
| | | | | | | | | | | | | organic anion |
| | | | | | | | | | | | | transporter family, |
| | | | | | | | | | | | | member 1B1 |
| vmax.chg | rs2298122 | DRD1IP | 0.002 | 0.43 | NA | NA | 92 | −1.69 | 3E−04 | 6.6% | intron 1 | dopamine receptor D1 |
| | | | | | | | | | | | | interacting protein |
| vmax.chg | rs563895 | AVEN | 0.003 | 0.60 | 0.039 | 3.4% | 97 | −1.9 | 4E−04 | 6.4% | intron 2 | apoptosis, caspase |
| | | | | | | | | | | | | activation inhibitor |
| vmax.chg | rs7412 | APOE | 0.009 | 0.93 | 0.04 | 3.4% | 96 | 1.732 | 0.022 | 2.6% | exon 3, C176R | apolipoprotein B |
| vmax.chg | rs2702285 | AVEN | 0.013 | 0.97 | NA | NA | 93 | −1.19 | 0.756 | 0.0% | intron 1 (MT) | apoptosis, caspase |
| | | | | | | | | | | | | activation inhibitor |
| vmax.chg | rs5896 | F2 | 0.015 | 0.98 | 0.005 | 6.4% | 91 | −1.53 | 0.005 | 3.8% | exon 6, M165T | coagulation factor |
| | | | | | | | | | | | | II (thrombin) |
| vmax.chg | rs1356413 | PIK3CA | 0.015 | 0.99 | 0.008 | 5.8% | 92 | −2.22 | 0.001 | 5.4% | intron 16 | phosphoinositide-3- |
| | | | | | | | | | | | | kinase, cetalytic, |
| | | | | | | | | | | | | alpha polypeptide |
| vmax.chg | rs3917550 | PON1 | 0.016 | 0.99 | 0.002 | 7.7% | 95 | −1.41 | 0.01 | 3.3% | intron 7 | paraoxonase 1 |
| vmax.chg | rs662 | PON1 | 0.017 | 0.99 | NA | NA | 94 | −1.2 | 0.636 | 0.1% | | paraoxonase 1 |
| vmax.chg | rs10460960 | CCK | 0.023 | 1.00 | NA | NA | 95 | 1.441 | 0.041 | 2.0% | ~2.5 kb upstream | cholecystokinin |
| vmax.chg | rs7520974 | CHRM3 | 0.025 | 1.00 | NA | NA | 93 | −1 | 0.048 | 1.9% | ~4 kb upstrearn | cholinergic receptor, |
| | | | | | | | | | | | | muscarinic 3 |
| vmax.chg | rs1396862 | CRHR1 | 0.03 | 1.00 | NA | NA | 96 | 1.275 | 0.05 | 1.9% | intron 4 | corticotropin releasing |
| | | | | | | | | | | | | hormone receptor 1 |
| vmax.chg | rs1801714 | ICAM1 | 0.036 | 1.00 | 0.681 | 0.1% | 88 | 0.919 | 0.731 | 0.1% | exon 5, P352L | intercellular adhesion |
| | | | | | | | | | | | | molecule 1 (CD54), |
| | | | | | | | | | | | | human rhinovirus |
| | | | | | | | | | | | | receptor |
| vmax.chg | rs8178990 | CHAT | 0.04 | 1.00 | NA | NA | 96 | 1.923 | 0.123 | 1.1% | exon 4, F125L | choline |
| | | | | | | | | | | | (MT) | acetyltransferase |
| vmx.chg | rs1800871 | IL10 | 0.042 | 1.00 | 0.01 | 5.4% | 96 | 1.163 | 0.043 | 2.0% | ~700 bp upstream | interleukin 10 |
| vmax.chg | rs334555 | GSK3B | 0.043 | 1.00 | NA | NA | 93 | 1.151 | 0.045 | 1.9% | intron 1 | glycogen synthase |
| | | | | | | | | | | | | kinase 3 beta |
| vmax.chg | rs2296189 | FLT1 | 0.044 | 1.00 | 0.049 | 3.1% | 97 | −1.29 | 0.012 | 3.1% | exon 24, P1068P | fms-related tyrosine |
| | | | | | | | | | | | | kinase 1 (vascular |
| | | | | | | | | | | | | endothelial growth |
| | | | | | | | | | | | | factor/vascular |
| | | | | | | | | | | | | permeability |
| | | | | | | | | | | | | factor receptor) |
| vmax.chg | rs6809631 | PPARG | 0.046 | 1.00 | NA | NA | 85 | −1.06 | 0.886 | 0.0% | intron 1 | peroxisome |
| | | | | | | | | | | | | proliferative |
| | | | | | | | | | | | | activated receptor, |
| | | | | | | | | | | | | gamma |

TABLE 19
SNPs with statistical significance level of 0.05 for change in maximum oxygen uptake (vmaxl)

| var | snp | gene | pval | adj | mpv | mr2 | degf | coeff | apv | ar2 | SNP type | Gene Name |
|---|---|---|---|---|---|---|---|---|---|---|---|---|
| vmaxl.chg | Total | 0 | 0.552 | 0.55 | 3E−08 | 50.6% | 74 | −0.12 | 8E−10 | 45.8% | | |
| vmaxl.chg | rs5896 | F2 | 0.006 | 0.79 | 5E−05 | 12.2% | 91 | −0.13 | 3E−04 | 7.1% | exon 6, M165T | coagulation factor II |
| | | | | | | | | | | | | (thrombin) |
| vmaxl.chg | rs334555 | GSK3B | 0.006 | 0.81 | 1E−04 | 11.3% | 93 | 0.12 | 9E−05 | 8.5% | intron 1 | glycogen synthase kinase |
| | | | | | | | | | | | | 3 beta |
| vmaxl.chg | rs4149056 | SLCO1B1 | 0.007 | 0.88 | 0.012 | 4.5% | 93 | 0.119 | 0.001 | 5.7% | exon 5, A174V | solute carrier organic |
| | | | | | | | | | | | | anion transporter family, |
| | | | | | | | | | | | | member 1B1 |
| vmaxl.chg | rs563895 | AVEN | 0.009 | 0.93 | 0.055 | 2.5% | 97 | −0.13 | 0.017 | 3.0% | intron 2 | apoptosis, caspase |
| | | | | | | | | | | | | activation inhibitor |
| vmaxl.chg | rs4072032 | PECAM1 | 0.013 | 0.97 | NA | NA | 86 | −0.1 | 0.05 | 2.0% | intron 1 | platelet/endothelial cell |
| | | | | | | | | | | | | adhesion molecule (CD31 |
| | | | | | | | | | | | | antigen) |
| vmaxl.chg | rs722341 | ABCC8 | 0.017 | 0.99 | 0.005 | 5.5% | 94 | 0.126 | 0.03 | 2.5% | intron 7 | ATP-binding cassette, sub- |
| | | | | | | | | | | | | family C (CFTR/MRP), |
| | | | | | | | | | | | | member 8 |
| vmaxl.chg | APOE4 | NA | 0.022 | 1.00 | 0.022 | 3.7% | 117 | 0.105 | 0.005 | 4.2% | | |
| vmaxl.chg | rs2515449 | MCPH1 | 0.026 | 1.00 | 0.006 | 5.4% | 91 | 0.143 | 0.045 | 2.1% | intron 9 | microcephaly, primary |
| | | | | | | | | | | | | autosomal recessive 1 |
| vmaxl.chg | rs1805002 | CCKBR | 0.032 | 1.00 | 0.101 | 1.8% | 96 | 0.172 | 0.016 | 3.1% | I125V, exon 2 | cholecystokinin B receptor |
| vmaxl.chg | rs2298122 | DRD1IP | 0.044 | 1.00 | NA | NA | 92 | −0.09 | 0.015 | 3.1% | intron 1 | dopamine receptor D1 |
| | | | | | | | | | | | | interacting protein |
| vmaxl.chg | rs7412 | APOE | 0.045 | 1.00 | 0.268 | 0.8% | 96 | 0.105 | 0.169 | 1.0% | exon 3, C176R | apolipoprotein E |
| vmaxl.chg | rs1396862 | CRHR1 | 0.049 | 1.00 | 0.046 | 2.8% | 96 | 0.091 | 0.009 | 3.6% | intron 4 | corticotropin releasing |
| | | | | | | | | | | | | hormone receptor 1 |

TABLE 20
Covariates

| fac | lev | N | Gt | Idl | n | hdl | n | logtg | n | glu | n | Idlsm | n | hdllg | n | sbp |
|---|---|---|---|---|---|---|---|---|---|---|---|---|---|---|---|---|
| All | all | 120 | 100 | 1.79 | 119 | 0.41 | 119 | −0.12 | 120 | −0.16 | 106 | 0.42 | 106 | −0.74 | 105 | −2.05 |
| site | Florida | 15 | 15 | 0.27 | 15 | 0.73 | 15 | −0.15 | 15 | −3.00 | 15 | 1.10 | 1.1 | 3.86 | 11 | −4.53 |
| site | HartHosp | 11 | 8 | −4.68 | 10 | 0.70 | 10 | −0.14 | 11 | −1.33 | 6 | 2.38 | 9 | −4.56 | 9 | −1.64 |
| site | Michigan | 23 | 17 | 2.97 | 23 | 2.22 | 23 | −0.11 | 23 | 0.05 | 22 | 5.80 | 22 | 0.62 | 22 | −4.35 |
| site | Mississippi | 22 | 19 | −2.68 | 22 | 1.11 | 22 | −0.06 | 22 | −0.14 | 22 | 1.66 | 19 | −0.62 | 19 | −3.77 |
| site | NewBritian | 2 | 2 | 9.40 | 2 | −3.50 | 2 | −0.26 | 2 | 7.00 | 1 | 0.00 | 1 | −14.00 | 1 | 17.00 |
| Site | UConn | 9 | 7 | 2.54 | 9 | −1.94 | 9 | −0.08 | 9 | −5.57 | 7 | −18.36 | 9 | −1.83 | 9 | 6.67 |
| site | UMass | 20 | 18 | 11.31 | 20 | 1.48 | 20 | −0.12 | 20 | 2.38 | 16 | −6.48 | 20 | −0.03 | 20 | −2.95 |
| site | WVU | 18 | 14 | −1.17 | 18 | −2.78 | 18 | −0.16 | 18 | 1.88 | 17 | 9.75 | 15 | −3.61 | 14 | −0.67 |
| gender | female | 63 | 54 | 1.57 | 62 | −0.48 | 62 | −0.09 | 63 | 0.31 | 58 | 2.78 | 55 | −2.30 | 55 | −2.22 |
| gender | male | 57 | 46 | 2.03 | 57 | 1.37 | 57 | −0.14 | 57 | −0.73 | 48 | −2.13 | 51 | 0.96 | 50 | −1.86 |
| heritage | AfricanAm | 2 | 2 | −3.80 | 2 | 1.75 | 2 | 0.00 | 2 | 5.50 | 2 | −39.15 | 2 | 8.45 | 2 | 8.00 |
| heritage | Asian | 2 | 2 | −13.75 | 2 | −3.00 | 2 | −0.02 | 2 | 6.50 | 2 | 0.00 | 1 | 5.20 | 1 | 1.00 |
| heritage | Caucasian | 111 | 92 | 2.26 | 111 | 0.36 | 111 | −0.11 | 111 | −0.40 | 99 | 1.06 | 100 | −1.10 | 99 | −2.29 |
| heritage | Hispanic | 5 | 4 | −9.03 | 4 | 2.88 | 4 | −0.30 | 5 | −0.33 | 3 | 5.47 | 3 | 2.93 | 3 | −2.00 |
| alcohol | no | 37 | 33 | −3.65 | 37 | −0.66 | 37 | −0.12 | 37 | 1.33 | 33 | 1.79 | 31 | −1.51 | 31 | 2.49 |
| alcohol | yes | 83 | 67 | 4.25 | 82 | 0.89 | 82 | −0.12 | 83 | −0.84 | 73 | −0.15 | 75 | −0.42 | 74 | −4.07 |
| smoked | no | 82 | 65 | 1.84 | 81 | 0.32 | 81 | −0.12 | 82 | 0.07 | 72 | −0.16 | 70 | −1.80 | 69 | −0.63 |
| smoked | yes | 38 | 35 | 1.68 | 38 | 0.59 | 38 | −0.11 | 38 | −0.65 | 34 | 1.54 | 36 | 1.28 | 36 | −5.11 |
| meds | no | 77 | 64 | 1.09 | 77 | 1.03 | 77 | −0.13 | 77 | 0.52 | 66 | −0.45 | 67 | 0.86 | 66 | −2.31 |
| meds | yes | 43 | 36 | 3.08 | 42 | −0.74 | 42 | −0.09 | 43 | −1.28 | 40 | 1.91 | 39 | −3.46 | 39 | −1.58 |

| fac | n | dbp | n | bms | n | bmi | n | waist | n | pcfat | n | vmax | n | vmax1 | n |
|---|---|---|---|---|---|---|---|---|---|---|---|---|---|---|---|
| All | 120 | −2.88 | 120 | −1.18 | 120 | −0.37 | 120 | −0.63 | 118 | −0.93 | 118 | 3.26 | 119 | 0.24 | 119 |
| site | 15 | −3.47 | 15 | −1.58 | 15 | −0.55 | 15 | −0.92 | 15 | 0.11 | 15 | 0.95 | 15 | 0.07 | 15 |
| site | 11 | −0.73 | 11 | −1.44 | 11 | −0.34 | 11 | 0.23 | 10 | −2.13 | 10 | 1.78 | 11 | 0.12 | 11 |
| site | 23 | −4.61 | 23 | −1.07 | 23 | −0.31 | 23 | −1.08 | 23 | 0.34 | 23 | 2.26 | 23 | 0.21 | 23 |
| site | 22 | −2.23 | 22 | −0.25 | 22 | −0.11 | 22 | −0.52 | 22 | −0.66 | 22 | 3.43 | 22 | 0.27 | 22 |
| site | 2 | 9.00 | 2 | −5.45 | 2 | −1.94 | 2 | −2.63 | 2 | −3.13 | 2 | 1.05 | 2 | −0.02 | 2 |
| Site | 9 | −2.22 | 9 | −1.62 | 9 | −0.54 | 9 | −0.08 | 9 | −2.46 | 9 | 1.73 | 9 | 0.08 | 9 |
| site | 20 | −4.55 | 20 | −0.69 | 20 | −0.17 | 20 | −0.53 | 19 | −2.65 | 19 | 2.60 | 19 | 0.17 | 19 |
| site | 18 | −2.11 | 18 | −1.83 | 18 | −0.59 | 18 | −0.55 | 18 | −0.28 | 18 | 8.86 | 18 | 0.63 | 18 |
| gender | 63 | −2.90 | 63 | −0.67 | 63 | −0.23 | 63 | −0.27 | 62 | −1.35 | 62 | 2.35 | 63 | 0.15 | 63 |
| gender | 57 | −2.86 | 57 | −1.75 | 57 | −0.53 | 57 | −1.02 | 56 | −0.47 | 56 | 4.28 | 56 | 0.34 | 56 |
| heritage | 2 | −1.50 | 2 | −0.40 | 2 | −0.17 | 2 | −0.75 | 2 | 0.02 | 2 | 0.75 | 2 | 0.12 | 2 |
| heritage | 2 | −2.00 | 2 | −2.51 | 2 | −1.00 | 2 | −1.38 | 2 | −1.26 | 2 | 2.85 | 2 | 0.08 | 2 |
| heritage | 111 | −3.05 | 111 | −1.28 | 111 | −0.40 | 111 | −0.66 | 109 | −0.88 | 109 | 3.37 | 110 | 0.25 | 110 |
| heritage | 5 | 0.00 | 5 | 1.08 | 5 | 0.47 | 5 | 0.55 | 5 | −2.41 | 5 | 1.93 | 50 | 0.17 | 5 |
| alcohol | 37 | −1.84 | 37 | −0.64 | 37 | −0.18 | 37 | −0.40 | 36 | −1.05 | 36 | 3.83 | 37 | 0.32 | 37 |
| alcohol | 83 | −3.35 | 83 | −1.42 | 83 | −0.45 | 83 | −0.73 | 82 | −0.88 | 82 | 3.00 | 82 | 0.20 | 82 |
| smoked | 82 | −2.41 | 82 | −1.41 | 82 | −0.42 | 82 | −0.63 | 80 | −0.95 | 80 | 2.94 | 82 | 0.19 | 82 |
| smoked | 38 | −3.89 | 38 | −0.69 | 38 | −0.25 | 38 | −0.62 | 38 | −0.84 | 38 | 3.96 | 37 | 0.35 | 37 |
| meds | 77 | −2.36 | 77 | −1.54 | 77 | −0.46 | 77 | −0.80 | 76 | −0.81 | 76 | 3.89 | 76 | 0.27 | 76 |
| meds | 43 | −3.81 | 43 | −0.55 | 43 | −0.21 | 43 | −0.31 | 42 | −1.15 | 42 | 2.15 | 43 | 0.18 | 43 |

TABLE 21
Covariate Model

| Response | Variable | Explains | p |
|---|---|---|---|
| LDL | ldl.pre | 16.3% | 2.50E−06 |
| | age | 4.5% | 0.0103 |
| | hdl.pre | 5.3% | 0.0055 |
| | hdllg.pre | 2.5% | 0.0538 |
| | Total | 28.6% | 2.00E−07 |
| HDL | ldl.pre | 15.6% | 6.60E−07 |
| | hdl.pre | 12.5% | 17.00E−06 |
| | logtg.pre | 5.6% | 0.0021 |
| | hdllg.pre | 5.1% | 0.0031 |
| | vmax.pre | 1.5% | 0.1072 |
| | Total | 40.2% | 8.80E−11 |
| Log(TG) | logtg.pre | 13.4% | 2.10E−05 |
| | dbp.pre | 5.8% | 0.0043 |
| | age | 1.5% | 0.1476 |
| | Total | 20.7% | 5.80E−06 |
| Glu | glu.pre | 35.1% | 1.20E−12 |
| | ldl.pre | 3.0% | 0.0186 |
| | meds | 3.7% | 0.0097 |
| | sbp.pre | 2.3% | 0.0388 |
| | heritage | 3.4% | 0.096 |
| | age | 1.5% | 0.0975 |
| | Total | 49.0% | 1.80E−11 |
| LDL, sm | ldlsm.pre | 20.8% | 6.40E−08 |
| | logtg.pre | 14.5% | 3.90E−06 |
| | ldl.pre | 2.5% | 0.046 |
| | Total | 37.8% | 1.60E−10 |
| HDL, lg | hdllg.pre | 16.7% | 4.40E−06 |
| | bmi.pre | 5.7% | 0.0052 |
| | ldl.pre | 4.6% | 0.0118 |
| | logtg.pre | 4.2% | 0.0169 |
| | glu.pre | 3.0% | 0.0411 |
| | hdl.pre | 1.2% | 0.1957 |
| | Total | 35.4% | 2.80E−07 |
| SBP | sbp.pre | 16.9% | 8.70E−08 |
| | bms.pre | 13.7% | 1.10E−06 |
| | alcohol | 4.7% | 0.0031 |
| | dbp.pre | 4.2% | 0.0053 |
| | meds | 1.8% | 0.0657 |
| | Total | 41.2% | 6.30E−12 |
| DBP | dbp.pre | 22.1% | 1.00E−08 |
| | bms.pre | 8.4% | 0.00021 |
| | vmaxl.pre | 5.1% | 0.00353 |
| | glu.pre | 3.1% | 0.02139 |
| | Total | 38.8% | 8.80E−11 |
| BMS | bms.pre | 12.3% | 8.50E−05 |
| | Total | 12.3% | 8.50E−05 |
| BMI | bms.pre | 11.4% | 0.00016 |
| | Total | 11.4% | 0.00016 |
| Pcfat | pcfat.pre | 12.1% | 4.70E−06 |
| | vmax.pre | 5.3% | 0.0019 |
| | site | 113.2% | 0.0016 |
| | bms.pre | 12.9% | 2.50E−06 |
| | sbp.pre | 1.2% | 0.1312 |
| | Total | 44.7% | 9.20E−10 |
| Vmax | site | 35.5 | 3.80E−10 |
| | logtg.pre | 3.3% | 0.00975 |
| | gender | 7.5% | 0.00012 |
| | vmax.pre | 2.2% | 0.03171 |
| | activity | 0.6% | 0.26945 |
| | Total | 49.2% | 1.20E−11 |
| Vmaxl | site | 27.4% | 2.30E−08 |
| | bms.pre | 5.7% | 0.00059 |
| | logtg.pre | 7.3% | 0.00011 |
| | smoked | 2.1% | 0.03375 |
| | gender | 3.2% | 0.00901 |
| | vmaxl.pre | 6.0% | 0.00042 |
| | alcohol | 0.8% | 0.19498 |
| | Total | 52.5% | 4.80E−12 |

In the SNP screen (step 2), the p-values for each SNP were obtained by adding the SNP to the baseline model and comparing the resulting model improvement with up to 10,000 simulated model improvements using the same data set, but with the genotype data randomly permuted to remove any true association. This method produces a p-value that is a direct, unbiased, and model-free estimate of the probability of finding a model as good as the one tested when the null hypothesis of no association is true. All SNPs with a screening p-value of better than 0.003 were selected to be included in the physiogenomic model (step 3).

Data Analysis. Covariates were analyzed using multiple linear regression and the stepwise procedure. An extended linear model was constructed including the significant covariate and the SNP genotype. SNP genotype was coded quantitatively as a numerical variable indicating the number of minor alleles: 0 for major homozygotes, 1 for heterozygotes, and 2 for minor homozygotes. The F-statistic p-value for the SNP variable was used to evaluate the significance of association. Table 1 lists all SNPs that were tested and their association p-values. The validity of the p-values were tested by performance of an independent calculation of the p-values using permutation testing. To account for the multiple testing of multiple SNPs, adjusted p-values were calculated using Benjamini and Hochbergs false discovery rate (FDR) procedure [Reinere A, Yekutiele D, Benjamini Y: Identifying differentially expressed genes using false discovery rate controlling procedures. Bioinformatics 19:368-375 (2003); Benjamini Y, Hochberg Y: Controlling the false discovery rate: a practical and powerful approach to multiple testing. Journal of the Royal Statistical Society, Series B 57:289-300 (1995); Benjamini Y, Hochberg Y: On the adaptive control of the false discovery rate in multiple testing with independent statistics. Journal of Educational and Behavioral Statistics 25:60-83 (2000).]. In addition, the power for detecting an association based on the Bonferroni multiple comparison adjustment was evaluated. For each SNP, the effect size in standard deviations that was necessary for detection of an association at a power of 80% (20% false negative rate) was calculated using the formula:

$$Δ = \frac{{z}_{α / c} + {z}_{β}}{\sqrt{Nf ( 1 - f )}}$$

where α was the desired false positive rate (α=0.05), β the false negative rate (β=1-Power=0.2), c the number of SNPs, z a standard normal deviate, N the number of subjects, f the carrier proportion, and Δ the difference in change in response between carriers and non-carriers expressed relative to the standard deviation [Rosner B: Fundamentals of Biostatistics. Belmont, Calif.: Wadsworth Publishing Co. (1995).].

LOESS representation. A locally smoothed function of the SNP frequency as it varies with each response was used to visually represent the nature of an association. LOESS (LOcally wEighted Scatter plot Smooth) is a method to smooth data using a locally weighted linear regression [Cleveland, W S: Robust locally weighted regression and smoothing scatterplots. Journal of American Statistical Association 74, 829-836 (1979); Cleveland W S, Devlin S J: Locally Weighted Regression: An Approach to Regression Analysis by Local Fitting. Journal of the American Statistical Association Vol. 83, pp. 596-610 (1988).]. At each point in the LOESS curve, a quadratic polynomial was fitted to the data in the vicinity of that point. The data were weighted such that they contributed less if they were further away, according to the following tricubic function where x was the abscissa of the point to be estimated, the xᵢ were the data points in the vicinity, and d(x) was the maximum distance of x to the xᵢ.

$${w}_{i} = {( 1 - {\vert \frac{x - {x}_{i}}{d ( x )} \vert}^{3} )}^{3}$$

The distribution of change in each parameter in the study population are approximately normal. The potential covariates of age, gender, race, are tested for association with each parameter using multiple linear regression. The LOESS curve will show the localized frequency of the least common allele for sectors of the distribution. For SNPs with a strong association, the marker frequency is significantly different between the high end and the low end of the distribution. Conversely, if a marker is neutral, the frequency is independent of the response and the LOESS curve is essentially flat.

If an allele is more common among patients with high response than among those with low response, the allele is likely to be associated with increased response. Similarly, when the allele is less common in those with high response, the allele is associated with decreased response. Thus, the slope of the curve is an indication of the degree of association.

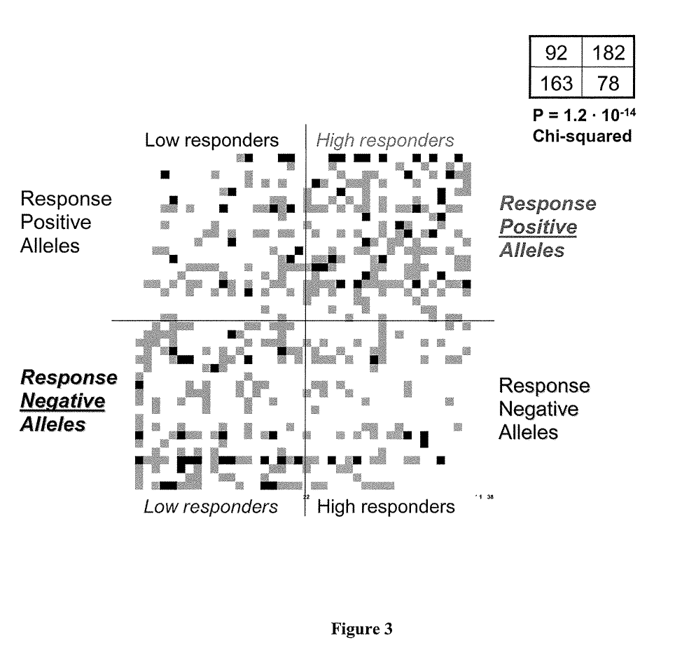

FIG. 3 shows a LOESS fit of the allele frequency as a function of change in body mass (thick line). Individual genotypes (circles) of four SNPs (Serotonin Receptor, Insulin Receptor Substrate, Ornithine Aminotransferase, PI3 Kinase Alpha) are overlaid on the distribution of change in body mass (thin line). Each circle represents a subject, with the horizontal axis specifying the body mass change, and the vertical axis the genotype: bottom—homozygous for major allele, middle—heterozygous, top—homozygous for minor allele.

a. Data analysis. The objective of the statistical analysis is to find a set of physiogenomic factors that together provide a way of predicting the outcome of interest. The association of an individual factor with the outcome may not have sufficient discrimination ability to provide the necessary sensitivity and specificity, but by combining the effect of several such factors the objective is reached. Increased sensitivity and specificity for the cumulative effect on prediction can be achieved through the use of common factors that are statistically independent. The assumptions on which these calculations are based are (a) the factors are independent of each other, (b) the association between each factor and the outcome can be summarized by a modest odds ratio of 1.7, and (c) the prevalence of each physiogenomic factor in the population is 50% and independent of the others. Clearly, the prediction becomes even stronger if the association with the response is stronger or one finds additional predictors. However, factors that are less useful for these types of prediction are those that are less common in the population, or collinear with factors that have already been identified in the prediction model.

### Statistical Plan

a. Data analysis. The objective of the statistical analysis is to find a set of physiogenomic factors that together provide a way of predicting the outcome of interest. The association of an individual factor with the outcome may not have sufficient discrimination ability to provide the necessary sensitivity and specificity, but by combining the effect of several such factors the objective is reached. Increased sensitivity and specificity for the cumulative effect on prediction can be achieved through the use of common factors that are statistically independent. The assumptions on which these calculations are based are (a) the factors are independent of each other, (b) the association between each factor and the outcome can be summarized by a modest odds ratio of 1.7, and (c) the prevalence of each physiogenomic factor in the population is 50% and independent of the others. Clearly, the prediction becomes even stronger if the association with the response is stronger or one finds additional predictors. However, factors that are less useful for these types of prediction are those that are less common in the population, or collinear with factors that have already been identified in the prediction model.

b. Model Building. Discovery of markers affecting response to exercise. A multivariate model was developed for the purpose of predicting a given response (Y) to exercise. A linear model for subjects in a group of patients subjected to exercise was used in which the response of interest can be expressed as follows:

$$Y = {R}_{0} + {\sum }_{i} {α}_{i} {M}_{i} + {\sum }_{j} {β}_{j} {D}_{j} + ɛ$$

where Mᵢ are the dummy marker variables indicating the presence of specified genotypes and Dⱼ are demographic and clinical covariates. The model parameters that are to be estimated from the data are R₀, αᵢ and βⱼ. This model employs standard regression techniques that enable the systematic search for the best predictors. S-plus provides very good support for algorithms that provide these estimates for the initial linear regression models, as well other generalized linear models that may be used when the error distribution is not normal. For continuous variables, generalized additive models, including cubic splines in order to appropriately assess the form for the dose-response relationship may also be considered [Hastie T, Tibshirani R. Generalized additive models. Stat. Sci. 1: 297-318 (1986); Durrleman S, Simon R. Flexible regression models with cubic splines. Statistics in Medicine 8:551-561 (1989).].

In addition to optimizing the parameters, model refinement is performed. The first phase of the regression analysis will consist of considering a set of simplified models by eliminating each variable in turn and re-optimizing the likelihood function. The ratio between the two maximum likelihoods of the original vs. the simplified model then provides a significance measure for the contribution of each variable to the model.

The association between each physiogenomic factor and the outcome is calculated using logistic regression models, controlling for the other factors that have been found to be relevant. The magnitude of these associations are measured with the odds ratio and the corresponding 95% confidence interval, and statistical significance assessed using a likelihood ratio test. Multivariate analyses is used which includes all factors that have been found to be important based on univariate analyses.

Because the number of possible comparisons can become very large in analyses that evaluate the combined effects of two or more genes, the results include a random permutation test for the null hypothesis of no effect for two through five combinations of genes. This is accomplished by randomly assigning the outcome to each individual in the study, which is implied by the null distribution of no genetic effect, and estimating the test statistic that corresponds to the null hypothesis of the gene combination effect. Repeating this process 1000 times will provide an empirical estimate of the distribution for the test statistic, and hence a p-value that takes into account the process that gave rise to the multiple comparisons. In addition, hierarchical regression analysis is considered to generate estimates incorporating prior information about the biological activity of the gene variants. In this type of analysis, multiple genotypes and other risk factors can be considered simultaneously as a set, and estimates will be adjusted based on prior information and the observed covariance, theoretically improving the accuracy and precision of effect estimates [Steenland K, Bray I, Greenland S, Boffetta P. Empirical Bayes adjustments for multiple results in hypothesis-generating or surveillance studies. Ca Epidemiol Biomarkers Prev. 9:895-903 (2000).].

c. Power calculations. The power available for detecting an odds ratio (OR) of a specified size for a particular allele was determined on the basis of a significance test on the corresponding difference in proportions using a 5% level of significance. The approach for calculating power involved the adaptation of the method given by Rosner [Rosner B: Fundamentals of Biostatistics. Belmont, Calif.: Wadsworth Publishing Co. (1995).]. The SNPs that are explored in this research are not so common as to have prevalence of more than 35%, but rather in the range of 10-15%. Therefore, it is apparent that the study has at least 80% power to detect odds ratios in the range of 1.6-1.8, which are modest effects.

d. Model validation. A cross-validation approach is used to evaluate the performance of models by separating the data used for parameterization (training set) from the data used for testing (test set). The approach randomly divides the population into the training set, which will comprise 80% of the subjects, and the remaining 20% will be the test set. The algorithmic approach is used for finding a model that can be used for prediction of exercise response that will occur in a subject using the data in the training set. This prediction equation is then used to prepare an ROC curve that provides an independent estimate of the relationship between sensitivity and specificity for the prediction model.

e. Patient Physiotype. Tables 22 through 34 show a collection of physiotypes for the outcomes log of blood triglyceride level (logTG); blood LDL cholesterol level (LDL); blood HDL cholesterol level (HDL); LDL cholesterol, small fraction level (LDLSM); HDL cholesterol, large fraction level (HDLLG); blood glucose level (GLU); systolic blood pressure (SBP); diastolic blood pressure (DSP); body mass (BMS); body mass index (BMI); fat percentage (PFAT); weight normalized maximum oxygen uptake (VMAX); maximum oxygen uptake (VMAXL). Each physiotype in this particular embodiment consists of a selection of markers, and intercept value (C), and a coefficient (cᵢ) for each marker. For example, the LDL physiotype, in one embodiment, consists of the markers rs2005590, rs1041163, rs1800471, rs1799978, rs870995, rs707922, rs1398176, and rs5092, and the corresponding coefficients −0.53177, −0.29832, −0.69604, 0.92244, 0.28492, −0.25665, 0.26321, and 0.26693, respectively. The predicted LDL response for a given individual is then given by the formula:

$$Δ LDL = C + {\sum }_{i} {c}_{i} {g}_{i}$$

where C is the intercept, the cᵢ are the coefficients and the gᵢ are the genotypes, coded 0 for the wild type allele homozygote, 1 for the heterozygote, and 2 for the variant allele homozygote.

In this embodiment, the physiotype consists of a linear regression model with no interactions. In another embodiment, interaction terms of two or more variables may be added to the model. In other embodiments, the physiotype might consist of a generalized linear regression model, a structural equation model, a Baysian probability network, or any other modeling tool known to the practitioner of the art of statistics.

TABLE 22
LDL Physiotype
LDL

| SNP | Gene | Allele | cᵢ |
|---|---|---|---|
| rs2005590 | APOL4 | TC | −0.53177 |
| rs1041163 | VCAM1 | TC | −0.29832 |
| rs1800471 | TGFB1 | CG | −0.69604 |
| rs1799978 | DRD2 | AG | 0.92244 |
| rs870995 | PIK3CA | AC | 0.28492 |
| rs707922 | APOM | AC | −0.25665 |
| rs1398176 | GABRA4 | TC | 0.26321 |
| rs5092 | APOA4 | AG | 0.26693 |

Intercept (C) = −0.25665

TABLE 23
HDL Physiotype
HDL

| Snp | Gene | Allele | cᵢ |
|---|---|---|---|
| rs1143634 | IL1B | TC | −0.43500 |
| rs5049 | AGT | AG | −0.40011 |
| rs10513055 | PIK3CB | AC | 0.28679 |
| rs1800871 | IL10 | TC | 0.38783 |
| rs3760396 | CCL2 | GC | 0.23682 |
| rs1891311 | HTR7 | AG | −0.42461 |

Intercept (C) = −0.05321

TABLE 24
Triglyceride Physiotype
Log(TG)

| SNP | Gene | Allele | cᵢ |
|---|---|---|---|
| rs908867 | BDNF | AG | 0.36378 |
| rs2240403 | CRHR2 | TC | 0.39108 |
| rs2070586 | DAO | AG | −0.49243 |
| rs10460960 | CCK | AG | −0.31807 |
| rs4121817 | PIK3C3 | AG | 0.35240 |
| rs2276307 | HTR3B | AG | −0.30114 |
| rs11503016 | GABRA2 | TA | −0.35179 |
| rs563895 | AVEN | TC | −0.45039 |
| rs1171276 | LEPR | AG | 0.38428 |
| rs2278718 | MDH1 | AC | 0.19557 |

Intercept (C) = 0.28439

TABLE 25
Blood Glucose Physiotype
Blood Glucose

| SNP | Gene | Allele | cᵢ |
|---|---|---|---|
| rs722341 | ABCC8 | TC | −0.58553 |
| rs3822222 | CCKAR | TC | −0.26087 |
| rs10508244 | PFKP | TC | 0.34507 |
| rs2229126 | ADRA1A | AT | −0.64554 |
| rs1322783 | DISC1 | TC | 0.45206 |
| rs2070424 | SOD1 | AG | 0.59187 |
| rs107540 | CRHR2 | AG | 0.39301 |
| rs1042718 | ADRB2 | AC | 0.27167 |
| rs5361 | SELE | AC | 0.20757 |
| rs322695 | RARB | AG | 0.26464 |

Intercept (C) = −0.60844

TABLE 26
LDL, Small Fraction Physiotype
LDL, small fraction

| SNP | Gene | Allele | cᵢ |
|---|---|---|---|
| rs6131 | SELP | AG | −0.51658 |
| rs1131010 | PECAM1 | TC | 0.61470 |
| rs706713 | PIK3R1 | TC | 0.18704 |
| rs2076672 | APOL5 | TC | −0.23497 |
| rs10890819 | ACAT1 | TC | −0.17035 |
| rs6092 | SERPINE1 | AG | −0.19927 |
| rs4675096 | IRS1 | AG | 0.27763 |
| rs6078 | LIPC | AG | −0.44798 |
| rs659734 | HTR2A | TC | −0.49205 |
| rs885834 | CHAT | AG | −0.11459 |
| rs4917348 | RXRA | AG | 0.12959 |

Intercept (C) = 0.43778

TABLE 27
HDL, Large Fraction Physiotype
HDL, large fraction

| SNP | Gene | Allele | cᵢ |
|---|---|---|---|
| rs5049 | AGT | AG | −0.34284 |
| rs10513055 | PIK3CB | AC | 0.35487 |
| rs1800871 | IL10 | TC | 0.50520 |
| rs3760396 | CCL2 | GC | 0.23609 |
| rs1042718 | ADRB2 | AC | −0.30328 |
| rs4520 | APOC3 | TC | −0.30201 |

Intercept (C) = −0.19238

TABLE 28
Systolic Blood Pressure Physiotype
Systolic Blood Pressure (SBP)

| SNP | Gene | Allele | cᵢ |
|---|---|---|---|
| rs1800871 | IL10 | TC | −0.22252 |
| rs1801105 | HNMT | TC | −0.57128 |
| rs7200210 | SLC12A4 | AG | −0.58447 |
| rs4726107 | PRKAG2 | TC | −0.39913 |
| rs10515070 | PIK3R1 | AT | 0.32686 |
| rs4149056 | SLCO1B1 | TC | 0.29030 |
| rs2298122 | DRD1IP | TG | 0.25008 |
| rs6967107 | WBSCR14 | AC | 0.27530 |

Intercept (C) = −0.04372

TABLE 29
Diastolic Blood Pressure Physiotype
Diastolic Blood Pressure (DBP)

| SNP | Gene | Allele | cᵢ |
|---|---|---|---|
| rs722341 | ABCC8 | TC | 0.49867 |
| rs7556371 | PIK3C2B | AG | 0.31714 |
| rs324651 | CHRM2 | TG | −0.39151 |
| rs4531 | DBH | TG | .34921 |
| rs2067477 | CHRM1 | AC | 0.18135 |

Intercept (C) = −0.06466

TABLE 30
Body Mass Physiotype
Body Mass (BMS)

| SNP | Gene | Allele | cᵢ |
|---|---|---|---|
| rs1041163 | VCAM1 | TC | −0.29515 |
| rs722341 | ABCC8 | TC | −0.34459 |
| rs2070424 | SOD1 | AG | 0.63564 |
| rs1801278 | IRS1 | AG | 0.92951 |
| rs2162189 | SST | AG | −0.77778 |
| rs1255 | MDH1 | AG | −0.53551 |
| rs6700734 | TNFSF6 | AG | 0.44262 |
| rs4792887 | CRHR1 | TC | 0.56361 |
| rs1440451 | HTR5A | CG | −0.78400 |
| rs3756007 | GABRA2 | TC | −0.49891 |

Intercept (C) = 0.072688

TABLE 31
Body Mass Index Physiotype
Body Mass Index (BMI)

| SNP | Gene | Allele | cᵢ |
|---|---|---|---|
| rs2070424 | SOD1 | AG | 0.54996 |
| rs1801278 | IRS1 | AG | 0.96751 |
| rs2162189 | SST | AG | −0.59549 |
| rs4792887 | CRHR1 | TC | 0.54211 |
| rs1440451 | HTR5A | CG | −0.67363 |
| rs936960 | LIPC | AC | −0.74692 |
| rs167771 | DRD3 | AG | −0.20513 |

Intercept (C) = −0.09349

TABLE 32
Percent Fat Physiotype
Percent Fat

| SNP | Gene | Allele | cᵢ |
|---|---|---|---|
| rs722341 | ABCC8 | TC | −0.35036 |
| rs2070424 | SOD1 | AG | 0.54694 |
| rs885834 | CHAT | AG | 0.21700 |
| rs8178990 | CHAT | TC | 0.45493 |
| rs600728 | TEK | AG | 0.57595 |
| rs1290443 | RARB | AG | −0.32370 |

Intercept (C) = −0.13336

TABLE 33
Maximum Oxygen Uptake (Weight Normalized) Physiotype
Vmax

| SNP | Gene | Allele | cᵢ |
|---|---|---|---|
| rs1800871 | IL10 | TC | 0.31429 |
| rs563895 | AVEN | TC | −0.43150 |
| rs4149056 | SLCO1B1 | TC | 0.33662 |
| rs5896 | F2 | TC | −0.11995 |
| rs3917550 | PON1 | TC | −0.31942 |
| rs7412 | APOE | TC | 0.42605 |
| rs2296189 | FLT1 | AG | −0.38902 |
| rs1356413 | PIK3CA | GC | −0.51076 |
| rs1801714 | ICAM1 | TC | 0.04088 |

Intercept (C) = −0.02016

TABLE 34
Maximum Oxygen Uptake Physiotype
Vmaxl

| SNP | Gene | Allele | cᵢ |
|---|---|---|---|
| rs334555 | GSK3B | CG | 0.24614 |
| rs722341 | ABCC8 | TC | 0.25387 |
| rs563895 | AVEN | TC | −0.26463 |
| rs4149056 | SLCO1B1 | TC | 0.24768 |
| rs5896 | F2 | TC | −0.41018 |
| rs7412 | APOE | TC | 0.21231 |
| rs1396862 | CRHR1 | TC | 0.22502 |
| rs2515449 | MCPH1 | AG | 0.38730 |
| rs1805002 | CCKBR | AG | 0.40692 |

Intercept (C) = −0.35478

For each physiolocial parameterm the patient's genotype (0, 1, or 2) is multiplied by the coefficient corresponding to the effect of the particular SNP on a particular response given in the tables above. For each response, the sum

$${\sum }_{i} {c}_{i} {g}_{i}$$

is added to the intercept value C to determine the predicted response to exercise for the patient.

While the SNP ensembles provided in the tables above provide a marked improvement over individual SNPs for predicting the given clinical outcomes, it will be understood that the invention is not limited to these precise ensembles. Rather, each individual SNP and subcombinations of these SNPs are also considered to be within the scope of the invention. Preferably the ensemble is predictive of two or more responses, more preferably, three or more responses, more preferred still, four or more responses. In a preferred embodiment, the ensemble of SNPs is predictive of blood triglyceride level; blood LDL cholesterol level; blood HDL cholesterol level; ratio of total cholesterol to HDL cholesterol; LDL cholesterol, small fraction level; HDL cholesterol, large fraction level; blood glucose level; systolic blood pressure; diastolic blood pressure; body mass; body mass index; waist size, fat percentage; weight normalized maximum oxygen uptake; and maximum oxygen uptake; or any combination thereof.

In the preferred practice of the invention, the ensemble of markers for a particular physiological outcome will comprise at least one SNP having a positive (+) coefficient and at least one SNP having a negative (−) coefficient. In other embodiments, the ensemble will have at least two (or more than two) SNPs, predictive of the same physiological outcome, having a positive (+) coefficient and at least two (or more than two) SNPs, predictive of the same physiological outcome, having a negative (−) coefficient.

The separate physiotypes of Tables 22-34 can be consolidated into a collective physiotype table to provide an ensemble of SNPs predictive of a plurality of physiological responses to exercise. A representative physiotype table showing for one patient is provided in Table 35, wherein the coefficients, cᵢ, have been omitted for brevity and only their relative contribution (+ or −) indicated.

TABLE 35

| | | Genotype | | | | | | | | |
|---|---|---|---|---|---|---|---|---|---|---|
| Marker | | DNA | | Effect of Marker | | | | | | |
| SNP | Gene | Type | Alleles | LDLsm | HDLlrg | TG | Vmax | BMI | BP | Glu |
| rs2033447 | RARB | 0 | TT | + | | | | | | |
| rs1045642 | ABCB1 | 0 | CC | − | | | | | | |
| rs2076672 | APOL5 | 2 | TT | − | | | | | | |
| rs885834 | CHAT | 2 | GG | − | | | | | | |
| rs4917348 | RXRA | 1 | AG | + | | | | | | |
| rs2471857 | DRD2 | 0 | GG | − | | | | | | |
| rs6131 | SELP | 0 | GG | − | | | | | | |
| rs1150226 | HTR3A | 2 | TT | + | | | | | | |
| rs8192708 | PCK1 | 0 | AA | − | | | | | | |
| rs1042718 | ADRB2 | 0 | CC | | − | | | | | |
| rs4520 | APOC3 | 0 | CC | | − | | | | | |
| rs10513055 | PIK3CB | 0 | AA | | + | | | | | |
| rs1800871 | IL10 | 1 | CT | | + | − | | | | |
| rs521674 | ADRA2A | 1 | AT | | − | | | | | |
| rs2070586 | DAO | 0 | GG | | | − | | | | |
| rs7602 | LEPR | 0 | GG | | | + | | | | |
| rs4121817 | PIK3C3 | 0 | GG | | | + | | | | |
| rs11503016 | GABRA2 | 0 | TT | | | − | | | | |
| rs908867 | BDNF | 0 | GG | | | + | | | | |
| rs2278718 | MDH1 | 0 | AA | | | + | | | | |
| rs563895 | AVEN | 0 | CC | | | | − | | | |
| rs3917550 | PON1 | 0 | CC | | | | − | | | |
| rs4149056 | SLCO1B1 | 0 | TT | | | | + | | | |
| rs597316 | CPTIA | 0 | GG | | | | − | | | |
| rs2298122 | DRD1IP | 1 | TG | | | | − | | | |
| rs8178990 | CHAT | 0 | CC | | | | + | | | |
| rs26312 | GHRL | 0 | GG | | | | − | | | |
| rs676643 | HTR1D | 0 | AA | | | | | + | | |
| rs936960 | LIPC | 0 | CC | | | | | − | | |
| rs1801278 | IRS1 | 0 | GG | | | | | + | | |
| rs600728 | TEK | 0 | AA | | | | | + | | |
| rs132642 | APOL3 | 2 | TT | | | | | − | | |
| rs2162189 | SST | 0 | AA | | | | | − | | |
| rs722341 | ABCC8 | 1 | CT | | | | | | + | |
| rs1064344 | CHKB | 0 | GG | | | | | | − | |
| rs662 | PON1 | 0 | AA | | | | | | − | |
| rs3762272 | PKLR | 0 | GG | | | | | | + | |
| rs3822222 | CCKAR | 0 | CC | | | | | | | − |
| rs1398176 | GABRA4 | 0 | CC | | | | | | | − |
| rs322695 | RARB | 0 | GG | | | | | | | + |
| rs1799978 | DRD2 | 0 | AA | | | | | | | − |

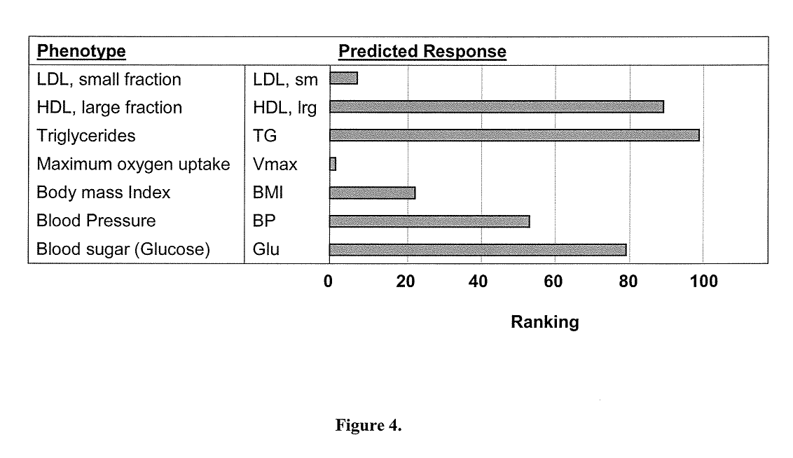

The patient's physiotype may be expressed in a convenient format for the practitioner's assessment of a patient's likely response to exercise, as shown in FIG. 4. The bar chart shown in FIG. 4 shows the patient's rank on a percentile scale of likelihood of response to exercise for the indicated physiological parameters. For example, the particular patient would likely respond favorably to exercise, i.e., better than about 95% of the population, for reduction of triglyceride levels. The physiotype report, such as shown in FIG. 4, predicts and models the individual's innate physiological capacity to respond to exercise. These predictions are independent of baseline status. The ability to isolate the pure genetic contribution to exercise response will be useful to the practitioner, especially in scenarios where baseline data may be difficult to obtain. This type of report enables a patient and physician to evaluate innate physiological capacity and to recommend a wellbeing program incorporating exercise treatment. For example, a given baseline measurement may not be clinically feasible if it is certain to be confounded with drug treatments or diet. In such situations, the physiotype model can be utilized to predict the person's innate physiological capacity to respond, and justify a transition to exercise and judicious use of drugs otherwise prescribed to regulate one or more of the physiological parameters (including, for example, statins, niacin, fibrates, ezitimibe, beta blockers, Ca channel blockers, angiotensinogen receptor blockers, metformin, glitazones, and insulin). This is particularly advantageous in view of the desire of many patients to seek treatment alternatives to medications for control of cardiovascular risk factors. In some cases, for example, the patient may be experiencing drug side effects which are discomforting or disabling or otherwise desire the alternative of preventive healthcare. The possibility of a physiological treatment for such individuals, as opposed to drugs, introduces an entirely new dimension and scientific empowerment to “life style modification”.

The content of all patents, patent applications, published articles, abstracts, books, reference manuals, sequence accession numbers, as cited herein are hereby incorporated by reference in their entireties to more fully describe the state of the art to which the invention pertains.

1-4. (canceled)

5. A method of identifying markers associated with an individual's change in body mass in response to exercise, comprising
assaying genetic material from the individual for the presence or absence of at least one positive marker and at least one torpid marker to produce a physiotype for the individual, wherein the at least one positive marker is a polymorphism in the insulin receptor substrate 1 polynucleotide and the at least one torpid marker is a polymorphism in the gamma-aminobutyric acid (GABA) A receptor, alpha 2 polynucleotide,
wherein the at least one positive marker is associated with a reduction in body mass in response to exercise in the individual and the at least one torpid marker is not associated with a reduction in body mass in response to exercise in the individual.

6. The method of claim 5, wherein the positive marker is rs1801278 and the torpid marker is rs3756007.

7. The method of claim 5, wherein
the at least one positive marker further comprises a marker selected from the group consisting of rs870995, rs600728, rs676643, rs2070424, rs6700734, rs4792887, or a combination of one or more of the foregoing positive markers;
wherein the at least one negative marker further comprises a marker selected from the group consisting of rs6541017, rs1041163, rs722341, rs2162189, rs1255, rs1440451, or a combination of one or more of the foregoing torpid markers.

8. The method of claim 5, wherein
the at least one positive marker further comprises a marker selected from the group consisting of rs600728, rs2070424, rs4792887, or a combination of one or more of the foregoing positive markers;
wherein the at least one negative marker further comprises a marker selected from the group consisting of rs1041163, rs722341, rs2162189, rs1255, rs1440451, or a combination of one or more of the foregoing torpid markers.

9. The method of claim 5, wherein
the at least one positive marker further comprises a marker selected from the group consisting of rs2070424, or a combination of one or more of the foregoing positive markers;
wherein the at least one negative marker further comprises a marker selected from the group consisting of rs2162189, or a combination of one or more of the foregoing torpid markers.

10. The method of claim 5,
further comprising predicting the individual's change in body mass in response to exercise based on the presence or absence of the positive marker and the torpid marker.
